(19) United States
(12) Patent Application Publication
(10) Pub. No.: US 2022/0064696 A1
(43) Pub. Date: Mar. 3, 2022

(54) TARGET ENGAGEMENT ASSAY FOR RAS PROTEINS

(71) Applicant: Promega Corporation, Madison, WI (US)

(72) Inventors: Matthew B. Robers, Madison, WI (US); Joel R. Walker, Madison, WI (US); James Vasta, Madison, WI (US); Cesear Corona, Madison, WI (US)

(73) Assignee: Promega Corporation, Madison, WI (US)

(21) Appl. No.: 17/459,502

(22) Filed: Aug. 27, 2021

Related U.S. Application Data
(60) Provisional application No. 63/160,120, filed on Mar. 12, 2021.
(60) Provisional application No. 63/117,080, filed on Nov. 23, 2020.
(60) Provisional application No. 63/071,694, filed on Aug. 28, 2020.

(51) Int. Cl. C12Q 1/48; C12N 9/10

(52) U.S. Cl. CPC C12Q 1/48; C12N 9/1085

(57) ABSTRACT

Provided herein are systems, methods, and compounds for identifying RAS binding compounds using a RAS binding agent, which comprises a RAS binding moiety and a functional element. In some embodiments, the RAS binding agent binds to one site on the RAS protein (e.g., KRAS, HRAS, or NRAS), and can be used to detect RAS binding agents that bind to the same site as well as other sites.

### CROSS-REFERENCE TO RELATED APPLICATIONS

The present application claims priority to and the benefit of U.S. Provisional Patent Application No. 63/071,694, filed Aug. 28, 2020, U.S. Provisional Patent Application No. 63/117,080, filed Nov. 23, 2020, and U.S. Provisional Patent Application No. 63/160,120, filed Mar. 12, 2021, each of which is incorporated herein by reference in its entirety.

### SEQUENCE LISTING

Incorporated by reference in its entirety herein is a computer-readable nucleotide sequence listing submitted herewith and identified as follows: one 54,445 byte ASCII (text) file named “Sequence_Listing_ST25.txt” created on Nov. 11, 2021.

### FIELD

Provided herein are systems, methods, and compounds for identifying RAS binding compounds, including KRAS, HRAS, and NRAS binding compounds. In particular, disclosed herein are RAS binding agents, which comprise a RAS binding moiety and a functional element, and can be used to probe target engagement at a variety of RAS binding sites.

### BACKGROUND

RAS proteins regulate a myriad of signaling cascades involved in a variety of cellular processes. RAS genes are oncogenes and, when mutated, can cause normal cells to become cancerous. RAS genes include KRAS, HRAS, and NRAS, which encode the KRAS, HRAS, and NRAS proteins respectively. These proteins relays signals from outside the cell to the cell's nucleus that instruct the cell to grow, divide, mature, and/or differentiate. RAS proteins are GTPases that act as a molecular switches, turning on and off by the conversion of GTP to GDP.

RAS-activating mutations are the most frequent oncogenic alterations in human cancers. RAS-activating mutations fix the RAS protein in its active GTP-bound form by interfering with the GTP to GDP cycling process thereby driving neoplastic transformation of cells. One common KRAS-activating mutation is KRAS^{G12C}, which is particularly prevalent in non-small cell lung cancer. Common HRAS-activating mutations are HRAS^{G12S} and HRAS^{G12V}; the HRAS^{G12S} mutation is associated with Costello syndrome, while the HRAS^{G12V} is associated with bladder cancer. NRAS mutations, such as NRAS^{G12D} and NRAS^{Q61R}, are associated with a variety of human tumors, such as melanoma.

RAS proteins have historically been considered undruggable. Developing RAS inhibitors has been challenging in part because RAS proteins have extremely high affinities for the GTP substrate. For example, GTP occupies the switch I (SI) site of RAS proteins with extremely high affinity, therefore competitive inhibition in cells is considered nearly impossible. It was recently discovered that an oncogenic variant of KRAS (KRAS G12C) could be inhibited by covalent inhibition at the switch II (SII) site. Currently, there are covalent SII site inhibitors in advanced clinical trials (see, e.g., https://clinicaltrials.gov/ct2/show/NCT03600883). More recently, it was discovered that a shallow binding pocket exists between the SI and SII sites. This pocket, termed the SI/SII site, could provide an opportunity for reversible inhibition (Kessler et al. Proc. Natl. Acad. Sci. USA 116:15823-15829 (2019)). An inhibitor, BI-2852, engages this SI/II site in wild-type KRAS and KRAS mutants, and can inhibit downstream KRAS signaling events in cells.

### SUMMARY

Provided herein are systems, methods, and compounds for identifying RAS binding compounds using a RAS binding agent, which comprises a RAS binding moiety and a functional element. For example, provided herein are systems, methods, and compounds for identifying KRAS binding compounds using a KRAS binding agent, which comprises a KRAS binding moiety and a functional element. The KRAS binding moiety binds to one site on the KRAS protein, yet the systems and methods can successfully interrogate engagement at other KRAS binding sites, thereby enabling a broadly useful live cell target engagement assays to identify KRAS binding compounds (such as KRAS inhibitors) that bind via divergent mechanisms. Also described herein are systems, methods, and compounds for identifying HRAS binding compounds using an HRAS binding agent, which comprises an HRAS binding moiety and a functional element, as well as systems, methods, and compounds for identifying NRAS binding compounds using an NRAS binding agent, which comprises an NRAS binding moiety and a functional element. In some embodiments, a RAS binding agent is a KRAS binding agent, an HRAS binding agent, and/or an NRAS binding agent (i.e., the binding agent may bind to one or all of KRAS, HRAS, and NRAS).

In one aspect, provided herein is a method of identifying a RAS binding compound, the method comprising:

(a) providing a sample comprising a RAS protein; and

(b) contacting the sample with a RAS binding agent comprising a RAS binding moiety and a functional element, and a candidate RAS binding compound.

In some embodiments, provided herein is a method of identifying a KRAS binding compound, the method comprising:

(a) providing a sample comprising a KRAS protein; and

(b) contacting the sample with a KRAS binding agent comprising a KRAS binding moiety and a functional element, and a candidate KRAS binding compound.

In some embodiments, the method further comprises a step of: (c) detecting or quantifying the functional element.

In some embodiments, the KRAS protein is a KRAS variant. In some embodiments, the KRAS variant is KRAS^{G12C}, KRAS^{G12D}, KRAS^{G12V}, KRAS^{Q61R}, KRAS^{Q61H}, KRAS^{Q61L}, or KRAS^{G13D}.

In some embodiments, step (a) comprises expressing the KRAS protein within the sample.

In some embodiments, provided herein is a method of identifying an HRAS binding compound, the method comprising:

(a) providing a sample comprising an HRAS protein; and

(b) contacting the sample with an HRAS binding agent comprising an HRAS binding moiety and a functional element, and a candidate HRAS binding compound.

In some embodiments, the method further comprises a step of: (c) detecting or quantifying the functional element.

In some embodiments, the HRAS protein is an HRAS variant. In some embodiments, the HRAS variant is HRAS^{G12S} or HRAS^{G12V}.

In some embodiments, step (a) comprises expressing the HRAS protein within the sample.

In some embodiments, provided herein is a method of identifying an NRAS binding compound, the method comprising:

(a) providing a sample comprising an NRAS protein; and

(b) contacting the sample with an NRAS binding agent comprising an NRAS binding moiety and a functional element, and a candidate NRAS binding compound.

In some embodiments, the method further comprises a step of: (c) detecting or quantifying the functional element.

In some embodiments, the NRAS protein is an NRAS variant. In some embodiments, the NRAS variant is NRAS^{G12D} or NRAS^{Q61R}.

In some embodiments, step (a) comprises expressing the NRAS protein within the sample.

In some embodiments, the RAS binding agent is a compound of formula (I):

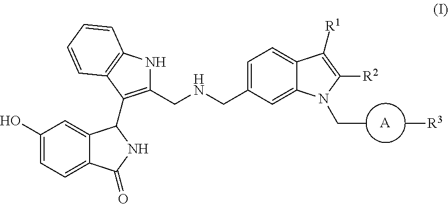

or a salt thereof, wherein:

A is a monocyclic aryl or heteroaryl;

one of R¹, R², and R³ is a group -Linker-B, wherein B is a functional element; and

the other two of R¹, R², and R³ are independently selected from hydrogen and methyl. In some embodiments, A is selected from phenyl, imidazole, pyrrole, pyridyl, thiophene, and triazole. In some embodiments, R¹ is a group -Linker-B, and R² and R³ are independently selected from hydrogen and methyl. In some embodiments, R³ is a group -Linker-B, and R¹ and R² are independently selected from hydrogen and methyl. In some embodiments, Linker has a formula:

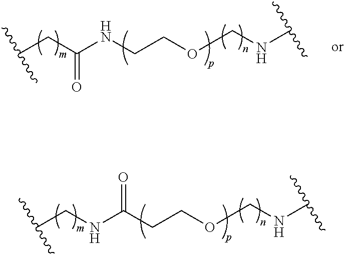

wherein m, n, and p are independently 0, 1, 2, 3, 4, 5, or 6.

In some embodiments, the functional element is a detectable element, an affinity element, a capture element, a solid support, or a moiety that induces protein degradation. In some embodiments, the functional element is a detectable element selected from a fluorophore, chromophore, radionuclide, electron opaque molecule, MRI contrast agent, SPECT contrast agent, and mass tag. In some embodiments, the detectable element, or the signal produced thereby, is detected or quantified by fluorescence, mass spectrometry, optical imaging, radionuclide detection, magnetic resonance imaging (MRI), single photon emission computed tomography (SPECT), or energy transfer. In some embodiments, the functional element is a solid support selected from a sedimental particle, a membrane, glass, a tube, a well, a self-assembled monolayer, a surface plasmon resonance chip, and a solid support with an electron conducting surface. In some embodiments, the sedimental particle is a magnetic particle. In some embodiments, the functional element is a moiety that induces protein degradation. In some embodiments, the functional element is a moiety that induces protein degradation through proteolysis-targeting chimera (PROTAC) tagging. In some embodiments, the detectable element is a fluorophore.

In some embodiments, the candidate RAS binding compound binds the RAS protein. In some embodiments, the candidate RAS binding compound is a RAS inhibitor. In some embodiments, the RAS binding agent binds to the RAS Switch I/II site. In some embodiments, the candidate RAS binding compound binds to the RAS Switch I/II site or to the RAS Switch II site.

In some embodiments, the sample is selected from a cell, cell lysate, body fluid, tissue, biological sample, in vitro sample, environmental sample, cell-free sample, and purified sample (e.g., a purified protein sample).

In some embodiments, the RAS protein is provided as a fusion with a bioluminescent reporter. In some embodiments, the bioluminescent reporter is a luciferase with at least 70% sequence identity with SEQ ID NO: 24. In some embodiments, the sample comprises a first RAS protein fused with a first subunit of a bioluminescent reporter, and a second RAS protein fused with a second subunit of a bioluminescent reporter, wherein the first and second subunits are complementary. In some embodiments, the first subunit of the bioluminescent reporter has at least 70% sequence identity with SEQ ID NO: 25, and the second subunit of the bioluminescent reporter has at least 90% sequence identity with SEQ ID NO: 26. In some embodiments, the emission spectrum of the bioluminescent reporter and the excitation spectrum of the functional element overlap.

In some embodiments, the method further comprises contacting the sample with a substrate for the bioluminescent reporter. In some embodiments, the substrate is coelenterazine, a coelenterazine derivative, or furimazine.

In one aspect, provided herein is a system comprising:

(a) a target RAS protein;

(b) a RAS binding agent comprising a RAS binding moiety and a functional element; and

(c) a candidate RAS binding compound.

In some embodiments, provided herein is a system comprising:

(a) a target KRAS protein;

(b) a KRAS binding agent comprising a KRAS binding moiety and a functional element; and

(c) a candidate KRAS binding compound.

In some embodiments, the target KRAS protein is expressed within the system. In some embodiments, the target KRAS protein is a KRAS variant. In some embodiments, the KRAS variant is selected from KRAS^{G12C}, KRAS^{G12D}, KRAS^{G12V}, KRAS^{Q61R}, KRAS^{Q61H}, KRAS^{Q61L}, and KRAS^{G13D}.

In some embodiments, provided herein is a system comprising:

(a) a target HRAS protein;

(b) an HRAS binding agent comprising an HRAS binding moiety and a functional element; and

(c) a candidate HRAS binding compound.

In some embodiments, the target HRAS protein is expressed within the system. In some embodiments, the HRAS protein is an HRAS variant. In some embodiments, the HRAS variant is HRAS^{G12S} or HRAS^{G12V}.

In some embodiments, provided herein is a system comprising:

(a) a target NRAS protein;

(b) an NRAS binding agent comprising an NRAS binding moiety and a functional element; and

(c) a candidate NRAS binding compound.

In some embodiments, the target NRAS protein is expressed within the system. In some embodiments, the NRAS protein is an NRAS variant. In some embodiments, the NRAS variant is NRAS^{G12D} or NRAS^{Q61R}.

In some embodiments, the RAS binding agent is a compound of formula (I):

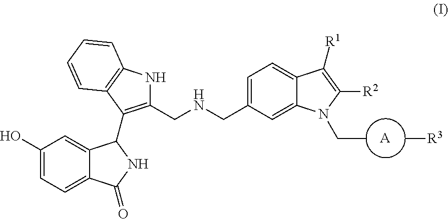

or a salt thereof, wherein:

A is a monocyclic aryl or heteroaryl;

one of R¹, R², and R³ is a group -Linker-B, wherein B is a functional element; and

the other two of R¹, R², and R³ are independently selected from hydrogen and methyl.

In some embodiments, A is selected from phenyl, imidazole, pyrrole, pyridyl, thiophene, and triazole. In some embodiments, R¹ is a group -Linker-B, and R² and R³ are independently selected from hydrogen and methyl. In some embodiments, R³ is a group -Linker-B, and R¹ and R² are independently selected from hydrogen and methyl. In some embodiments, Linker has a formula:

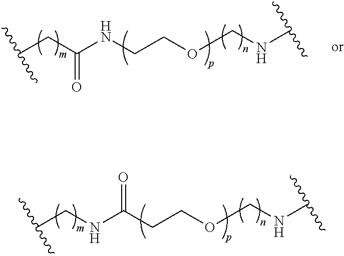

wherein m, n, and p are independently 0, 1, 2, 3, 4, 5, or 6.

In some embodiments, the functional element is a detectable element, an affinity element, a capture element, a solid support, or a moiety that induces protein degradation. In some embodiments, the functional element is a detectable element selected from a fluorophore, chromophore, radionuclide, electron opaque molecule, an MRI contrast agent, SPECT contrast agent, and mass tag. In some embodiments, the detectable element, or the signal produced thereby, is detectable or quantifiable by fluorescence, mass spectrometry, optical imaging, radionuclide detection, magnetic resonance imaging (MRI), single photon emission computed tomography (SPECT), or energy transfer. In some embodiments, the functional element is a solid support selected from a sedimental particle, a membrane, glass, a tube, a well, a self-assembled monolayer, a surface plasmon resonance chip, and a solid support with an electron conducting surface. In some embodiments, the sedimental particle is a magnetic particle. In some embodiments, the functional element is a moiety that induces protein degradation. In some embodiments, the functional element is a moiety that induces protein degradation through proteolysis-targeting chimera (PROTAC) tagging. In some embodiments, the detectable element is a fluorophore.

In some embodiments, the candidate RAS binding compound binds the RAS protein. In some embodiments, the candidate RAS binding compound is a RAS inhibitor.

In some embodiments, the RAS binding moiety binds to the RAS Switch I/II site. In some embodiments, the candidate RAS binding compound binds to the RAS Switch I/II site or to the RAS Switch II site.

In some embodiments, the system comprises a sample selected from a cell, cell lysate, body fluid, tissue, biological sample, in vitro sample, environmental sample, cell-free sample, and purified sample (e.g., a purified protein sample).

In some embodiments, the target RAS protein is present as a fusion with a bioluminescent reporter. In some embodiments, the bioluminescent reporter is a luciferase with at least 70% sequence identity with SEQ ID NO: 24. In some embodiments, the target RAS protein comprises a first RAS protein fused with a first subunit of a bioluminescent reporter, and a second RAS protein fused with a second subunit of a bioluminescent reporter, wherein the first and second subunits are complementary. In some embodiments, the first subunit of the bioluminescent reporter has at least 70% sequence identity with SEQ ID NO: 25, and the second subunit of the bioluminescent reporter has at least 70% sequence identity with SEQ ID NO: 26. In some embodiments, the emission spectrum of the bioluminescent reporter and the excitation spectrum of the functional element overlap.

In some embodiments, the system further comprises a substrate for the bioluminescent reporter. In some embodiments, the substrate is coelenterazine, a coelenterazine derivative, or furimazine.

In one aspect, provided herein is a RAS binding agent comprising:

(a) a RAS binding moiety; and

(b) a functional element.

In some embodiments, the RAS binding agent is a KRAS binding agent comprising:

(a) a KRAS binding moiety; and

(b) a functional element.

In some embodiments, the RAS binding agent is an HRAS binding agent comprising:

(a) an HRAS binding moiety; and

(b) a functional element.

In some embodiments, the RAS binding agent is an NRAS binding agent comprising:

(a) an NRAS binding moiety; and

(b) a functional element.

In some embodiments, the RAS binding agent further comprises a linker connecting the RAS binding moiety and the functional element.

In some embodiments, the RAS binding agent is a compound of formula (I):

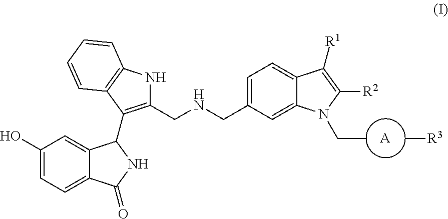

or a salt thereof, wherein:

A is a monocyclic aryl or heteroaryl;

one of R¹, R², and R³ is a group -Linker-B, wherein B is a functional element; and

the other two of R¹, R², and R³ are independently selected from hydrogen and methyl.

In some embodiments, A is selected from phenyl, imidazole, pyrrole, pyridyl, thiophene, and triazole. In some embodiments, R¹ is a group -Linker-B, and R² and R³ are independently selected from hydrogen and methyl. In some embodiments, R³ is a group -Linker-B, and R¹ and R² are independently selected from hydrogen and methyl. In some embodiments, Linker has a formula:

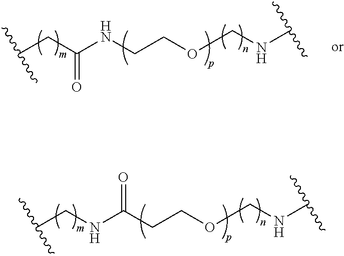

wherein m, n, and p are independently 0, 1, 2, 3, 4, 5, or 6.

In some embodiments, the functional element is a detectable element, an affinity element, a capture element, a solid support, or a moiety that induces protein degradation. In some embodiments, the functional element is a detectable element selected from a fluorophore, chromophore, radionuclide, electron opaque molecule, an MRI contrast agent, SPECT contrast agent, and mass tag. In some embodiments, the detectable element is a fluorophore. In some embodiments, the detectable element, or the signal produced thereby, is detected or quantified by fluorescence, mass spectrometry, optical imaging, radionuclide detection, magnetic resonance imaging (MRI), single photon emission computed tomography (SPECT), or energy transfer. In some embodiments, the functional element is a solid support selected from a sedimental particle, a membrane, glass, a tube, a well, a self-assembled monolayer, a surface plasmon resonance chip, and a solid support with an electron conducting surface. In some embodiments, the sedimental particle is a magnetic particle. In some embodiments, the functional element is a moiety that induces protein degradation. In some embodiments, the functional element is a moiety that induces protein degradation through proteolysis-targeting chimera (PROTAC) tagging.

In some embodiments, the RAS binding moiety binds to the RAS Switch I/II site.

In one aspect, disclosed herein is a composition comprising a RAS binding agent described herein (e.g., a RAS binding agent comprising a RAS binding moiety and a functional element, such as a compound of formula (I)).

In some embodiments, the composition further comprises a RAS protein. In some embodiments, the RAS protein is selected from a KRAS protein, an HRAS protein, and an NRAS protein. In some embodiments, the RAS protein is present as a fusion with a bioluminescent reporter. In some embodiments, the bioluminescent reporter is a luciferase with at least 70% sequence identity with SEQ ID NO: 24. In some embodiments, the composition comprises a first RAS protein fused with a first subunit of a bioluminescent reporter, and a second RAS protein fused with a second subunit of a bioluminescent reporter, wherein the first and second subunits are complementary. In some embodiments, the first subunit of the bioluminescent reporter has at least 70% sequence identity with SEQ ID NO: 25, and the second subunit of the bioluminescent reporter has at least 70% sequence identity with SEQ ID NO: 26. In some embodiments, the emission spectrum of the bioluminescent reporter and the excitation spectrum of the functional element overlap.

In some embodiments, the composition further comprises a substrate for the bioluminescent reporter. In some embodiments, the substrate is coelenterazine, a coelenterazine derivative, or furimazine.

In some embodiments, the composition further comprises a candidate RAS binding compound. In some embodiments, the candidate RAS binding compound is a candidate KRAS binding compound, a candidate HRAS binding compound, or a candidate NRAS binding compound. In some embodiments, the candidate RAS binding compound is a RAS inhibitor. In some embodiments, the candidate RAS binding compound binds to the RAS Switch I/II site or to the RAS Switch II site.

In one aspect, provided herein is a method for screening for a RAS binding compound, the method comprising:

(a) contacting a sample comprising: (i) a RAS protein; and (ii) a RAS binding agent comprising a RAS binding moiety and a functional element, with a candidate RAS binding compound; and

(b) detecting or quantifying a signal from the functional element.

In some embodiments, provided herein is a method for screening for a KRAS binding compound, the method comprising:

(a) contacting a sample comprising: (i) a KRAS protein; and (ii) a KRAS binding agent comprising a KRAS binding moiety and a functional element, with a candidate KRAS binding compound; and

(b) detecting or quantifying a signal from the functional element.

In some embodiments, the KRAS protein is a KRAS variant. In some embodiments, the KRAS variant is selected from KRAS^{G12C}, KRAS^{G12D}, KRAS^{G12V}, KRAS^{Q61R}, KRAS^{Q61H}, KRAS^{Q61L}, and KRAS^{G13D}.

In some embodiments, provided herein is a method for screening for an HRAS binding compound, the method comprising:

(a) contacting a sample comprising: (i) an HRAS protein; and (ii) an HRAS binding agent comprising an HRAS binding moiety and a functional element, with a candidate HRAS binding compound; and

(b) detecting or quantifying a signal from the functional element.

In some embodiments, the HRAS protein is an HRAS variant. In some embodiments, the HRAS variant is HRAS^{G12S} or HRAS^{G12V}.

In some embodiments, provided herein is a method for screening for an NRAS binding compound, the method comprising:

(a) contacting a sample comprising: (i) an NRAS protein; and (ii) an NRAS binding agent comprising an NRAS binding moiety and a functional element, with a candidate NRAS binding compound; and

(b) detecting or quantifying a signal from the functional element.

In some embodiments, the NRAS protein is an NRAS variant. In some embodiments, the NRAS variant is NRAS^{G12D} or NRAS^{Q61R}.

In some embodiments, the candidate RAS binding compound binds the RAS protein and detectably alters the signal from the functional element.

In some embodiments, the candidate RAS binding compound is a RAS inhibitor. In some embodiments, the candidate RAS binding compound binds to the RAS Switch I/II site or to the RAS Switch II site.

In some embodiments, the RAS binding agent is a compound of formula (I):

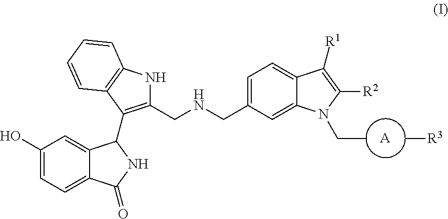

or a salt thereof, wherein:

A is a monocyclic aryl or heteroaryl;

one of R¹, R², and R³ is a group -Linker-B, wherein B is a functional element; and

the other two of R¹, R², and R³ are independently selected from hydrogen and methyl.

In some embodiments, A is selected from phenyl, imidazole, pyrrole, pyridyl, thiophene, and triazole. In some embodiments, R¹ is a group -Linker-B, and R² and R³ are independently selected from hydrogen and methyl. In some embodiments, R³ is a group -Linker-B, and R¹ and R² are independently selected from hydrogen and methyl. In some embodiments, Linker has a formula:

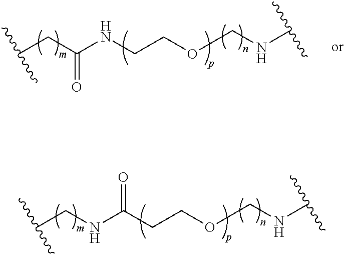

wherein m, n, and p are independently 0, 1, 2, 3, 4, 5, or 6.

In some embodiments, the functional element is a detectable element, an affinity element, a capture element, a solid support, or a moiety that induces protein degradation. In some embodiments, the functional element is a detectable element selected from a fluorophore, chromophore, radionuclide, electron opaque molecule, an MRI contrast agent, SPECT contrast agent, and mass tag. In some embodiments, the detectable element, or the signal produced thereby, is detected or quantified by fluorescence, mass spectrometry, optical imaging, radionuclide detection, magnetic resonance imaging (MRI), single photon emission computed tomography (SPECT), or energy transfer. In some embodiments, the functional element is a moiety that induces protein degradation. In some embodiments, the functional element is a moiety that induces protein degradation through proteolysis-targeting chimera (PROTAC) tagging. In some embodiments, the detectable element is a fluorophore.

In some embodiments, the RAS binding moiety binds to the RAS Switch I/II site.

In some embodiments, the RAS protein is present as a fusion with a bioluminescent reporter. In some embodiments, the bioluminescent reporter is a luciferase with at least 70% sequence identity with SEQ ID NO: 24. In some embodiments, the RAS protein comprises a first RAS protein fused with a first subunit of a bioluminescent reporter, and a second RAS protein fused with a second subunit of a bioluminescent reporter, wherein the first and second subunits are complementary. In some embodiments, the first subunit of the bioluminescent reporter has at least 70% sequence identity with SEQ ID NO: 25, and the second subunit of the bioluminescent reporter has at least 70% sequence identity with SEQ ID NO: 26.

In some embodiments, the emission spectrum of the bioluminescent reporter and the excitation spectrum of the functional element overlap.

In some embodiments, the composition further comprises a substrate for the bioluminescent reporter. In some embodiments, the substrate is coelenterazine, a coelenterazine derivative, or furimazine.

### BRIEF DESCRIPTION OF THE DRAWINGS

FIG. 1 shows an illustration of an exemplary target engagement assay according to the present disclosure using a RAS-NLuc fusion and a RAS binding agent comprising a RAS binding moiety and an energy acceptor.

FIG. 2 shows data from a NanoBiT assay in cells expressing KRAS or a variant thereof (KRAS^{G12C}, KRAS^{G12D}, or KRAS^{G12V}) as fusions with LgBiT and SmBiT. The data demonstrates competition between a KRAS binding agent disclosed herein (compound JRW-2111) and the compound BI-2852.

FIG. 3 shows data from a NanoBiT assay in cells expressing KRAS or a variant thereof (KRAS^{G12C}, KRAS^{G12D}, or KRAS^{G12V}) as fusions with LgBiT and SmBiT. The data demonstrates competition between a KRAS binding agent disclosed herein (compound JRW-2111) and the compound AMG-510 only for the KRAS^{G12C} variant, but not for wild-type KRAS or the other two KRAS variants.

FIG. 4 shows data from cells expressing KRAS or a variant thereof (KRAS^{G12C}, KRAS^{G12D}, or KRAS^{G12V}) as a fusion with NanoLuc. The data demonstrates competition between a KRAS binding agent disclosed herein (compound JRW-2025) and compound BI-2852 for wild-type KRAS and the three KRAS variants, but competition was only observed for the KRAS^{G12C} variant with compounds AMG-510 and ARS-1620.

FIG. 5 shows data from cells expressing KRAS-NanoBiT fusion proteins (LgBiT-KRAS2B^{G12V} and SmBiT-KRAS2B^{G12V}) and treated with a KRAS binding agent disclosed herein (compound JRW-2192) and compound BI-2852.

FIG. 6 shows data from cells expressing KRAS-NanoBiT fusion proteins (LgBiT-KRAS2B^{G12C} and SmBiT-KRAS2B^{G12C}) and treated with a KRAS binding agent disclosed herein (compound JRW-2220) and compound BI-2852.

FIG. 7 shows data from digitonin-permeabilized cells expressing KRAS-NanoBiT fusion proteins (LgBiT-KRAS2B^{G12C} and SmBiT-KRAS2B^{G12C}) and treated with a KRAS binding agent disclosed herein (compound JRW-2220) and compound BI-2852.

FIG. 8 shows data from cells expressing KRAS-NanoBiT fusions (LgBiT-KRAS2B^{G12C} and HiBiT-KRAS2B^{G12C}) and treated with a KRAS binding agent disclosed herein (compound JRW-2220) and compound BI-2852.

FIG. 9 shows data from digitonin-permeabilized cells expressing KRAS-NanoBiT fusions (LgBiT-KRAS2B^{G12C} and HiBiT-KRAS2B^{G12C}) and treated with a KRAS binding agent disclosed herein (compound JRW-2220) and compound BI-2852.

FIG. 10 shows data from cells expressing KRAS-NanoBiT fusions (LgBiT-KRAS2B and SmBiT-KRAS2B) and treated with a KRAS binding agent disclosed herein (compound JRW-2219) and compound BI-2852.

FIG. 11 shows data from cells expressing KRAS-NanoBiT fusions (LgBiT-KRAS2B and SmBiT-KRAS2B) and treated with a KRAS binding agent disclosed herein (compound JRW-2219) and compound BI-2852.

FIG. 12 shows data from cells expressing KRAS-NanoBiT fusions (LgBiT-KRAS2B^{G12C} and SmBiT-KRAS2B^{G12C}) and treated with a KRAS binding agent disclosed herein (compound JRW-2219) and compound BI-2852.

FIG. 13 shows data from cells expressing KRAS-NanoBiT fusions (LgBiT-KRAS2B^{G12C} and SmBiT-KRAS2B^{G12C}) and treated with a KRAS binding agent disclosed herein (compound JRW-2220) and compound BI-2852.

FIG. 14 shows data from cells expressing KRAS-NanoBiT fusions (LgBiT-KRAS2B^{G12D} and SmBiT-KRAS2B^{G12D}) and treated with a KRAS binding agent disclosed herein (compound JRW-2219) and compound BI-2852.

FIG. 15 shows data from cells expressing KRAS-NanoBiT fusions (LgBiT-KRAS2B^{G12D} and SmBiT-KRAS2B^{G12D}) and treated with a KRAS binding agent disclosed herein (compound JRW-2220) and compound BI-2852.

FIG. 16 shows data from cells expressing KRAS-NanoBiT fusions (LgBiT-KRAS2B^{G12V} and SmBiT-KRAS2B^{G12V}) and treated with a KRAS binding agent disclosed herein (compound JRW-2219) and compound BI-2852.

FIG. 17 shows data from cells expressing KRAS-NanoBiT fusions (LgBiT-KRAS2B^{G12V} and SmBiT-KRAS2B^{G12V}) and treated with a KRAS binding agent disclosed herein (compound JRW-2220) and compound BI-2852.

FIG. 18 shows data from cells expressing HRAS-NanoBiT fusions (LgBiT-HRAS1 and SmBiT-HRAS1) and treated with a RAS binding agent disclosed herein (compound JRW-2219) and compound BI-2852.

FIG. 19 shows data from cells expressing HRAS-NanoBiT fusions (LgBiT-HRAS1 and SmBiT-HRAS1) and treated with a RAS binding agent disclosed herein (compound JRW-2220) and compound BI-2852.

FIG. 20 shows data from cells expressing KRAS-NanoBiT fusions and treated with RAS binding agents disclosed herein (compounds JRW-2220 and JRW-2310) and compound BI-2582.

FIG. 21 shows data from cells expressing KRAS-NanoBiT fusions (LgBiT-KRAS2B^{Q61R} and SmBiT-KRAS2B^{Q61R}) and treated with a RAS binding agent disclosed herein (compound JRW-2310) and compound BI-2852.

FIG. 22 shows data from cells expressing KRAS-NanoBiT fusions (LgBiT-KRAS2B^{Q61H} and SmBiT-KRAS2B^{Q61H}) and treated with a RAS binding agent disclosed herein (compound JRW-2310) and compound BI-2852.

FIG. 23 shows data from cells expressing KRAS-NanoBiT fusions (LgBiT-KRAS2B^{Q61L} and SmBiT-KRAS2B^{Q61L}) and treated with a RAS binding agent disclosed herein (compound JRW-2310) and compound BI-2852.

FIG. 24 shows data from cells expressing KRAS-NanoBiT fusions (LgBiT-KRAS2B^{QG13D} and SmBiT-KRAS2B^{G13D}) and treated with a RAS binding agent disclosed herein (compound JRW-2310) and compound BI-2852.

FIG. 25 shows data from cells expressing NRAS-NanoBiT fusions (LgBiT-NRAS and SmBiT-NRAS) and treated with a RAS binding agent disclosed herein (compound JRW-2310) and compound BI-2852.

### DETAILED DESCRIPTION

Provided herein are systems, methods, and compositions for identifying RAS binding compounds such as RAS inhibitors or modulators. RAS proteins include NRAS, HRAS, and KRAS (including isoforms, KRAS4A and KRAS4B). In particular, provided herein are systems, methods, and compositions for identifying RAS binding compounds such as RAS inhibitors or modulators. The systems and methods include a RAS binding agent, which comprises a RAS binding moiety and a functional element. The methods involve providing a sample comprising a RAS protein and contacting the sample with the RAS binding agent and a candidate RAS binding compound. In some embodiments, the methods further comprise a step of detecting or quantifying the functional element, e.g., by detecting a signal from the functional element. The methods, systems, and compounds can be used to measure target engagement by RAS proteins not only at the site at which the RAS binding agent binds, but also at other RAS binding sites. For example, in some embodiments, the RAS binding agent binds at the RAS switch I/II site, and the systems and methods can be used to probe target engagement not only at the switch I/II site but also at other sites, such as the switch II site.

### I. DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols as herein described as these may vary in accordance with routine experimentation and optimization. It is also to be understood that the terminology used in the description is for the purpose of describing the particular versions or embodiments only and is not intended to limit the scope of the embodiments described herein.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein and in the appended claims, the singular forms “a”, “an”, and “the” include plural reference unless the context clearly dictates otherwise. Thus, for example, reference to “a peptide” is a reference to one or more peptides and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.” As used herein, the term “comprise” and linguistic variations thereof denote the presence of recited feature(s), element(s), method step(s), etc., without the exclusion of the presence of additional feature(s), element(s), method step(s), etc. Conversely, the term “consisting of” and linguistic variations thereof, denotes the presence of recited feature(s), element(s), method step(s), etc., and excludes any unrecited feature(s), element(s), method step(s), etc., except for ordinarily-associated impurities. The phrase “consisting essentially of” denotes the recited feature(s), element(s), method step(s), etc., and any additional feature(s), element(s), method step(s), etc., that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “affinity element” refers to a molecular entity that forms a stable noncovalent interaction with a corresponding “affinity agent.” As used herein, the term “capture element” refers to a molecular entity that forms a

covalent interaction with a corresponding “capture agent.” As used herein, the term “detectable element” refers to a detectable, reactive, affinity, or otherwise bioactive agent or moiety that is attached (e.g., directly or via a suitable linker) to a compound described herein (or derivatives or analogs thereof, etc.). Other additional detectable elements that may find use in embodiments described herein comprise “localization elements”, “detection elements”, etc.

“Coelenterazine” as used herein refers to naturally-occurring (“native”) coelenterazine. As used herein, the term “coelenterazine analog” or “coelenterazine derivative” refers to synthetic (e.g., derivative or variant) and natural analogs thereof, including furimazine, coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine (“coelenterazine-hh”), coelenterazine-i, coelenterazine-icp, coelenterazine-v, and 2-methyl coelenterazine, in addition to those disclosed in U.S. Pat. Pub. No. 2008/0248511; U.S. Pat. Pub. No. 2012/0174242; U.S. Pat. Pub. 2017/0233789; and U.S. Pat. Pub. 2018/0030059; the disclosures of each of which are incorporated herein by reference herein in their entireties. In some embodiments, coelenterazine analogs include pro-substrates such as, for example, those described in U.S. Pat. Pub. No. 2008/0248511; U.S. Pub. No. 2012/0707849; and U.S. Pub. No. 2014/0099654; the disclosures of each of which are incorporated herein by reference herein in their entireties.

As used herein, the term “energy acceptor” refers to any small molecule (e.g., chromophore), macromolecule (e.g., autofluorescent protein, phycobiliprotein, nanoparticle, surface, etc.), or molecular complex that produces a readily detectable signal in response to energy absorption (e.g., resonance energy transfer). In certain embodiments, an energy acceptor is a fluorophore or other detectable chromophore.

As used herein, the term “RAS Switch I site” refers to a site on the RAS protein spanning residues 30-38, and the term “RAS Switch II site” refers to a site on the RAS protein spanning residues 60-76 as disclosed by Milburn et al. (Science 247:939-945 (1990)) and Kessler et al. (Proc. Natl. Acad. Sci. USA 116:15823-15829 (2019)), each of which is incorporated herein by reference in its entirety. As used herein, the term “RAS Switch I/II site” refers to a pocket between the RAS Switch I site and the RAS Switch II site as disclosed by Kessler et al. (Proc. Natl. Acad. Sci. USA 116:15823-15829 (2019).

“Peptide” and “polypeptide” as used herein, and unless otherwise specified, refer to polymer compounds of two or more amino acids joined through the main chain by peptide amide bonds (—C(O)NH—). The term “peptide” typically refers to short amino acid polymers (e.g., chains having fewer than 25 amino acids), whereas the term “polypeptide” typically refers to longer amino acid polymers (e.g., chains having more than 25 amino acids).

As used herein, the term “sample” is used in its broadest sense. In one sense, it is meant to include a specimen or culture obtained from any source as well as biological and environmental samples. Biological samples may be obtained from animals (including humans) and encompass fluids, solids, tissues, and gases. Biological samples include blood products such as plasma, serum, and the like. Sample may also refer to cells, cell lysates or purified forms of the enzymes, peptides, and/or polypeptides described herein (e.g., a purified protein sample). Cell lysates may include cells that have been lysed with a lysing agent or lysates such as rabbit reticulocyte or wheat germ lysates. Sample may also include in vitro samples and cell-free samples, such as cell-free expression systems. Environmental samples include environmental material such as surface matter, soil, water, crystals, and industrial samples. Sample may also include purified samples, such as purified protein samples. Such examples are not however to be construed as limiting the sample types applicable to the present invention.

As used herein, the term “solid support” is used in reference to any solid or stationary material to which reagents such as substrates, mutant proteins, drug-like molecules, and other test components are or may be attached. Examples of solid supports include microscope slides, wells of microtiter plates, coverslips, beads, particles, resin, cell culture flasks, as well as many other suitable items. The beads, particles, or resin can be magnetic or paramagnetic.

“Variant” is used herein to describe a peptide or polypeptide that differs in amino acid sequence by the insertion, deletion, or conservative substitution of amino acids, but retains at least one biological activity. “SNP” refers to a variant that is a single nucleotide polymorphism. Representative examples of “biological activity” include the ability to be bound by a specific antibody or to promote an immune response. Variant is also used herein to describe a protein with an amino acid sequence that is substantially identical to a referenced protein with an amino acid sequence that retains at least one biological activity. A conservative substitution of an amino acid (e.g., replacing an amino acid with a different amino acid of similar properties, such as hydrophilicity, degree, and distribution of charged regions) is recognized in the art as typically involving a minor change. These minor changes can be identified, in part, by considering the hydropathic index of amino acids, as understood in the art. The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; and Carruthers, Some Modern Methods of Organic Synthesis, 3^{rd} Edition, Cambridge University Press, Cambridge, 1987; the entire contents of each of which are incorporated herein by reference.

As used herein, the term “alkyl” means a straight or branched saturated hydrocarbon chain containing from 1 to 30 carbon atoms, for example 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), 1 to 4 carbon atoms (C₁-C₄ alkyl), 6 to 20 carbon atoms (C₆-C₂₀ alkyl), or 8 to 14 carbon atoms (C₈-C₁₄ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “alkylene” refers to a divalent group derived from a straight or branched chain hydrocarbon of 1 to 12 carbon atoms (C₁-C₁₂ alkylene), for example, of 1 to 6 carbon atoms (C₁-C₆ alkylene). Representative examples of alkylene include, but are not limited to, —CH₂—, —CH₂CH₂—, —CH(CH₃)—, —CH₂CH₂CH₂—, —CH₂CH(CH₃)—, —CH₂CH₂CH₂CH₂—, —CH₂CH(CH₃)CH₂—, —CH₂CH₂CH(CH₃)—, —CH₂CH₂CH₂CH₂CH₂—, —CH₂CH(CH₃)CH₂CH₂—, —CH(CH₃)CH₂CH₂CH₂—, —CH₂CH₂CH₂CH₂CH₂CH₂—, —CH₂CH₂CH(CH₃)CH₂CH₂—, —CH₂CH(CH₃)CH₂CH₂CH₂—, and —CH(CH₃)CH₂CH₂CH₂CH₂—.

As used herein, the term “alkenyl” refers to a straight or branched hydrocarbon chain containing from 2 to 30 carbon atoms and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

As used herein, the term “alkynyl” refers to a straight or branched hydrocarbon chain containing from 2 to 30 carbon atoms and containing at least one carbon-carbon triple bond. Representative examples of alkynyl include, but are not limited to, ethynyl, propynyl, and butynyl.

As used herein, the term “aryl” refers to an aromatic carbocyclic ring system having a single ring (monocyclic) or multiple rings (bicyclic or tricyclic) including fused ring systems, and zero heteroatoms. As used herein, aryl contains 6-20 carbon atoms (C₆-C₂₀ aryl), 6 to 14 ring carbon atoms (C₆-C₁₄ aryl), 6 to 12 ring carbon atoms (C₆-C₁₂ aryl), or 6 to 10 ring carbon atoms (C₆-C₁₀ aryl). Representative examples of aryl groups include, but are not limited to, phenyl, naphthyl, anthracenyl, and phenanthrenyl.

As used herein, the term “arylene” refers to a divalent aryl group. Representative examples of arylene groups include, but are not limited to, phenylene groups (e.g., 1,2-phenylene, 1,3-phenylene, and 1,4-phenylene).

As used herein, the term “cycloalkyl” refers to a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, bicyclo[2.2.1]heptanyl, bicyclo[3.2.1]octanyl, and bicyclo[5.2.0]nonanyl.

As used herein, the term “halogen” or “halo” means F, Cl, Br, or I.

As used herein, the term “haloalkyl” means an alkyl group, as defined herein, in which at least one hydrogen atom (e.g., one, two, three, four, five, six, seven or eight hydrogen atoms) is replaced by a halogen.

As used herein, the term “heteroalkyl” means an alkyl group, as defined herein, in which one or more of the carbon atoms (and any associated hydrogen atoms) are each independently replaced with a heteroatom group such as —NR—, —O—, —S—, —S(O)—, —S(O)₂—, and the like, where R is H, alkyl, aryl, cycloalkyl, heteroalkyl, heteroaryl, or heterocyclyl, each of which may be optionally substituted. By way of example, 1, 2, or 3 carbon atoms may be independently replaced with the same or different heteroatomic group. Examples of heteroalkyl groups include, but are not limited to, —OCH₃, —CH₂OCH₃, —SCH₃, —CH₂SCH₃, —NRCH₃, and —CH₂NRCH₃, where R is hydrogen, alkyl, aryl, arylalkyl, heteroalkyl, or heteroaryl, each of which may be optionally substituted. Heteroalkyl also includes groups in which a carbon atom of the alkyl is oxidized (i.e., is —C(O)—).

As used herein, the term “heteroalkylene” means an alkylene group, as defined herein, in which one or more of the carbon atoms (and any associated hydrogen atoms) are each independently replaced with a heteroatom group such as —NR—, —O—, —S—, —S(O)—, —S(O)₂—, and the like, where R is H, alkyl, aryl, cycloalkyl, heteroalkyl, heteroaryl, or heterocyclyl, each of which may be optionally substituted. By way of example, 1, 2, or 3 carbon atoms may be independently replaced with the same or different heteroatomic group. Heteroalkylene also includes groups in which a carbon atom of the alkyl is oxidized (i.e., is —C(O)—). Examples of heteroalkylene groups include, but are not limited to, —CH₂—O—CH₂—, —CH₂—S—CH₂—, —CH₂—NR—CH₂—, —CH₂—NH—C(O)—CH₂—, and the like, as well as polyethylene oxide chains, polypropylene oxide chains, and polyethyleneimine chains.

As used herein, the term “heteroaryl” refers to an aromatic group having a single ring (monocyclic) or multiple rings (bicyclic or tricyclic) having one or more ring heteroatoms independently selected from O, N, and S. The aromatic monocyclic rings are five- or six-membered rings containing at least one heteroatom independently selected from O, N, and S (e.g. 1, 2, 3, or 4 heteroatoms independently selected from O, N, and S). The five-membered aromatic monocyclic rings have two double bonds, and the six-membered aromatic monocyclic rings have three double bonds. The bicyclic heteroaryl groups are exemplified by a monocyclic heteroaryl ring appended fused to a monocyclic aryl group, as defined herein, or a monocyclic heteroaryl group, as defined herein. The tricyclic heteroaryl groups are exemplified by a monocyclic heteroaryl ring fused to two rings independently selected from a monocyclic aryl group, as defined herein, and a monocyclic heteroaryl group as defined herein. Representative examples of monocyclic heteroaryl include, but are not limited to, pyridinyl (including pyridin-2-yl, pyridin-3-yl, pyridin-4-yl), pyrimidinyl, pyrazinyl, pyridazinyl, pyrrolyl, benzopyrazolyl, 1,2,3-triazolyl, 1,3,4-thiadiazolyl, 1,2,4-thiadiazolyl, 1,3,4-oxadiazolyl, 1,2,4-oxadiazolyl, imidazolyl, thiazolyl, isothiazolyl, thienyl, furanyl, oxazolyl, isoxazolyl, 1,2,4-triazinyl, and 1,3,5-triazinyl. Representative examples of bicyclic heteroaryl include, but are not limited to, benzimidazolyl, benzodioxolyl, benzofuranyl, benzooxadiazolyl, benzopyrazolyl, benzothiazolyl, benzothienyl, benzotriazolyl, benzoxadiazolyl, benzoxazolyl, chromenyl, imidazopyridine, imidazothiazolyl, indazolyl, indolyl, isobenzofuranyl, isoindolyl, isoquinolinyl, naphthyridinyl, purinyl, pyridoimidazolyl, quinazolinyl, quinolinyl, quinoxalinyl, thiazolopyridinyl, thiazolopyrimidinyl, thienopyrrolyl, and thienothienyl. Representative examples of tricyclic heteroaryl include, but are not limited to, dibenzofuranyl and dibenzothienyl. The monocyclic, bicyclic, and tricyclic heteroaryls are connected to the parent molecular moiety through any carbon atom or any nitrogen atom contained within the rings.

As used herein, the term “heterocycle” or “heterocyclic” refers to a saturated or partially unsaturated non-aromatic cyclic group having one or more ring heteroatoms independently selected from O, N, and S. means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from O, N, and S. The three- or four-membered ring contains zero or one double bond, and one heteroatom selected from O, N, and S. The five-membered ring contains zero or one double bond and one, two or three heteroatoms selected from O, N, and S. The six-membered ring contains zero, one, or two double bonds and one, two, or three heteroatoms selected from O, N, and S. The seven-and eight-membered rings contains zero, one, two, or three double bonds and one, two, or three heteroatoms selected from O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, 2-azaspiro[3.3]heptan-2-yl, azabicyclo[2.2.1]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan,hexahydro-1H-1,4-methanocyclopenta[c]furan, aza-adamantane (1-azatricyclo[3.3.1.1^{3,7}]decane), and oxa-adamantane (2-oxatricyclo[3.3.1.1^{3,7}]decane). The monocyclic, bicyclic, and tricyclic heterocycles are connected to the parent molecular moiety through any carbon atom or any nitrogen atom contained within the rings.

As used herein, the term “hydroxy” means an —OH group.

In some instances, the number of carbon atoms in a group (e.g., alkyl, alkoxy, or cycloalkyl) is indicated by the prefix “Cₓ-C_{y}-”, wherein x is the minimum and y is the maximum number of carbon atoms in the group. Thus, for example, “C1-C₃-alkyl” refers to an alkyl group containing from 1 to 3 carbon atoms (i.e. 1, 2, or 3 carbon atoms).

As used herein, the term “substituent” refers to a group substituted on an atom of the indicated group.

When a group or moiety can be substituted, the term “substituted” indicates that one or more (e.g., 1, 2, 3, 4, 5, or 6; in some embodiments 1, 2, or 3; and in other embodiments 1 or 2) hydrogens on the group indicated in the expression using “substituted” can be replaced with a selection of recited indicated groups or with a suitable substituent group known to those of skill in the art (e.g., one or more of the groups recited below), provided that the designated atom's normal valence is not exceeded. Substituent groups include, but are not limited to, alkyl, alkenyl, alkynyl, alkoxy, acyl, amino, amido, amidino, aryl, azido, carbamoyl, carboxyl, carboxyl ester, cyano, cycloalkyl, cycloalkenyl, guanidino, halo, haloalkyl, haloalkoxy, heteroalkyl, heteroaryl, heterocyclyl, hydroxy, hydrazino, imino, oxo, nitro, phosphate, phosphonate, sulfonic acid, thiol, thione, or combinations thereof.

As used herein, in chemical structures the indication:

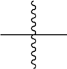

represents a point of attachment of one moiety to another moiety (e.g., a linker to a RAS binding moiety).

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

Where substituent groups are specified by their conventional chemical formulae, written from left to right, they optionally encompass substituents resulting from writing the structure from right to left, e.g., —CH₂O— optionally also recites —OCH₂—, and —OC(O)NH— also optionally recites —NHC(O)O—.

### II. RAS PROTEINS

The methods and systems disclosed herein involve a target RAS protein. In some embodiments, the target RAS protein is a target KRAS protein. The KRAS gene has two splice variants or isoforms: KRAS4A (Accession: NP_001356715.1) and KRAS4B (Accession: NP_004976.2). KRAS4A and KRAS4B are identical in their first 150 amino acid residues and both can be subject to some of the same oncogenic mutations, e.g., at position 12. In some embodiments, the target RAS protein is a target HRAS protein. The HRAS gene also has two splice variants or isoforms: isoform 1 (Accession: NP_001123914.1) and isoform 2 (NP_789765.1). In some embodiments, the target RAS protein is a target NRAS protein (Accession: NP_002515.1).

In some embodiments, the KRAS protein is the wild-type KRAS4A protein (SEQ ID NO: 2). In some embodiments, the KRAS protein is a KRAS4A variant, for example, a variant comprising at least 70% (e.g., 70%, 75%, 80%, 85%, 90%, 95%, 96%, 97%, 98%, 99%, 100%, or ranges therebetween) sequence identity with SEQ ID NO: 2. In some embodiments, the KRAS4A variant is an active variant (e.g., constitutively active) comprising at least 70% (e.g., 70%, 75%, 80%, 85%, 90%, 95%, 96%, 97%, 98%, 99%, 100%, or ranges therebetween) sequence identity with SEQ ID NO: 2.

In some embodiments, the KRAS protein is the wild-type KRAS4B protein (SEQ ID NO: 4). In some embodiments, the KRAS protein is a KRAS4B variant, for example, a variant comprising at least 70% (e.g., 70%, 75%, 80%, 85%, 90%, 95%, 96%, 97%, 98%, 99%, 100%, or ranges therebetween) sequence identity with SEQ ID NO: 4. In some embodiments, the KRAS4B variant is an active variant (e.g., constitutively active) comprising at least 70% (e.g., 70%, 75%, 80%, 85%, 90%, 95%, 96%, 97%, 98%, 99%, 100%, or ranges therebetween) sequence identity with SEQ ID NO: 4.

In some embodiments, the KRAS protein is a KRAS variant selected from KRAS^{G12C}(SEQ ID NO: 5 or SEQ ID NO: 8), KRAS^{G12D} (SEQ ID NO: 6 or SEQ ID NO: 9), KRAS^{G12V}(SEQ ID NO: 7 or SEQ ID NO: 10), KRAS^{Q61R} (SEQ ID NO: 37 or SEQ ID NO: 41), KRAS^{Q61H}, (SEQ ID NO: 38 or SEQ ID NO: 42), KRAS^{Q61L} (SEQ ID NO: 39 or SEQ ID NO: 43), and KRAS^{G13D} (SEQ ID NO: 40 or SEQ ID NO: 44). In some embodiments, the KRAS protein is a KRAS variant with one or more substitutions (e.g., 1, 2, 3, 4, 5, 6, 7, 8, 9, 10, 15, 20, 25, 30, 40, 50, 60, or ranges therebetween) relative to SEQ ID NO: 5, SEQ ID NO: 6, SEQ ID NO: 7, SEQ ID NO: 8, SEQ ID NO: 9, SEQ ID NO: 10, SEQ ID NO: 37, SEQ ID NO: 38, SEQ ID NO: 39, SEQ ID NO: 40, SEQ ID NO: 41, SEQ ID NO: 42, SEQ ID NO: 43, or SEQ ID NO: 44.

In some embodiments, the HRAS protein is the wild-type HRAS isoform 1 protein (SEQ ID NO: 12). In some embodiments, the KRAS protein is an HRAS isoform 1 variant, for example, a variant comprising at least 70% (e.g., 70%, 75%, 80%, 85%, 90%, 95%, 96%, 97%, 98%, 99%, 100%, or ranges therebetween) sequence identity with SEQ ID NO: 12. In some embodiments, the HRAS isoform 1 variant is an active variant (e.g., constitutively active) comprising at least 70% (e.g., 70%, 75%, 80%, 85%, 90%, 95%, 96%, 97%, 98%, 99%, 100%, or ranges therebetween) sequence identity with SEQ ID NO: 12.

In some embodiments, the HRAS protein is the wild-type HRAS isoform 2 protein (SEQ ID NO: 14). In some embodiments, the HRAS protein is an HRAS isoform 2 variant, for example, a variant comprising at least 70% (e.g., 70%, 75%, 80%, 85%, 90%, 95%, 96%, 97%, 98%, 99%, 100%, or ranges therebetween) sequence identity with SEQ ID NO: 14. In some embodiments, the HRAS isoform 2 variant is an active variant (e.g., constitutively active) comprising at least 70% (e.g., 70%, 75%, 80%, 85%, 90%, 95%, 96%, 97%, 98%, 99%, 100%, or ranges therebetween) sequence identity with SEQ ID NO: 14.

In some embodiments, the HRAS protein is an HRAS variant selected from HRAS^{G12S} (SEQ ID NO: 15 or SEQ ID NO: 17), and HRAS^{G12V} (SEQ ID NO: 16 or SEQ ID NO: 18). In some embodiments, the HRAS protein is a HRAS variant with one or more substitutions (e.g., 1, 2, 3, 4, 5, 6, 7, 8, 9, 10, 15, 20, 25, 30, 40, 50, 60, or ranges therebetween) relative to SEQ ID NO: 15, SEQ ID NO: 16, SEQ ID NO: 17, or SEQ ID NO: 18.

In some embodiments, the NRAS protein is the wild-type NRAS protein (SEQ ID NO: 20). In some embodiments, the NRAS protein is an NRAS variant, for example, a variant comprising at least 70% (e.g., 70%, 75%, 80%, 85%, 90%, 95%, 96%, 97%, 98%, 99%, 100%, or ranges therebetween) sequence identity with SEQ ID NO: 20. In some embodiments, the NRAS variant is an active variant (e.g., constitutively active) comprising at least 70% (e.g., 70%, 75%, 80%, 85%, 90%, 95%, 96%, 97%, 98%, 99%, 100%, or ranges therebetween) sequence identity with SEQ ID NO: 20.

In some embodiments, the NRAS protein is an NRAS variant selected from NRAS^{G12D} (SEQ ID NO: 21) and NRAS^{Q61R} (SEQ ID NO: 22). In some embodiments, the NRAS protein is a NRAS variant with one or more substitutions (e.g., 1, 2, 3, 4, 5, 6, 7, 8, 9, 10, 15, 20, 25, 30, 40, 50, 60, or ranges therebetween) relative to SEQ ID NO: 21 or SEQ ID NO: 22.

In some embodiments, the RAS protein (e.g., KRAS, HRAS, or NRAS protein) or variant thereof is expressed/provided as a fusion and/or with a tag for detection, identification, etc. In some embodiments, the RAS protein or variant thereof is expressed/provided as a fusion with a bioluminescent reporter. In some embodiments, the RAS protein or variant thereof is expressed/provided as a fusion with a luciferase. In some embodiments, the RAS protein or variant thereof is expressed/provided as a fusion with an active variant of an Oplophorus luciferase. In some embodiments, the RAS protein or variant thereof is provided/expressed as a fusion with a bioluminescent polypeptide and/or a component of a bioluminescent complex based on (e.g., structurally, functionally, etc.) the luciferase of Oplophorus gracilirostris, the NanoLuc® luciferase (Promega Corporation, see U.S. Pat. Nos. 8,557,970 and 8,669,103, herein incorporated by reference in their entireties) (SEQ ID NO: 23), NanoBiT (Promega Corporation, see U.S. Pat. No. 9,797,889, herein incorporated by reference in its entirety), or NanoTrip (see U.S. Pat. Pub. No. 2020/0270586 and U.S. patent application Ser. No. 17/105,925, each of which is herein incorporated by reference in its entirety). In some embodiments, methods and systems herein incorporate commercially available NanoLuc®-based technologies (e.g., NanoLuc® luciferase, NanoBRET, NanoBiT, NanoTrip, NanoGlo, etc.), but in other embodiments, various combinations, variations, or derivations from the commercially available NanoLuc®-based technologies are employed.

In some embodiments, the RAS protein (e.g., KRAS, HRAS, or NRAS protein) is expressed/provided as a fusion with a bioluminescent polypeptide including but not limited to NanoLuc® and/or the bioluminescent polypeptides described in PCT Appln. No. PCT/US2010/033449, U.S. Pat. No. 8,557,970, PCT Appln. No. PCT/2011/059018, and U.S. Pat. No. 8,669,103 (each of which is herein incorporated by reference in its entirety and for all purposes). In some embodiments, such bioluminescent polypeptides are linked (e.g., fused, chemically linked, etc.) to the RAS protein for use in the methods and systems described herein.

In some embodiments, the RAS protein (e.g., KRAS, HRAS, or NRAS protein) is expressed/provided as a fusion with a component of a bioluminescent complex, including but not limited to NanoBiT®, NanoTrip, and/or the peptide and polypeptide components of bioluminescent complexes described in, for example, PCT Appln. No. PCT/US2014/026354; U.S. Pat. No. 9,797,889; U.S. Pat. Pub. No. 2020/0270586 (WO 2019/241438); and U.S. patent application Ser. No. 17/105,925 (each of which is herein incorporated by reference in its entirety and for all purposes). In some embodiments, such peptide and/or polypeptide components of bioluminescent complexes are linked (e.g., fused, chemically linked, etc.) to the RAS protein for use in the methods and systems described herein. For example, in some embodiments, the RAS protein is expressed/provided as a fusion with LgBiT (SEQ ID NO: 25), SmBiT (SEQ ID NO: 26), LgTrip 3092 (SEQ ID NO: 27), LgTrip 3546 (SEQ ID NO: 28), LgTrip 2098 (SEQ ID NO: 29), or SmTrip9 (SEQ ID NO: 30).

As disclosed in U.S. Pat. Nos. 10,024,862 and 9,977,586 (each of which is herein incorporated by reference in its entireties and for all purposes), a RAS protein that is linked (e.g., fused) to a bioluminescent reporter (e.g., luciferase, component of the bioluminescent complex, etc.) can be detected by bioluminescence resonance energy transfer (BRET) between the bioluminescent reporter and an energy acceptor (e.g., a fluorophore) present in the system or method and co-localized with the protein (e.g., kinase).

In some embodiments, any of the NanoLuc®-based, NanoBiT-based, and/or NanoTrip-based peptides, polypeptide, complexes, fusions, and conjugates may find use in BRET-based applications with the systems and methods described herein. For example, in certain embodiments, provided herein is a RAS protein (e.g., KRAS, HRAS, or NRAS protein) or variant thereof that is fused to a bioluminescent reporter (e.g., NanoLuc®-based, NanoBiT-based, and/or NanoTrip-based polypeptide, peptide, or complex), and a RAS binding agent comprising an energy acceptor (e.g., a fluorophore (e.g., fluorescent protein, small molecule fluorophore, etc.)), wherein the emission spectrum of the NanoLuc®-based, NanoBiT-based, and/or NanoTrip-based polypeptide, peptide, or complex overlaps the excitation spectrum of the energy acceptor (e.g., a fluorophore). In some embodiments, upon engagement of the RAS binding agent with the RAS protein, and in the presence of a substrate (e.g., coelenterazine, furimazine, etc.) for the bioluminescent reporter, BRET is detected. In some embodiments, upon binding of a candidate RAS binding compound to the RAS protein, the RAS binding agent is displaced, and a decrease in BRET is detected.

### III. RAS BINDING AGENTS AND COMPOSITIONS

Disclosed herein are RAS binding agents, including KRAS binding agents, HRAS binding agents, and NRAS binding agents, and systems and methods using the RAS binding agents. The RAS binding agent includes a RAS binding moiety and a functional element. In some embodiments, the RAS binding moiety and the functional element are connected by a covalent bond. In some embodiments, the RAS binding moiety and the functional element are connected by a linker.

The RAS binding moiety can be any moiety known to bind to the RAS protein (e.g., KRAS, HRAS, or NRAS protein). In some embodiments, the RAS binding moiety is a KRAS binding moiety. In some embodiments, the RAS binding moiety is an HRAS binding moiety. In some embodiments, the RAS binding moiety is an NRAS binding moiety. In some embodiments, the RAS binding moiety is a moiety that binds to the RAS SI/SII site. In some embodiments, the KRAS binding moiety is a moiety that binds to the KRAS SI/SII site. In some embodiments, the HRAS binding moiety is a moiety that binds to the HRAS SI/SII site. In some embodiments, the NRAS binding moiety is a moiety that binds to the NRAS SI/SII site.

In some embodiments, the RAS binding agent is a compound of formula (I):

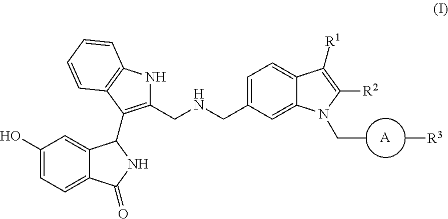

or a salt thereof, wherein:

A is a monocyclic aryl or heteroaryl;

one of R¹, R², and R³ is a group -Linker-B, wherein B is a functional element; and

the other two of R¹, R², and R³ are independently selected from hydrogen and methyl.

In some embodiments, A is a monocyclic aryl or a monocyclic heteroaryl having 1, 2, or 3 heteroatoms independently selected from N, S, and O. In some embodiments, A is a monocyclic aryl or a monocyclic heteroaryl having 1 or 2 nitrogen atoms. In some embodiments, A is selected from phenyl, imidazole, pyrrole, pyridyl, thiophene, and triazole. In some embodiments, A is selected from phenyl, imidazole, and pyrrole.

In some embodiments, A has a formula selected from:

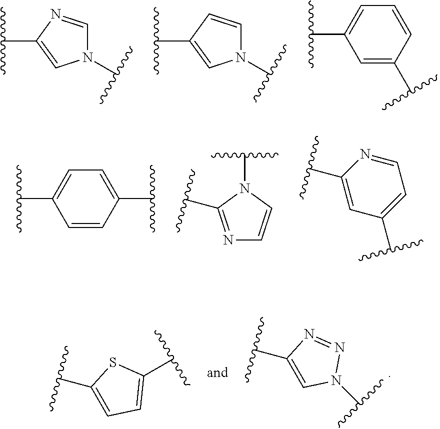

In some embodiments, A has a formula selected from:

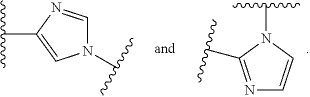 [SMILES: *c1cn(*)cn1 ; *c1nccn1*]

In some embodiments, A has formula:

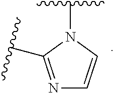 [SMILES: *c1nccn1*]

In some embodiments, R¹ is a group -Linker-B, and R² and R³ are independently selected from hydrogen and methyl. In some embodiments, R¹ is a group -Linker-B, R² is hydrogen, and R³ is hydrogen or methyl. In some embodiments, R¹ is a group -Linker-B, R² is hydrogen, and R³ is methyl.

In some embodiments, R² is a group -Linker-B, and R¹ and R³ are independently selected from hydrogen and methyl. In some embodiments, R² is a group -Linker-B, R¹ is hydrogen, and R³ is hydrogen or methyl. In some embodiments, R² is a group -Linker-B, R¹ is hydrogen, and R³ is methyl.

In some embodiments, R³ is a group -Linker-B, and R¹ and R² are independently selected from hydrogen and methyl. In some embodiments, R³ is a group -Linker-B, and R¹ and R² are both hydrogen.

In some embodiments, the compound of formula (I) has a structure selected from:

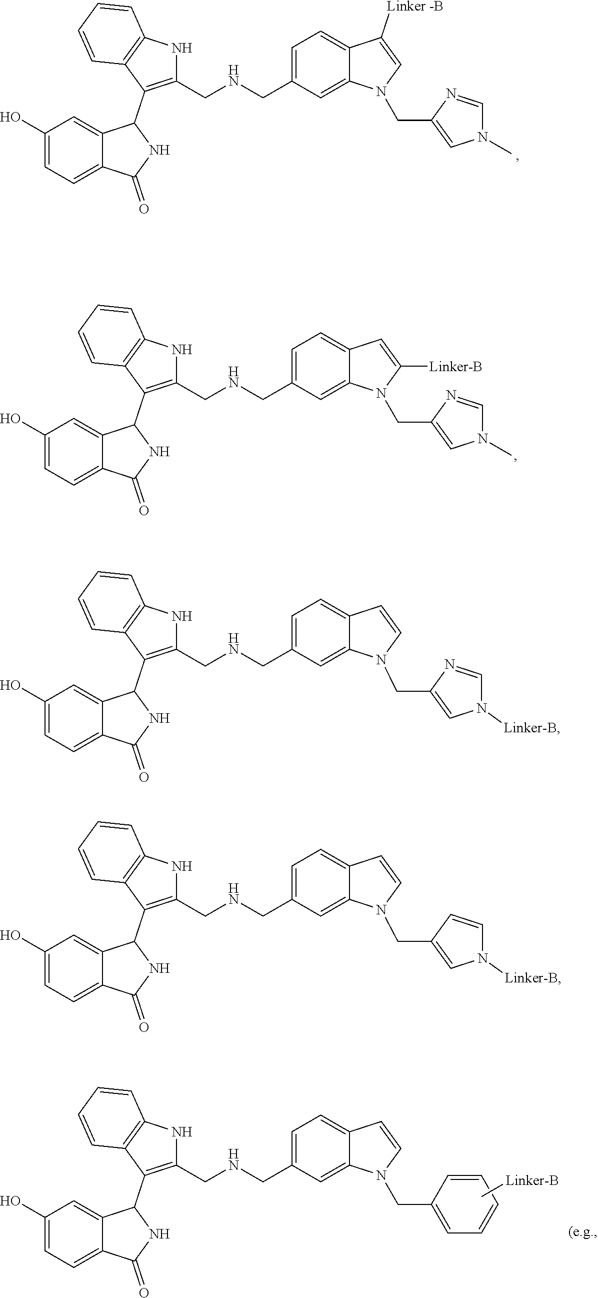

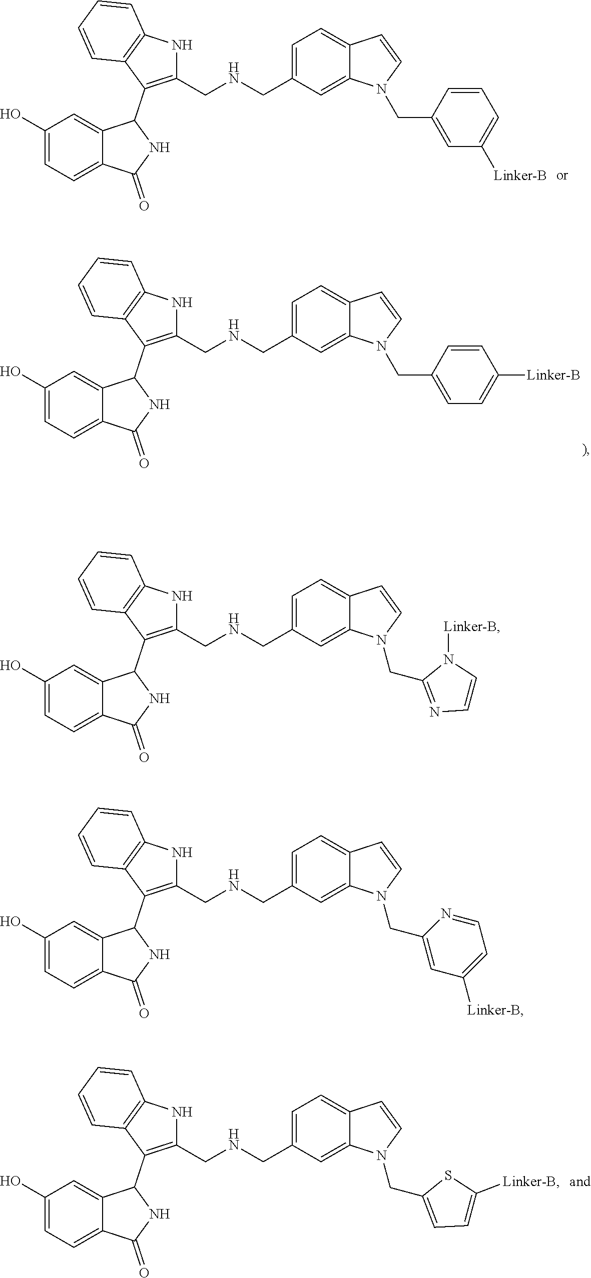

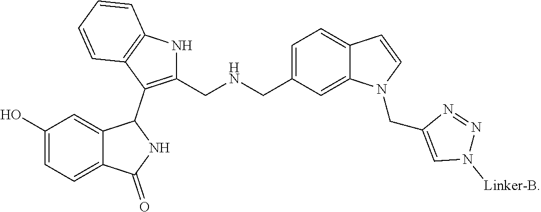

In some embodiments, the compound of formula (I) has a structure selected from:

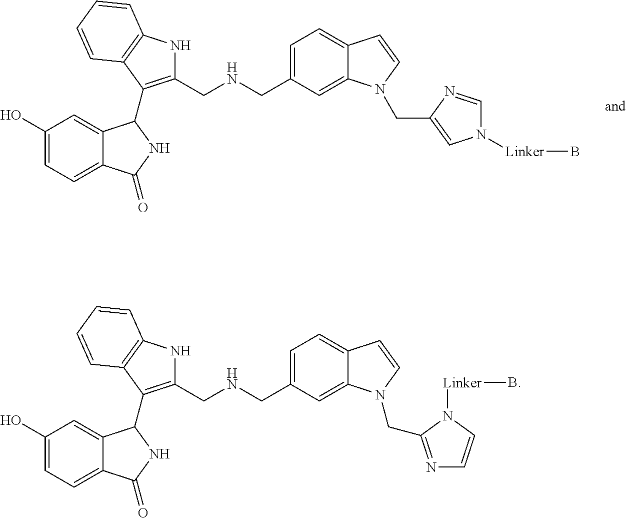

In some embodiments, the compound of formula (I) has a structure:

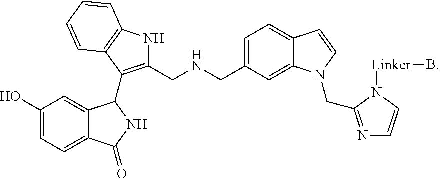

The compound of formula (I) includes a linker as part of the group -Linker-B. In some embodiments, the linker provides sufficient distance between the functional element B and the rest of the compound, to allow each to function undisturbed (or minimally disturbed) by the linkage to the other. For example, in some embodiments, such as when the group B is a detectable element (as further described herein), the linker provides sufficient distance to allow the compound of formula (I) to bind to the RAS protein (e.g., KRAS, HRAS, or NRAS) and also to allow the detectable moiety to be detectable (e.g., without interference or with minimal interference). In some embodiments, the linker separates the functional element (e.g., detectable element, solid surface, etc.) from the rest of the compound of formula (I) by 5 Å to 1000 Å, inclusive, in length. In some embodiments, the linker separates the functional element from the rest of the compound of formula (I) by 5 Å, 10 Å, 20 Å, 50 Å, 100 Å, 150 Å, 200 Å, 300 Å, 400 Å, 500 Å, 600 Å, 700 Å, 800 Å, 900 Å, 1000 Å, or any suitable range therebetween (e.g., 5-100 Å, 50-500 Å, 150-700 Å, etc.). In some embodiments, the linker separates the functional element from the rest of the compound of formula (I) by 1-200 atoms (e.g., 1, 2, 3, 4, 5, 6, 7, 8, 9, 10, 15, 20, 25, 30, 35, 40, 45, 50, 60, 70, 80, 90, 100, 120, 140, 160, 180, 200, or any suitable ranges therebetween (e.g., 2-20, 10-50, etc.)).

The linker can include one or more groups independently selected from methylene (—CH₂—), ether (—O—), amine (—NH—), alkylamine (—NR—, wherein R is an optionally substituted C₁-C₆ alkyl group), thioether (—S—), disulfide (—S—S—), amide (—C(O)NH—), ester (—C(O)O—), carbamate —OC O)NH—), sulfonamide (—S(O)₂NH—), phenylene (—C₆H₄—), and piperazinylene

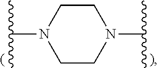 [SMILES: *N1CCN(*)CC1]

and any combination thereof.

In some embodiments, the linker comprises one or more —(CH₂CH₂O)— (oxyethylene) groups, e.g., 1-20 —(CH₂CH₂O)— groups (e.g., 1, 2, 3, 4, 5, 6, 7, 8, 9, 10, 11, 12, 13, 14, 15, 16, 17, 18, 19, or 20 —(CH₂CH₂O)— groups, or any range therebetween). In some embodiments, the linker comprises a —(CH₂CH₂O)—, —(CH₂CH₂O)₂—, —(CH₂CH₂O)₃—, —(CH₂CH₂O)₄—, —(CH₂CH₂O)₅—, —(CH₂CH₂O)₆—, —(CH₂CH₂O)₇—, —(CH₂CH₂O)₈—, —(CH₂CH₂O)₉—, or —(CH₂CH₂O)₁₀— group. In some embodiments, the linker comprises a —(CH₂CH₂O)₄— group.

In some embodiments, the linker comprises one or more alkylene groups (e.g., —(CH₂)—, wherein n is 1-12, e.g., 1, 2, 3, 4, 5, 6, 7, 8, 9, 10, 11, or 12, or any suitable range therebetween). In some embodiments, the linker comprises one or more branched alkylene groups.

In some embodiments, the linker comprises at least one amide group (—C(O)NH—). In some embodiments, the linker comprises two amide groups.

In some embodiments, the linker comprises at least one piperazinylene group.

In some embodiments, the linker comprises a cleavable (e.g., enzymatically cleavable, chemically cleavable, etc.) moiety.

In some embodiments, the linker comprises one or more substituents, pendants, side chains, etc., comprising any suitable organic functional groups (e.g., —OH, —NH₂, —SH, —CN, ═O, ═S, halogen (e.g., —F, —Cl, —Br, —I), —COOH, —CONH₂, —CH₃, etc.).

In some embodiments, the linker comprises more than one linearly connected C, S, N, and/or O atoms. In some embodiments, the linker comprises 1-200 linearly connected atoms (e.g., 1, 2, 3, 4, 5, 6, 7, 8, 9, 10, 15, 20, 25, 30, 35, 40, 45, 50, 60, 70, 80, 90, 100, 120, 140, 160, 180, 200, or any suitable ranges therebetween (e.g., 2-20, 10-50, 6-18)). In some embodiments, the linker comprises 1-200 linearly connected atoms (e.g., 1, 2, 3, 4, 5, 6, 7, 8, 9, 10, 15, 20, 25, 30, 35, 40, 45, 50, 60, 70, 80, 90, 100, 120, 140, 160, 180, 200, or any suitable ranges therein (e.g., 2-20, 10-50, 6-18)).

In some embodiments, the linker has formula:

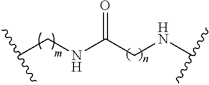

wherein m and n are independently 0, 1, 2, 3, 4, 5, or 6. In some embodiments, m and n are independently 0, 1, 2, or 3. In some embodiments, m and n are independently 1, 2, or 3.

In some embodiments, the linker has formula:

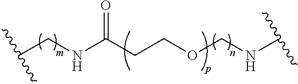

wherein m, n, and p are independently 0, 1, 2, 3, 4, 5, or 6. In some embodiments, m, n, and p are independently 0, 1, 2, 3, or 4. In some embodiments, m and n are independently 1, 2, or 3, and p is 1, 2, 3, 4, 5, or 6.

In some embodiments, the linker has formula:

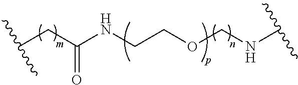

wherein m, n, and p are independently 0, 1, 2, 3, 4, 5, or 6. In some embodiments, m, n, and p are independently 0, 1, 2, 3, or 4. In some embodiments, m and n are independently 1, 2, 3, or 4, and p is 1, 2, 3, 4, 5, or 6.

In some embodiments, the linker has formula:

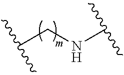

wherein m is 0, 1, 2, 3, 4, 5, or 6. In some embodiments, m is 1, 2, or 3. In some embodiments, m is 1 or 2.

In some embodiments, the linker has formula:

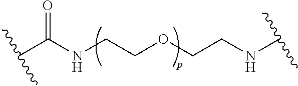

wherein p is 1, 2, 3, 4, 5, or 6.

In some embodiments, the linker is selected from:

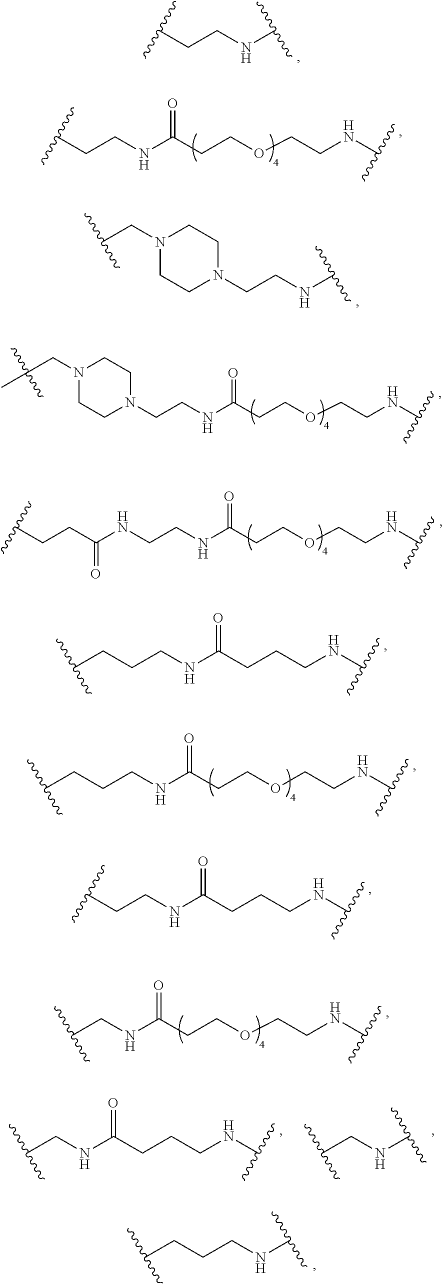

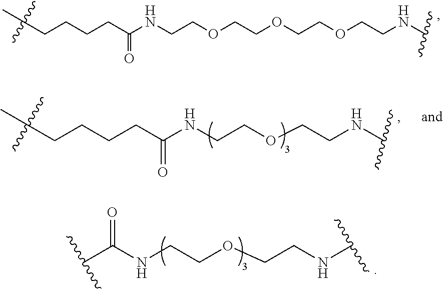

In some embodiments, Linker is selected from:

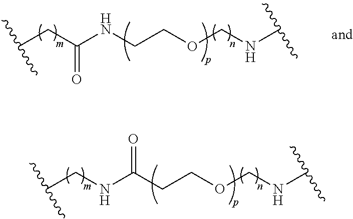

wherein m, n, and p are independently 0, 1, 2, 3, 4, 3, or 6.

The compound of formula (I) includes a functional element as part of the group -Linker-B (where B is the functional element). In certain embodiments, the functional element has a detectable property that allows for detection of the RAS binding agent. Detectable elements include those with a characteristic electromagnetic spectral property such as emission or absorbance, magnetism, electron spin resonance, electrical capacitance, dielectric constant, or electrical conductivity as well as functional groups which are ferromagnetic, paramagnetic, diamagnetic, luminescent, electrochemiluminescent, fluorescent, phosphorescent, chromatic, antigenic, or have a distinctive mass. A detectable element includes, but is not limited to, a nucleic acid molecule (e.g., DNA or RNA (e.g., an oligonucleotide or nucleotide), a protein (e.g., a luminescent protein), a peptide, a radionuclide, an affinity tag (e.g., biotin or streptavidin), a hapten, an amino acid, a lipid, a lipid bilayer, a solid support, a fluorophore, a chromophore, a reporter molecule, an electron opaque molecule, a MRI contrast agent (e.g., manganese, gadolinium(III), or iron-oxide particles) or a coordinator thereof, a SPECT contrast agent, or the like. Methods to detect a particular detectable element, or to isolate a composition comprising a particular detectable element and anything bound thereto, are understood, and include methods such as fluorescence, mass spectrometry, radionuclide detection, optical imaging, magnetic resonance imaging (MRI), single photon emission computed tomography (SPECT), and energy transfer.

In some embodiments, the functional element is or comprises a solid support. Suitable solid supports include a sedimental particle such as a magnetic particle, a sepharose, or cellulose bead; a membrane; glass, e.g., glass slides; cellulose, alginate, plastic, or other synthetically prepared polymer (e.g., an Eppendorf tube or a well of a multi-well plate); self-assembled monolayers; a surface plasmon resonance chip; a solid support with an electron conducting surface; etc.

Exemplary functional elements include haptens (e.g., molecules useful to enhance immunogenicity such as keyhole limpet hemacyanin), cleavable labels (e.g., photocleavable biotin) and fluorescent labels (e.g., N-hydroxysuccinimide (NHS) modified coumarin and succinimide or sulfonosuccinimide modified BODIPY (which can be detected by UV and/or visible excited fluorescence detection), rhodamine (R110, rhodols, CRG6, Texas Methyl Red (TAMRA), Rox5, FAM, or fluorescein), coumarin derivatives (e.g., 7 aminocoumarin and 7-hydroxycoumarin), 2-amino-4-methoxynapthalene, 1-hydroxypyrene, resorufin, phenalenones or benzphenalenones (U.S. Pat. No. 4,812,409)), acridinones (U.S. Pat. No. 4,810,636), anthracenes, and derivatives of alpha and beta-naphthol, fluorinated xanthene derivatives including fluorinated fluoresceins and rhodols (e.g., U.S. Pat. No. 6,162,931), and bioluminescent molecules (e.g., luciferase (e.g., Oplophorus-derived luciferase (see, e.g., U.S. Pat. Pub. No. 2010/0281552 and U.S. Pat. Pub. No. 2012/0174242, herein incorporated by reference in their entireties) or GFP or GFP derivatives).

Another class of detectable elements includes molecules detectable using electromagnetic radiation and includes, but is not limited to, xanthene fluorophores, dansyl fluorophores, coumarins and coumarin derivatives, fluorescent acridinium moieties, benzopyrene-based fluorophores as well as 7-nitrobenz-2-oxa-1,3-diazole, and 3-N-(7-nitrobenz-2-oxa-1,3-diazol-4-yl)-2,3-diamino-propionic acid. Preferably, the fluorescent molecule has a high quantum yield of fluorescence at a wavelength different from native amino acids and more preferably has high quantum yield of fluorescence that can be excited in the visible, or in both the UV and visible, portion of the spectrum. Upon excitation at a preselected wavelength, the molecule is detectable at low concentrations either visually or using conventional fluorescence detection methods. Electrochemiluminescent molecules such as ruthenium chelates and its derivatives or nitroxide amino acids and their derivatives are detectable at femtomolar ranges and below.

In some embodiments, the detectable element is an energy acceptor, such as a fluorophore. Suitable fluorophores include, but are not limited to: xanthene derivatives (e.g., fluorescein, rhodamine, Oregon green, eosin, Texas red, etc.), cyanine derivatives (e.g., cyanine, indocarbocyanine, oxacarbocyanine, thiacarbocyanine, merocyanine, etc.), naphthalene derivatives (e.g., dansyl and prodan derivatives), oxadiazole derivatives (e.g., pyridyloxazole, nitrobenzoxadiazole, benzoxadiazole, etc.), pyrene derivatives (e.g., cascade blue), oxazine derivatives (e.g., Nile red, Nile blue, cresyl violet, oxazine 170, etc.), acridine derivatives (e.g., proflavin, acridine orange, acridine yellow, etc.), arylmethine derivatives (e.g., auramine, crystal violet, malachite green, etc.), tetrapyrrole derivatives (e.g., porphin, phthalocyanine, bilirubin, etc.), CF dye (Biotium), BODIPY (Invitrogen), ALEXA FLuoR (Invitrogen), DYLIGHT FLUOR (Thermo Scientific, Pierce), ATTO and TRACY (Sigma Aldrich), FluoProbes (Interchim), DY and MEGASTOKES (Dyomics), SULFO CY dyes (CYANDYE, LLC), SETAU AND SQUARE DYES (SETA BioMedicals), QUASAR and CAL FLUOR dyes (Biosearch Technologies), SURELIGHT DYES (APC, RPE, PerCP, Phycobilisomes)(Columbia Biosciences), APC, APCXL, RPE, BPE (Phyco-Biotech), autofluorescent proteins (e.g., YFP, RFP, mCherry, mKate), quantum dot nanocrystals, etc. In some embodiments, the fluorophore is a rhodamine analog (e.g., carboxy rhodamine analog) such as those described in U.S. Pat. Pub. No. 2013/0317207, herein incorporated by reference in its entirety. In some embodiments, the fluorophore is a BODIPY dye. In some embodiments, the fluorophore is DY-605 (Dyomics).

In some embodiments, the detectable element is an energy acceptor (fluorophore) having formula:

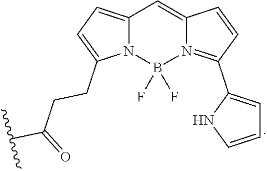

In some embodiments, the detectable element is an energy acceptor (fluorophore) having formula:

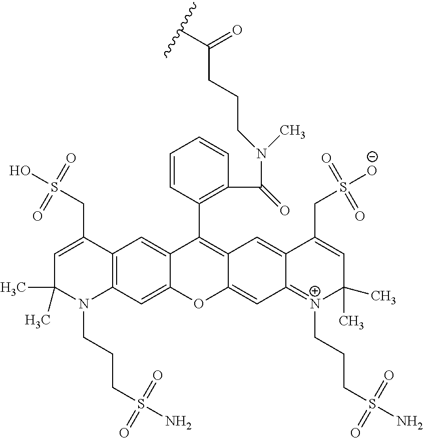 [SMILES: *C(=O)CCCN(C)C(=O)c1ccccc1C1=c2cc3c(cc2Oc2cc4c(cc21)C(CS(=O)(=O)O)=CC(C)(C)N4CCCS(N)(=O)=O)=[N+](CCCS(N)(=O)=O)C(C)(C)C=C3CS(=O)(=O)[O-]]

or a salt or a tautomer thereof.

In addition to fluorescent molecules, a variety of molecules with physical properties based on the interaction and response of the molecule to electromagnetic fields and radiation find use as the detectable moiety in the RAS binding agents disclosed herein. These properties include absorption in the UV, visible, and infrared regions of the electromagnetic spectrum, presence of chromophores that are Raman active and can be further enhanced by resonance Raman spectroscopy, electron spin resonance activity, and nuclear magnetic resonances and molecular mass, e.g., via a mass spectrometer.

In some embodiments, a functional element is a capture element. In some embodiments, a capture element is a substrate for a protein (e.g., enzyme), and the capture agent is that protein. In some embodiments, a capture element is a “covalent substrate” or one that forms a covalent bond with a protein or enzyme that it reacts with. The substrate may comprise a reactive group (e.g., a modified substrate) that forms a covalent bond with the enzyme upon interaction with the enzyme, or the enzyme may be a mutant version that is unable to reconcile a covalently bound intermediate with the substrate. In some embodiments, the substrate is recognized by a mutant protein (e.g., mutant dehalogenase), which forms a covalent bond thereto. In such embodiments, while the interaction of the substrate and a wild-type version of the protein (e.g., dehalogenase) results in a product and the regeneration of the wild-type protein, interaction of the substrate (e.g., haloalkane) with the mutant version of the protein (e.g., dehalogenase) results in stable bond formation (e.g., covalent bond formation) between the protein and substrate. The substrate may be any suitable substrate for any mutant protein that has been altered to form an ultra-stable or covalent bond with its substrate that would ordinarily only transiently bound by the protein. In some embodiments, the protein is a mutant hydrolase or dehalogenase. In some embodiments, the protein is a mutant dehalogenase, and the substrate is a haloalkane. In some embodiments, the haloalkane comprises an alkane (e.g., C₂-C₂₀) capped by a terminal halogen (e.g., Cl, Br, F, I, etc.). In some embodiments, the haloalkane is of the formula A-X, wherein X is a halogen (e.g., Cl, Br, F, I, etc.), and wherein A is an alkane comprising 2-20 carbons. In certain embodiments, A comprises a straight-chain segment of 2-12 carbons. In certain embodiments, A is a straight-chain segment of 2-12 carbons. In some embodiments, the haloalkane may comprise any additional pendants or substitutions that do not interfere with interaction with the mutant dehalogenase.

In some embodiments, a capture agent is a SNAP-Tag and a capture element is benzyl guanine (See, e.g., Crivat G, Taraska J W (January 2012) Trends in Biotechnology 30 (1): 8-16, herein incorporated by reference in its entirety). In some embodiments, a capture agent is a CLIP-Tag and a capture element is benzyl cytosine (See, e.g., Gautier, et al. Chem Biol. 2008, 15(2):128-36, herein incorporated by reference in its entirety).

Systems comprising mutant proteins (e.g., mutant hydrolases (e.g., mutant dehalogenases) that covalently bind their substrates (e.g., haloalkane substrates) are described, for example, in U.S. Pat. Nos. 7,238,842; 7,425,436; 7,429,472; 7,867,726; each of which is herein incorporated by reference in its entirety.

In some embodiments, the functional element is an affinity element (e.g., that binds to an affinity agent). Examples of such pairs would include: an antibody as the affinity agent and an antigen as the affinity element; a His-tag as the affinity element and a nickel column as the affinity agent; a protein and small molecule with high affinity as the affinity agent and affinity element, respectively (e.g., streptavidin and biotin), etc. Examples of affinity molecules include molecules such as immunogenic molecules (e.g., epitopes of proteins, peptides, carbohydrates, or lipids (e.g., any molecule which is useful to prepare antibodies specific for that molecule)); biotin, avidin, streptavidin, and derivatives thereof; metal binding molecules; and fragments and combinations of these molecules. Exemplary affinity molecules include 5×His (HHHHH) (SEQ ID NO: 31), 6×His (HHHHHH) (SEQ ID NO: 32), C-myc (EQKLISEEDL) (SEQ ID NO: 33), FLAG (DYKDDDDK) (SEQ ID NO: 34), Strep-Tag (WSHPQFEK) (SEQ ID NO: 35), HA Tag (YPYDVPDYA) (SEQ ID NO: 36), thioredoxin, cellulose binding domain, chitin binding domain, S-peptide, T7 peptide, calmodulin binding peptide, C-end RNA tag, metal binding domains, metal binding reactive groups, amino acid reactive groups, inteins, biotin, streptavidin, and maltose binding protein. Another example of an affinity molecule is dansyllysine. Antibodies that interact with the dansyl ring are commercially available (Sigma Chemical; St. Louis, Mo.) or can be prepared using known protocols such as described in Antibodies: A Laboratory Manual (Harlow and Lane, 1988).

In some embodiments, the functional element is a moiety that induces protein degradation. For example, the functional element may be a moiety which recruits protein degradation pathways within live cells. Suitable functional elements to induce protein degradation include those disclosed in Lai et al., Nature Reviews Drug Discovery, 2017, 16, 101-114, which is incorporated herein by references in its entirety. In some embodiments, the functional element is a hydrophobic group, such as adamantane or Arg-Boc₃, which induces protein degradation through hydrophobic tagging (HyT). In some embodiments, Z is a moiety from nutlin-3a, bestatin, VHL ligand, pomalidomide, and other small molecules as disclosed in Lai et al., which induce protein degradation through proteolysis-targeting chimera (PROTAC) tagging.

In some embodiments, the RAS binding agent is biocompatible (e.g., cell compatible) and/or cell permeable. Therefore, in some embodiments, suitable functional elements (e.g., detectable elements, affinity elements, solid supports, capture elements) are ones that are cell compatible and/or cell permeable within the context of such compounds. In some embodiments, the RAS binding agent is capable of crossing the cell membrane to enter a cell (e.g., via diffusion, endocytosis, active transport, passive transport, etc.). In some embodiments, suitable functional elements and linkers are selected based on cell compatibility and/or cell permeability, in addition to their particular function.

In some embodiments, the RAS binding agent is a compound selected from:

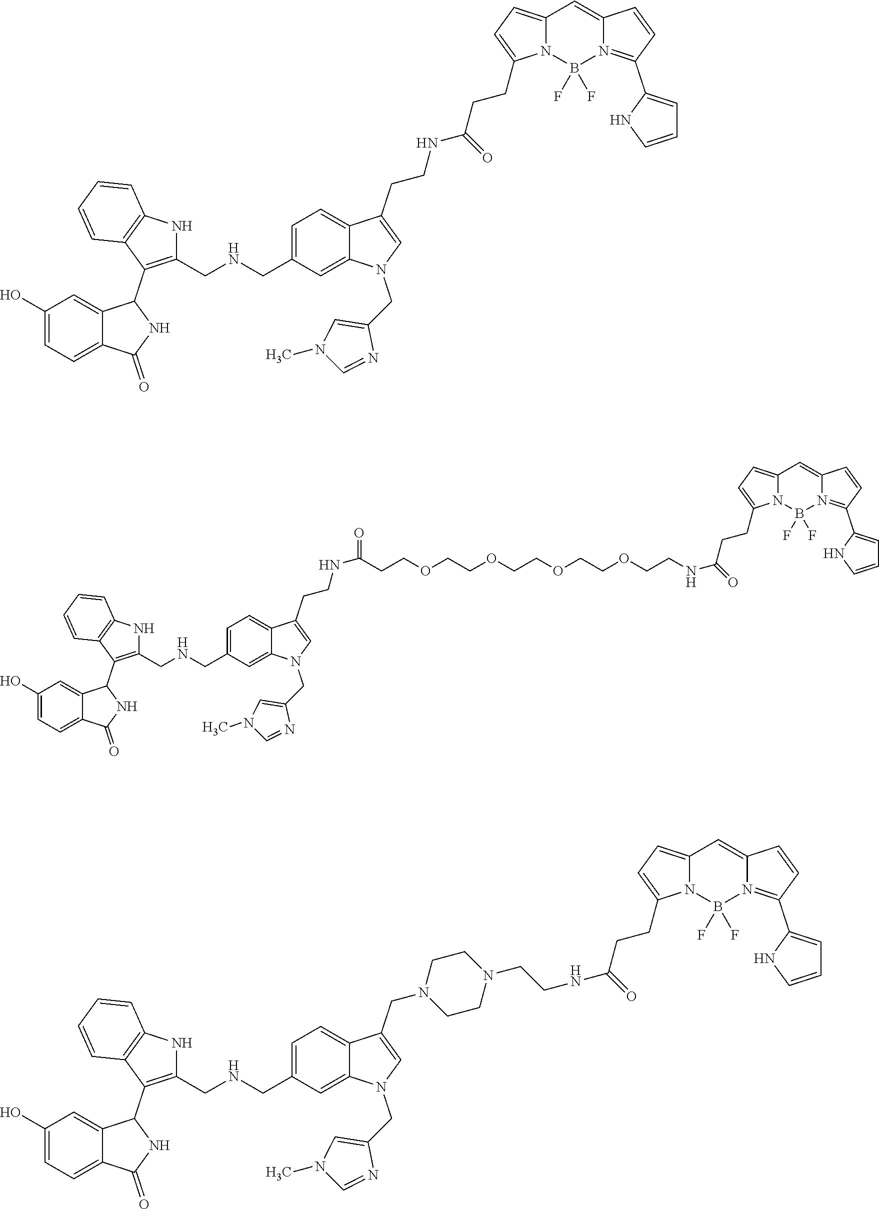

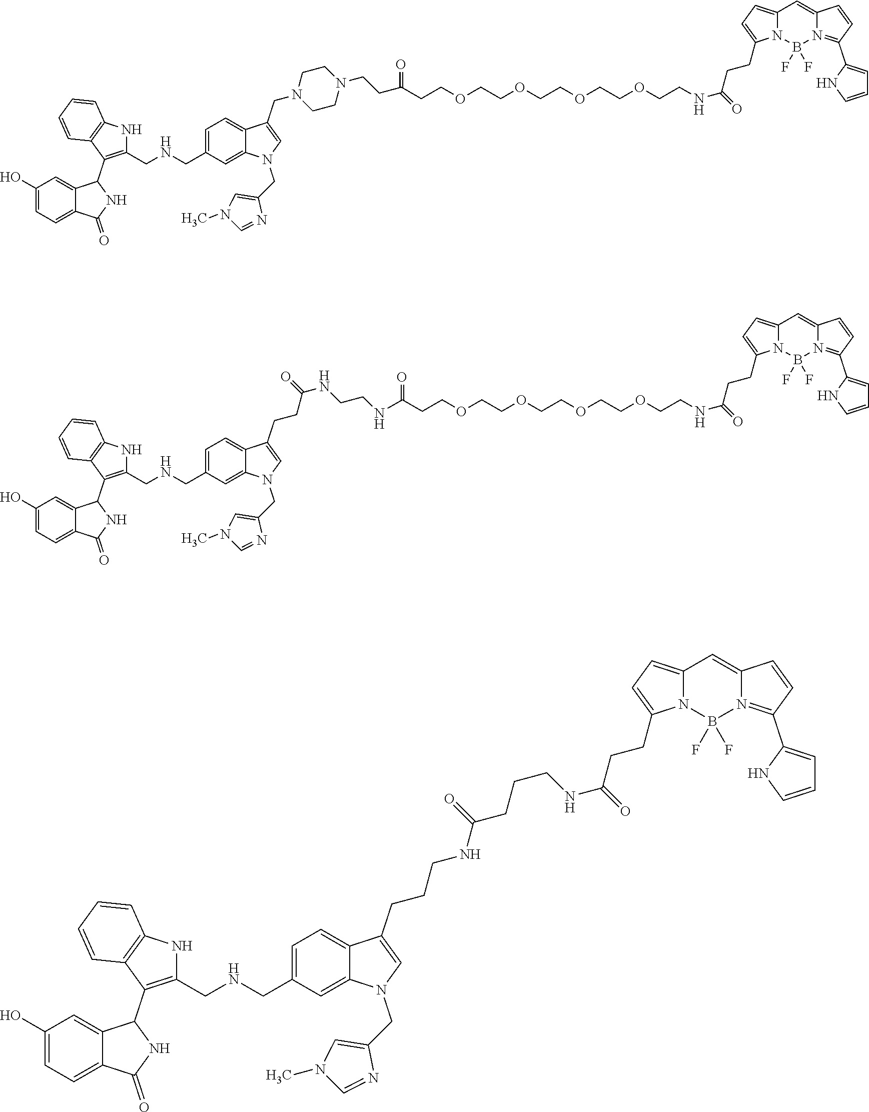

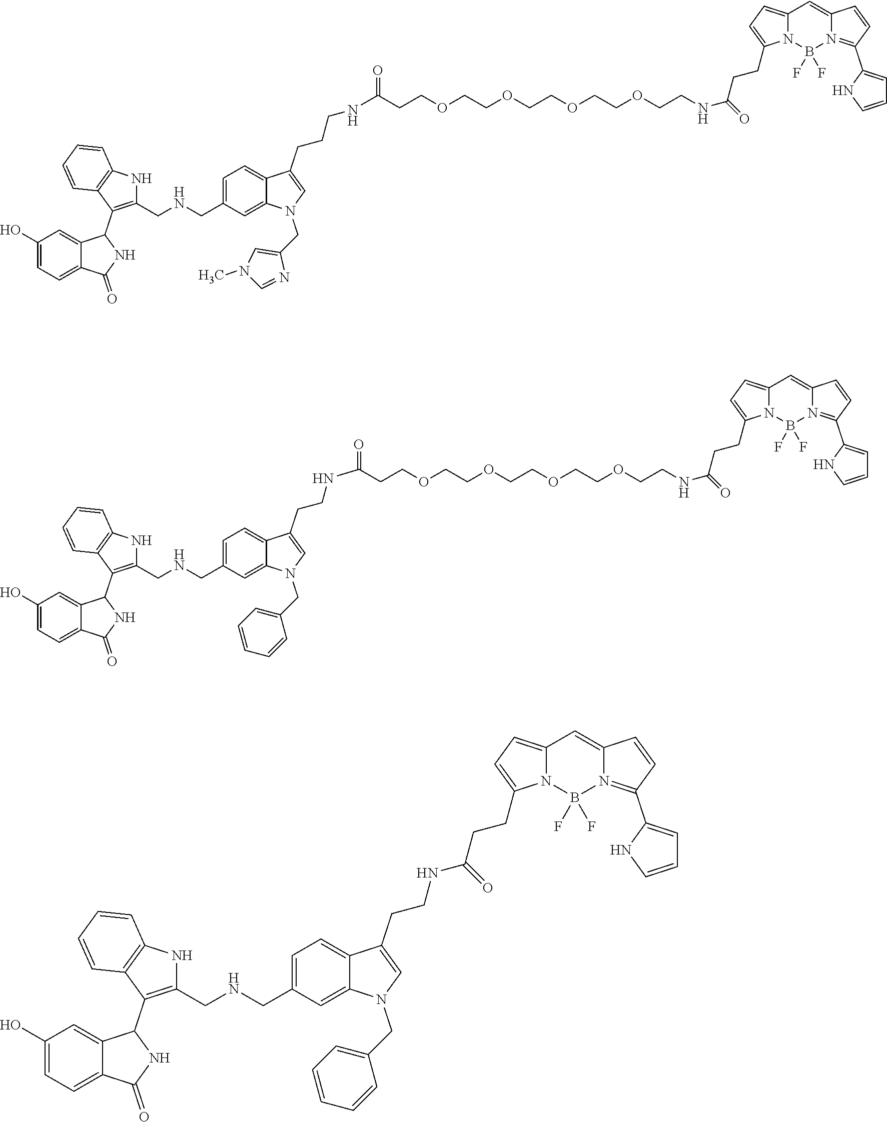

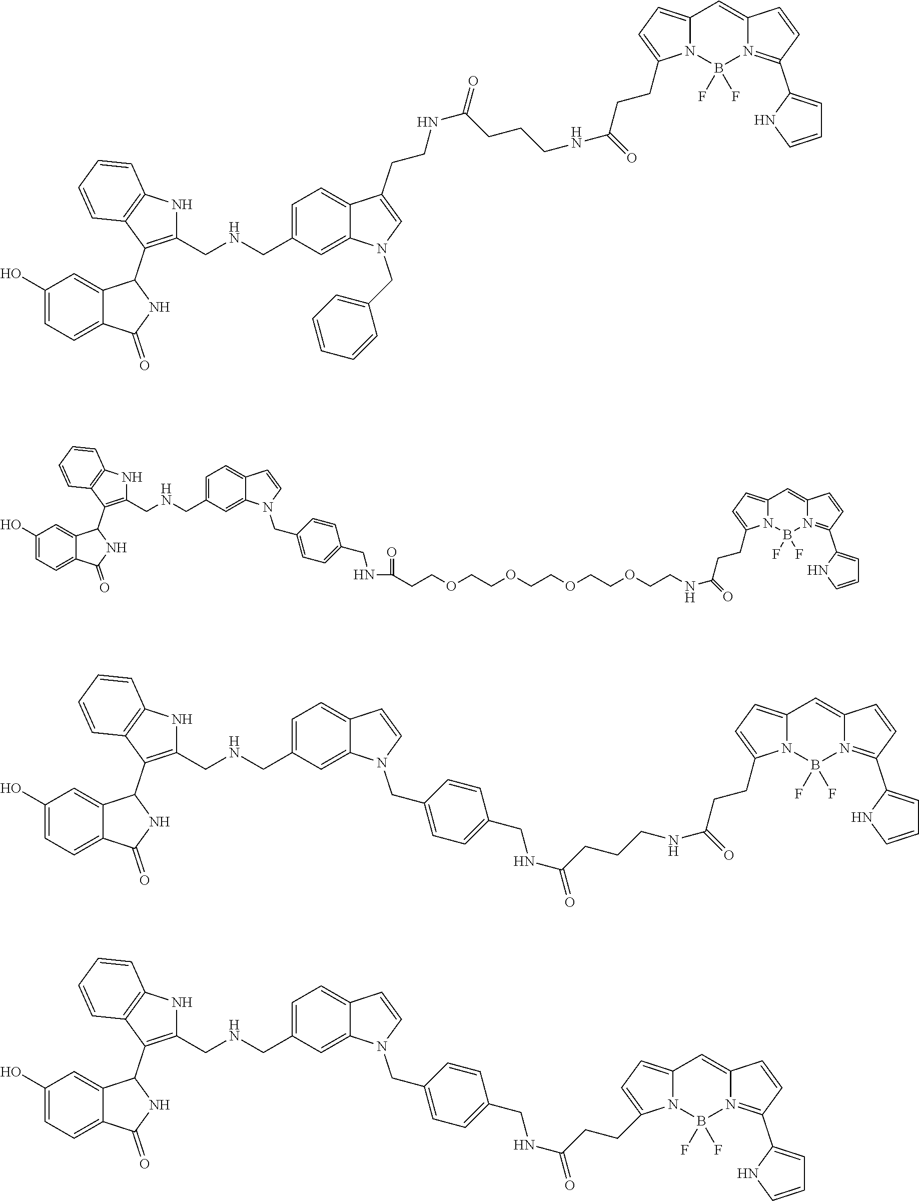

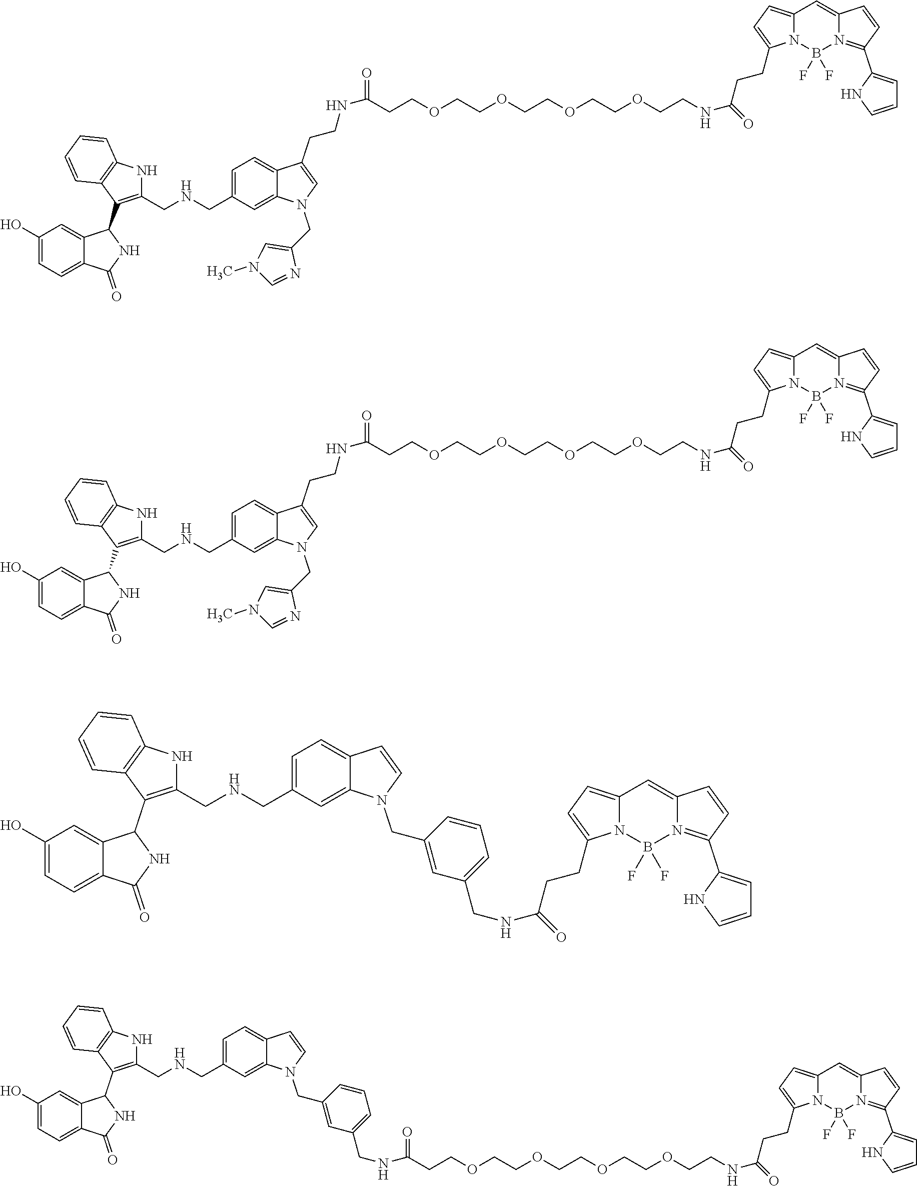

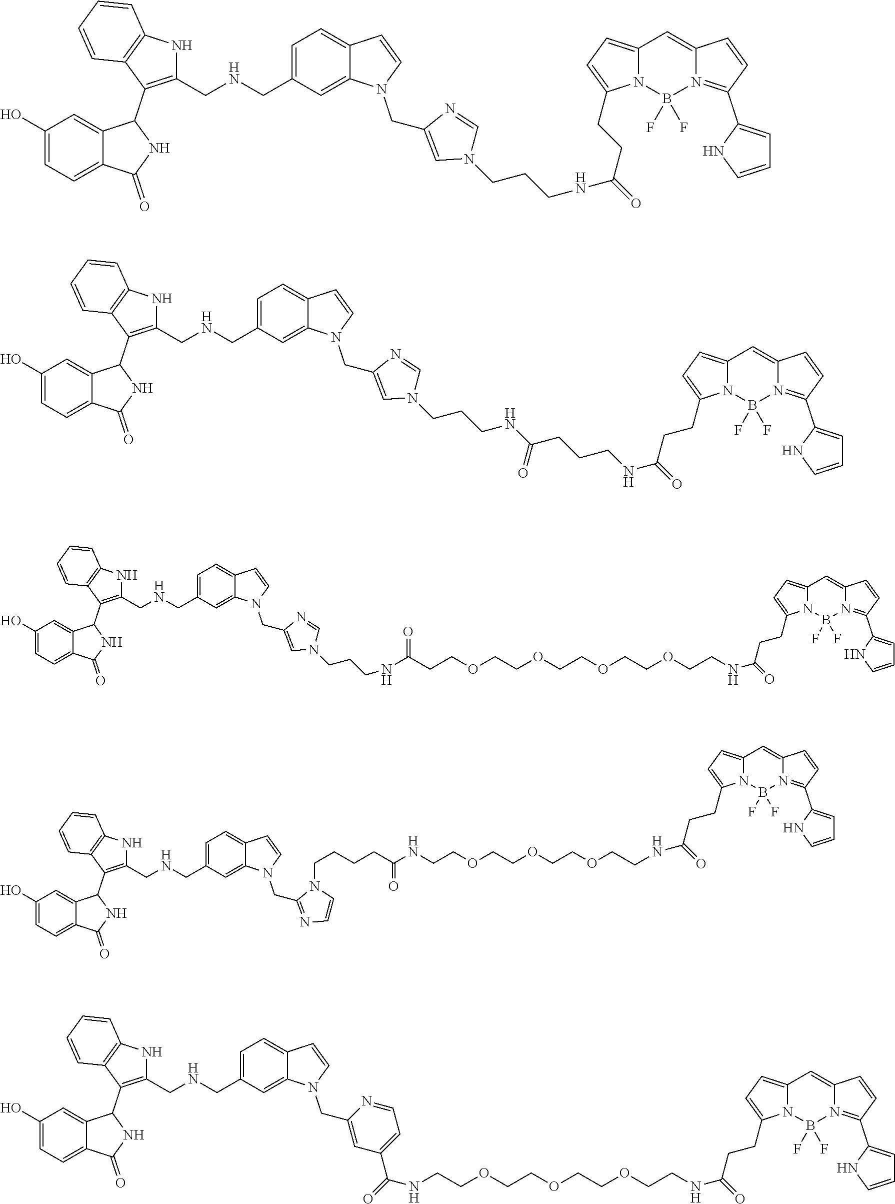

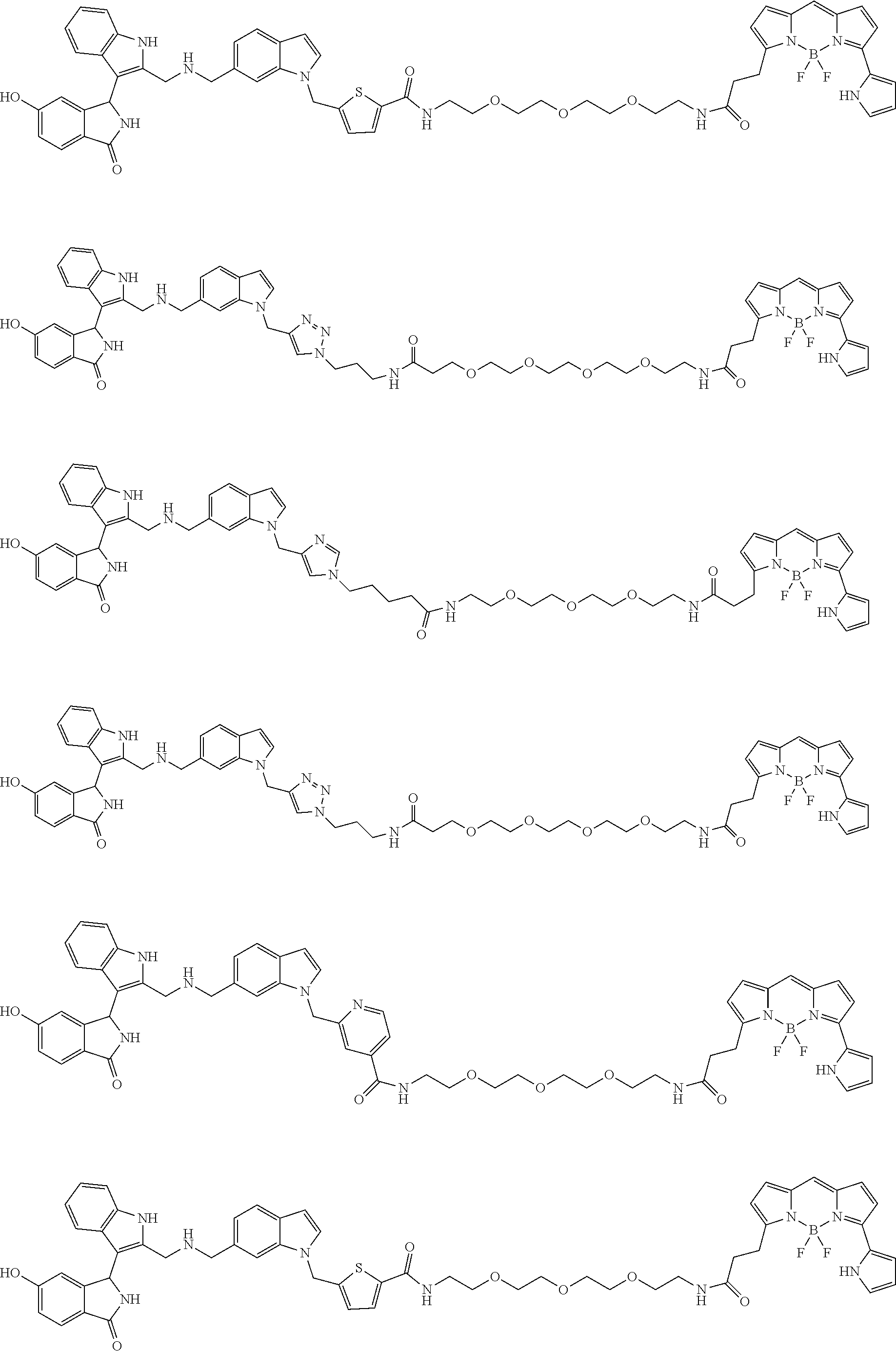

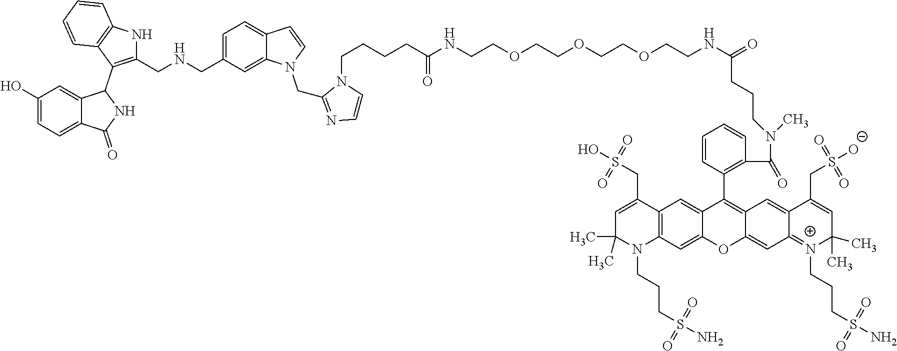 [SMILES: CN(CCCC(=O)NCCOCCOCCOCCNC(=O)CCCCn1ccnc1Cn1ccc2ccc(CNCc3[nH]c4ccccc4c3C3NC(=O)c4ccc(O)cc43)cc21)C(=O)c1ccccc1C1=c2cc3c(cc2Oc2cc4c(cc21)C(CS(=O)(=O)O)=CC(C)(C)N4CCCS(N)(=O)=O)=[N+](CCCS(N)(=O)=O)C(C)(C)C=C3CS(=O)(=O)[O-]]

or a tautomer or a salt thereof.

The RAS binding agent, such as a compound of formula (I), can be in the form of a salt. A neutral form may be regenerated by contacting the salt with a base or acid and isolating the parent compound in a conventional manner. The parent form of the compound differs from the various salt forms in certain physical properties, such as solubility in polar solvents, but otherwise the salts are equivalent to the parent form of the compound for the purposes of this disclosure.

In particular, if the RAS binding agent (e.g., compound of formula (I)) is anionic or has a functional group that may be anionic (e.g., —COOH may be —COO⁻), then a salt may be formed with one or more suitable cations. Examples of suitable inorganic cations include, but are not limited to, alkali metal cations such as Li⁺, Na⁺, and K⁺, alkaline earth cations such as Ca²⁺ and Mg²⁺, and other cations. Sodium salts may be particularly suitable. Examples of suitable organic cations include, but are not limited to, ammonium ion (i.e., NH₄⁺) and substituted ammonium ions (e.g., NH₃R₁⁺, NH₂R₂⁺, NHR₃⁺, and NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids such as lysine and arginine. In some embodiments, the compound is a sodium salt.

If the RAS binding agent (e.g., compound of formula (I)) is cationic or has a functional group that may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, tetrafluoroboric, toluenesulfonic, trifluoromethanesulfonic, and valeric. In some embodiments, the compound is a halide salt, such as a chloro, bromo, or iodo salt. In some embodiments, the compound is a tetrafluoroborate or trifluoromethanesulfonate salt.

The RAS binding agents (e.g., compounds of formula (I)) can be prepared by a variety of methods, including those shown in the Examples. The compounds and intermediates herein may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration as described for instance in “Vogel's Textbook of Practical Organic Chemistry,” 5th edition (1989), by Furniss, Hannaford, Smith, and Tatchell, pub. Longman Scientific & Technical, Essex CM20 2JE, England.

Reaction conditions and reaction times for each individual step can vary depending on the particular reactants employed and substituents present in the reactants used. Specific procedures are provided in the Examples section. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups, and the methods for protecting and deprotecting different substituents using such suitable protecting groups, are well known to those skilled in the art; examples of which can be found in the treatise by PGM Wuts entitled “Greene's Protective Groups in Organic Synthesis” (5th ed.), John Wiley & Sons, Inc. (2014), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step) or by resolution of a mixture of the stereoisomers of the compound or intermediates using a standard procedure (such as chromatographic separation, recrystallization or enzymatic resolution). Similarly, when a pure geometric isomer of a compound is required, it can be obtained by carrying out one of the above procedures using a pure geometric isomer as a starting material or by resolution of a mixture of the geometric isomers of the compound or intermediates using a standard procedure such as chromatographic separation.

The synthetic schemes and specific examples as described are illustrative and are not to be read as limiting the scope of the invention as it is defined in the claims. All alternatives, modifications, and equivalents of the synthetic methods and specific examples are included within the scope of the claims.

Also provided herein are compositions comprising the RAS binding agents. The composition may further comprise a RAS protein, such as a RAS protein described herein (e.g., a KRAS protein, an HRAS protein, an NRAS protein, or a variant of any thereof). In some embodiments, such as those in which the RAS protein is a fusion with a bioluminescent reporter, the composition further comprises a substrate for the bioluminescent reporter (e.g., coelenterazine, a coelenterazine derivative, or furimazine). In some embodiments, the composition further comprises a candidate RAS binding compound (e.g., a KRAS binding compound, an HRAS binding compound, an NRAS binding compound) such as a RAS inhibitor (e.g., a KRAS inhibitor, an HRAS inhibitor, or an NRAS inhibitor).

### IV. SYSTEMS AND METHODS

In some embodiments, provided herein are systems and methods to identify a RAS binding compound (i.e. to assess target engagement with a RAS protein by a candidate RAS binding compound). The systems and methods use the above-described RAS proteins and RAS binding agents, to identify a RAS binding compound (e.g., a RAS inhibitor).

In one aspect, provided herein is a method of identifying a RAS binding compound, the method comprising:

(a) providing a sample comprising a RAS protein; and

(b) contacting the sample with a RAS binding agent comprising a RAS binding moiety and a functional element, and a candidate RAS binding compound.

In some embodiments, the method is a method of identifying a KRAS binding compound, the method comprising:

(a) providing a sample comprising a KRAS protein; and

(b) contacting the sample with a KRAS binding agent comprising a KRAS binding moiety and a functional element, and a candidate KRAS binding compound.

In some embodiments, the method is a method of identifying an HRAS binding compound, the method comprising:

(a) providing a sample comprising an HRAS protein; and

(b) contacting the sample with an HRAS binding agent comprising an HRAS binding moiety and a functional element, and a candidate HRAS binding compound.

In some embodiments, the method is a method of identifying an NRAS binding compound, the method comprising:

(a) providing a sample comprising an NRAS protein; and

(b) contacting the sample with an NRAS binding agent comprising an NRAS binding moiety and a functional element, and a candidate NRAS binding compound.

In some embodiments, the method further comprises a step of: (c) detecting or quantifying the functional element. Methods that can be used to detect or quantify the functional element will depend on the functional element that is present in the RAS binding agent (e.g., the KRAS binding agent, HRAS binding agent, or NRAS binding agent). For example, in some embodiments, the functional element is a detectable element selected from a fluorophore, chromophore, radionuclide, electron opaque molecule, MRI contrast agent, SPECT contrast agent, and a mass tag. Accordingly, in some embodiments, the detectable element or the signal produced thereby is detected or quantified by fluorescence, optical imaging, radionuclide detection, mass spectrometry, magnetic resonance imaging (MRI), single photon emission computed tomography (SPECT), or energy transfer.

In another aspect, provided herein is a system comprising:

(a) a target RAS protein;

(b) a RAS binding agent comprising a RAS binding moiety and a functional element; and

(c) a candidate RAS binding compound.

In some embodiments, the system comprises:

(a) a target KRAS protein;

(b) a KRAS binding agent comprising a KRAS binding moiety and a functional element; and

(c) a candidate KRAS binding compound.

In some embodiments, the system comprises:

(a) a target HRAS protein;

(b) an HRAS binding agent comprising an HRAS binding moiety and a functional element; and

(c) a candidate HRAS binding compound.

In some embodiments, the system comprises:

(a) a target NRAS protein;

(b) an NRAS binding agent comprising an NRAS binding moiety and a functional element; and

(c) a candidate NRAS binding compound.

In another aspect, provided herein is a method for screening for a RAS binding compound, the method comprising:

(a) contacting a sample comprising: (i) a RAS protein; and (ii) a RAS binding agent comprising a RAS binding moiety and a functional element, with a candidate RAS binding compound; and

(b) detecting or quantifying a signal from the functional element. In some embodiments, the RAS binding compound binds the RAS protein and detectably alters the signal from the functional element.

In some embodiments, the method is a method for screening for a KRAS binding compound, the method comprising:

(a) contacting a sample comprising: (i) a KRAS protein; and (ii) a KRAS binding agent comprising a KRAS binding moiety and a functional element, with a candidate KRAS binding compound; and

(b) detecting or quantifying a signal from the functional element. In some embodiments, the KRAS binding compound binds the KRAS protein and detectably alters the signal from the functional element.

In some embodiments, the method is a method for screening for an HRAS binding compound, the method comprising:

(a) contacting a sample comprising: (i) an HRAS protein; and (ii) an HRAS binding agent comprising an HRAS binding moiety and a functional element, with a candidate HRAS binding compound; and

(b) detecting or quantifying a signal from the functional element. In some embodiments, the HRAS binding compound binds the HRAS protein and detectably alters the signal from the functional element.

In some embodiments, the method is a method for screening for an NRAS binding compound, the method comprising:

(a) contacting a sample comprising: (i) an NRAS protein; and (ii) an NRAS binding agent comprising an NRAS binding moiety and a functional element, with a candidate NRAS binding compound; and

(b) detecting or quantifying a signal from the functional element. In some embodiments, the NRAS binding compound binds the NRAS protein and detectably alters the signal from the functional element.

In the disclosed systems and methods, the RAS binding agent is a RAS binding agent disclosed herein (e.g., a KRAS binding agent comprising a KRAS binding moiety and a functional element, an HRAS binding agent comprising an HRAS binding moiety and a functional element, or an NRAS binding agent comprising an NRAS binding moiety and a functional element). Exemplary RAS binding agents include compounds of formula (I).

In some embodiments, as discussed herein, the RAS protein in the system or the method is a RAS variant. In some embodiments, the KRAS protein in the system or the method is a KRAS variant, such as a variant selected from KRAS^{G12C}, KRAS^{G12D}, KRAS^{G12V}, KRAS^{Q61R}, KRAS^{Q61H}, KRAS^{Q61L}, and KRAS^{G13D}. In some embodiments, the KRAS protein in the system or method is KRAS^{G12C}. In some embodiments, the HRAS protein in the system or the method is an HRAS variant, such as a variant selected from HRAS^{G12S} and HRAS^{G12V}. In some embodiments, the NRAS protein in the system or method is NRAS^{G12D} or NRAS^{Q61R}.

In some embodiments, as discussed above, the RAS protein (e.g., KRAS, HRAS, or NRAS protein, or a variant thereof) is expressed within the system or the sample. In some embodiments, the RAS protein (or a variant thereof) is provided in a cell-free system or sample, e.g., an in vitro sample or a purified protein sample. In some embodiments, the RAS protein (or a variant thereof) is provided in a cell-free sample for a probe displacement assay (e.g., a probe displacement assay based on fluorescence resonance energy transfer (FRET), bioluminescence energy transfer (BRET), fluorescence polarization (FP), radioligand binding, or the like.

In some embodiments, the RAS protein (e.g., KRAS, HRAS, or NRAS protein, or a variant thereof) is provided/expressed in the systems and methods disclosed herein as a fusion with a bioluminescent reporter, such as a luciferase (e.g., an Oplophorus luciferase). In particular embodiments, the RAS protein or variant thereof is provided/expressed as a fusion with a bioluminescent polypeptide and/or a component of a bioluminescent complex based on NanoLuc® luciferase (SEQ ID NO: 23 and SEQ ID NO: 24), NanoBiT, or NanoTrip. In other particular embodiments, the RAS protein is expressed/provided as a fusion with a component of a bioluminescent complex, including but not limited to NanoBiT®, NanoTrip, and/or the peptide and polypeptide components of bioluminescent complexes described herein. In some embodiments, such peptide and/or polypeptide components of bioluminescent complexes are linked (e.g., fused, chemically linked, etc.) to the RAS protein for use in the methods and systems described herein. For example, in some embodiments, the RAS protein is expressed/provided as a fusion with LgBiT (SEQ ID NO: 25), SmBiT (SEQ ID NO: 26), LgTrip 3092 (SEQ ID NO: 27), LgTrip 3546 (SEQ ID NO: 28), LgTrip 2098 (SEQ ID NO: 29), or SmTrip9 (SEQ ID NO: 30). In embodiments using a RAS fusion with a bioluminescent reporter, the methods may further comprise a step of contacting the sample with a substrate for the bioluminescent reporter. In some embodiments, the substrate for the bioluminescent reporter is selected from coelenterazine, a coelenterazine derivative (e.g., coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine (“coelenterazine-hh”), coelenterazine-i, coelenterazine-icp, coelenterazine-v, and 2-methyl coelenterazine), and furimazine. In some embodiments, the substrate for the bioluminescent reporter is furimazine.

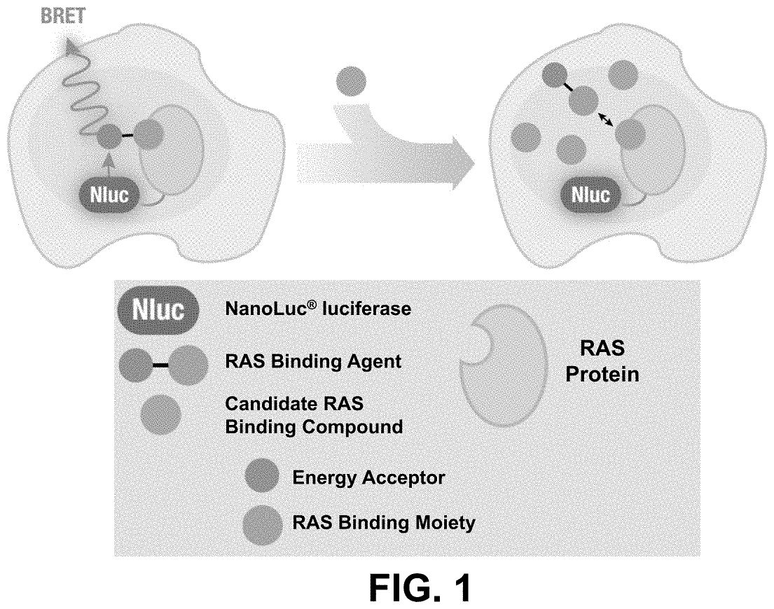

When fusion proteins of a RAS protein (e.g., KRAS, HRAS, or NRAS protein, or a variant thereof) and a bioluminescent reporter are used in the systems and methods described herein, and when the RAS binding agent comprises an energy acceptor (e.g., a fluorophore) as the functional element, the methods may further comprise a step of detecting energy transfer from the bioluminescent reporter to the energy acceptor, if the emission spectrum of the bioluminescent reporter and the excitation spectrum of the energy acceptor overlap. Such a step can identify a RAS binding compound, for example, by detecting a change to the energy transfer upon contact of the sample with the candidate RAS binding compound. An exemplary assay is depicted in FIG. 1, where the RAS protein is expressed as a fusion with NanoLuc® luciferase, and the RAS binding agent comprises a RAS binding moiety and an energy acceptor. When the RAS binding agent binds to the RAS protein, and the sample is contacted with a substrate for the NanoLuc® luciferase (e.g., coelenterazine, a coelenterazine derivative, or furimazine), energy transfer from the NanoLuc® luciferase to the energy acceptor can be detected. If a candidate RAS binding compound binds to the RAS protein, the BRET signal will decrease. As shown in the Examples herein, a loss of BRET signal can be detected even when a candidate RAS binding compound binds to a different site on the RAS protein than the RAS binding agent. For example, in some embodiments, the RAS binding agent binds to the RAS switch I/II site, while the candidate RAS binding compound binds to the switch I/II site or to the switch II site. In other embodiments, the RAS binding agent binds to the RAS switch II site, while the candidate RAS binding compound binds to the switch I/II site or to the switch II site. In some embodiments, candidate RAS binding compounds that have been determined to bind RAS protein (or variants thereof) can be used as RAS binding agents (e.g., linked to a functional element) and screened against other candidate RAS binding compounds.

Accordingly, in some embodiments, provided herein are systems and methods comprising a RAS protein fused to a bioluminescent reporter (e.g., a NanoLuc®-based reporter) and a RAS binding agent comprising an energy acceptor (e.g., a fluorophore) as the detectable element, wherein the emission spectrum of the bioluminescent reporter and the excitation spectrum of the fluorophore overlap, such that engagement (e.g., binding) of the RAS binding agent to the RAS protein can be detected by an increase (e.g., the presence of) BRET between the bioluminescent reporter and the energy acceptor (e.g., a fluorophore). In some embodiments, the engagement (e.g., binding) of a RAS binding compound to the RAS protein can subsequently be detected by a decrease (e.g., the loss of) BRET between the bioluminescent reporter and the energy acceptor (e.g., a fluorophore).

In some embodiments, the bioluminescent reporter is a luciferase having at least 70% sequence identity with SEQ ID NO: 24 (e.g., 70%, 75%, 80%, 85%, 90%, 95%, 96%, 97%, 98%, 99%, 100%, or any range therebetween).

In some embodiments, the systems and methods disclosed herein comprise a first RAS protein fused with a first subunit of a bioluminescent reporter, and a second RAS protein fused with a second subunit of a bioluminescent reporter, wherein the first and second subunits are complementary. When RAS multimeric species form in cells (e.g., dimers), the two complementary bioluminescent reporter subunits come in close proximity to form a functional luciferase that produces a luminescent signal upon reaction with the substrate (e.g., coelenterazine, a coelenterazine derivative, or furimazine). Accordingly, in some embodiments, the first subunit of the bioluminescent reporter has at least 70% sequence identity with SEQ ID NO: 25 (e.g., 70%, 75%, 80%, 85%, 90%, 95%, 96%, 97%, 98%, 99%, 100%, or any range therebetween), and the second subunit of the bioluminescent reporter has at least 90% sequence identity with SEQ ID NO: 26 (e.g., 90%, 91%, 92%, 93%, 94%, 95%, 96%, 97%, 98%, 99%, 100%, or any range therebetween).

The methods disclosed herein can be used with a wide variety of samples. For example, in some embodiments, the sample is selected from a cell, cell lysate, body fluid, tissue, biological sample, in vitro sample, environmental sample, cell-free sample, and purified sample (e.g., a purified protein sample). In some embodiments, the sample comprises cells, such as cells expressing the RAS protein or variant thereof (e.g., KRAS, HRAS, or NRAS protein, or a variant thereof), such as a RAS protein or variant thereof (e.g., KRAS, HRAS, or NRAS protein, or a variant thereof) fused to a bioluminescent reporter.

In some embodiments of the systems and methods disclosed herein, the RAS binding agent is cell-permeable. In some embodiments, such as those using a bioluminescent reporter, the systems and methods further comprise a cell-impermeable inhibitor for the bioluminescent reporter, to ensure that any BRET signal is from live, uncompromised cells.

### V. EXAMPLES

### Example 1

### Synthesis of RAS Binding Agents

### 3-(5,5-difluoro-7-(1H-pyrrol-2-yl)-5H-5λ⁴,6λ⁴-dipyrrolo[1,2-c:2′,1′-f][1,3,2]diazaborinin-3-yl)-N-(2-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)ethyl)propenamide (JRW-2024)

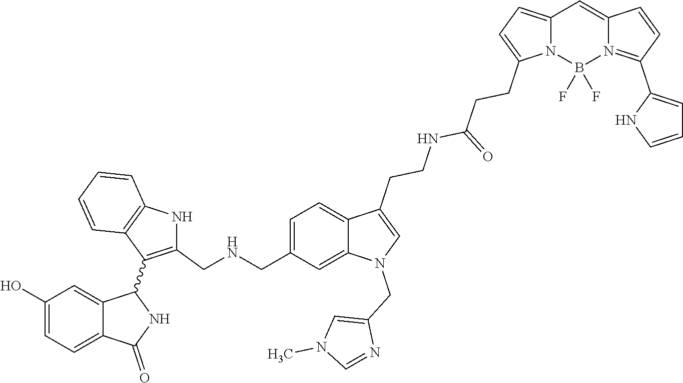

### Step 1. 3-(2-nitrovinyl)-1H-indole-6-carbonitrile (JRW-1991)

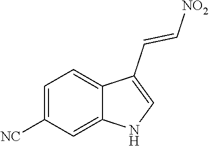 [SMILES: N#Cc1ccc2c(/C=C/[N+](=O)[O-])c[nH]c2c1]

To a suspension of 3-formyl-1H-indole-6-carbonitrile (0.39 g, 2.3 mmol) in nitromethane (10 mL), ammonium acetate (400 mg) was added. The mixture was heated to 85° C. for 18 h. The reaction was cooled resulting in a yellow precipitation. The solid was filtered and washed with methanol/water (1:1) to give crude product (290 mg) as a yellow solid. ESI MS m/z 214 [M+1]+.

### Step 2. 3-(2-nitroethyl)-1H-indole-6-carbonitrile (JRW-1992)

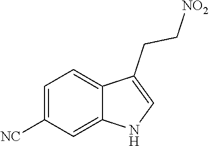 [SMILES: N#Cc1ccc2c(CC[N+](=O)[O-])c[nH]c2c1]

To a solution of 3-(2-nitrovinyl)-1H-indole-6-carbonitrile (290 mg, 1.4 mmol) in tetrahydrofuran/methanol (1:1, 10 mL), sodium borohydride (62 mg, 1.6 mmol) was added. The reaction stirred at rt for 1 h. The mixture was diluted with water and extracted with ethyl acetate. The organic layers were combined, dried with sodium sulfate, filtered, concentrated, and purified with silica gel chromatography to afford the desired product (220 mg, 44% over two steps) as a light yellow solid. ESI MS m/z 214 [M−1]+.

### Step 3. tert-butyl (2-(6-cyano-1H-indol-3-yl)ethyl)carbamate (JRW-2016)

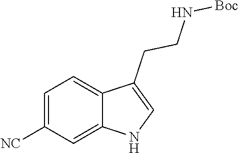 [SMILES: CC(C)(C)OC(=O)NCCc1c[nH]c2cc(C#N)ccc12]

To a solution of 3-(2-nitroethyl)-1H-indole-6-carbonitrile (220 mg, 1.0 mmol) in acetonitrile (20 mL) chilled with an ice bath, diisopropylethylamine (660 mg, 5.1 mmol) and trichlorosilane (484 mg, 3.6 mmol) was added. The reaction stirred at 0° C. for 30 min and rt for 3 h. The reaction was neutralized with a saturated sodium bicarbonate solution (10 mL), and then di-tert-butyl decarbonate (436 mg, 2.0 mmol) was added to the amino intermediate. The mixture stirred at rt for 18 h. The reaction was diluted with acetonitrile, celite was added, concentrated, and purified with silica gel chromatography to afford the desired product (160 mg, 56%) as a white foam. ESI MS m/z 286 [M+1]+.

### Step 4. tert-butyl (2-(6-cyano-1-((1-methyl-1H-imidazol-4-yl)methyl)-1H-indol-3-yl)ethyl)carbamate (JRW-2017)

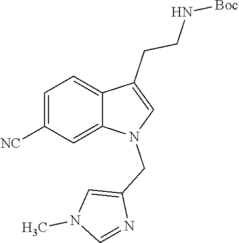 [SMILES: Cn1cnc(Cn2cc(CCNC(=O)OC(C)(C)C)c3ccc(C#N)cc32)c1]

To a solution of tert-butyl (2-(6-cyano-1H-indol-3-yl)ethyl)carbamate (160 mg, 0.56 mmol) in tetrahydrofuran (20 mL) chilled with an ice bath, sodium hydride (45 mg, 1.1 mmol, 60%), 1-(chloromethyl)-1H-imidazole (98 mg, 0.84 mmol), and tetrabutylammonium iodide (20 mg, 0.056 mmol) was added. The mixture stirred at 0° C. for 30 min and rt for 18 h. The reaction was diluted with methanol, celite was added, concentrated, and purified with silica gel chromatography to afford the desired product (120 mg, 56%) as an orange oil. ESI MS m/z 380 [M+1]+.

### Step 5. tert-butyl (2-(6-(aminomethyl)-1-((1-methyl-1H-imidazol-4-yl)methyl)-1H-indol-3-yl)ethyl)carbamate (JRW-2019)

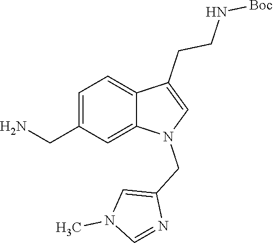 [SMILES: Cn1cnc(Cn2cc(CCNC(=O)OC(C)(C)C)c3ccc(CN)cc32)c1]

To a solution of tert-butyl (2-(6-cyano-1-((1-methyl-1H-imidazol-4-yl)methyl)-1H-indol-3-yl)ethyl)carbamate (120 mg, 0.32 mmol) in ammonia in methanol (7 N, 20 mL), a scoop of Rainey Nickel suspended in water was added. The reaction was charged with hydrogen (60 psi) and stirred at rt for 5 h. After degassing with nitrogen, the mixture was filtered over celite. The filtrate was concentrated and purified with silica gel chromatography to afford the desired product (75 mg, 62%) as a colorless oil. ESI MS m/z 384 [M+1]+.

### Step 6. tert-butyl (2-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)ethyl)carbamate (J-RW-2021)

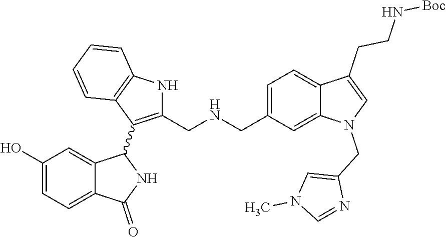 [SMILES: Cn1cnc(Cn2cc(CCNC(=O)OC(C)(C)C)c3ccc(CNCc4[nH]c5ccccc5c4C4NC(=O)c5ccc(O)cc54)cc32)c1]

To a suspension of tert-butyl (2-(6-(aminomethyl)-1-((1-methyl-1H-imidazol-4-yl)methyl)-1H-indol-3-yl)ethyl)carbamate (65 mg, 0.17 mmol) and 3-(6-hydroxy-3-oxoisoindolin-1-yl)-1H-indole-2-carbaldehyde (50 mg, 0.17 mmol) in toluene (10 mL), magnesium sulfate (102 mg, 0.85 mmol) was added. The suspension was heated at 100 C for 18 h. The reaction was cooled, concentrated, and resuspended in methanol (10 mL). The mixture was cooled to 0° C., and sodium borohydride (20 mg, 0.52 mmol) was added. The reduction was warmed to rt and stirred for 1 h. The reaction was diluted with methanol, celite was added, concentrated, and purified with silica gel chromatography to afford the desired product (42 mg, 37%) as an orange solid. ESI MS m/z 660 [M+1]+.

### Step 7. 3-(2-((((3-(2-aminoethyl)-1-((1-methyl-1H-imidazol-4-yl)methyl)-1H-indol-6-yl)methyl)amino)methyl)-1H-indol-3-yl)-5-hydroxyisoindolin-1-one (JRW-2022)

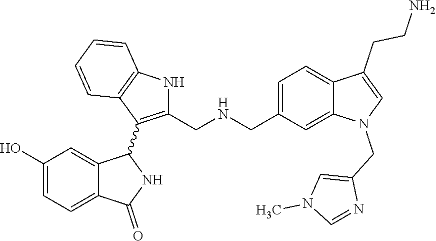 [SMILES: Cn1cnc(Cn2cc(CCN)c3ccc(CNCc4[nH]c5ccccc5c4C4NC(=O)c5ccc(O)cc54)cc32)c1]

To a solution of tert-butyl (2-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)ethyl)carbamate (42 mg, 0.064 mmol) in dichloromethane (10 mL), trifluoroacetic acid (1 mL) was added. The reaction stirred at rt for 2 h. The mixture was concentrated to give crude product as a brown oil. ESI MS m/z 560 [M+1]+.

### Step 8. 3-(5,5-difluoro-7-(1H-pyrrol-2-yl)-5H-5λ⁴,6λ⁴-dipyrrolo[1,2-c:2′,1′-f][1,3,2]diazaborinin-3-yl)-N-(2-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)ethyl)propenamide (JRW-2024)

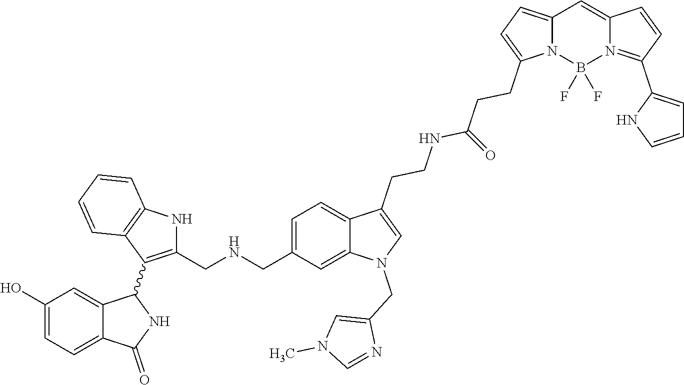

To a solution of 3-(2-((((3-(2-aminoethyl)-1-((1-methyl-1H-imidazol-4-yl)methyl)-1H-indol-6-yl)methyl)amino)methyl)-1H-indol-3-yl)-5-hydroxyisoindolin-1-one (10 mg, 0.018 mmol) in DMF (5 mL), 2,5-dioxopyrrolidin-1-yl 3-(5,5-difluoro-7-(1H-pyrrol-2-yl)-5H-5λ⁴,6λ⁴-dipyrrolo[1,2-c:2′,1′-f][1,3,2]diazaborinin-3-yl)propanoate (7 mg, 0.018 mmol) and diisopropylethylamine (18 mg, 0.14 mmol) was added. The reaction was stirred at rt for 30 min. The mixture was diluted with methanol and purified by reverse phase preparative HPLC to afford the desired product (13 mg, 86%) as a purple solid. ESI MS m/z 871 [M+1]+.

### 1-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-N-(2-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)ethyl)-3,6,9,12-tetraoxapentadecan-15-amide (JRW-2025)

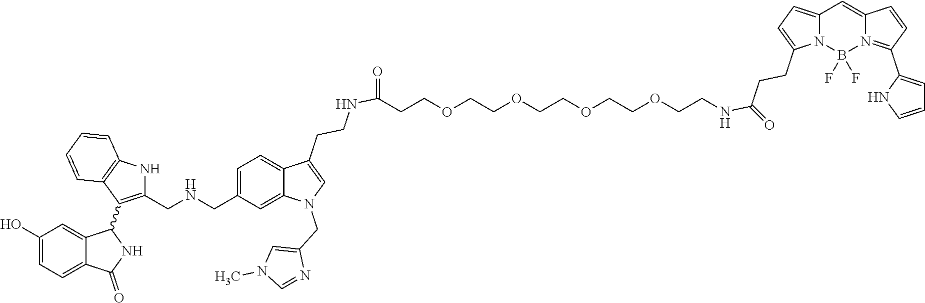

To a solution of 3-(2-((((3-(2-aminoethyl)-1-((1-methyl-1H-imidazol-4-y)methyl)-1H-indol-6-yl)methyl)amino)methyl)-1H-indol-3-yl)-5-hydroxyisoindolin-1-one (10 mg, 0.018 mmol) in DMF (5 mL), 2,5-dioxopyrrolidin-1-yl 1-(5,5-difluoro-7-(1H-pyrrol-2-yl)-5H-5λ⁴,6λ⁴-dipyrrolo[1,2-c:2′,1′-f][1,3,2]diazaborinin-3-yl)-3-oxo-7,10,13,16-tetraoxa-4-azanonadecan-19-oate (12 mg, 0.018 mmol) and diisopropylethylamine (18 mg, 0.14 mmol) was added. The reaction was stirred at rt for 30 min. The mixture was diluted with methanol and purified by reverse phase preparative HPLC to afford the desired product (13 mg, 65%) as a purple solid. ESI MS m/z 1118 [M+1]+.

### 3-(5,5-difluoro-7-(1H-pyrrol-2-yl)-5H-5λ⁴,6λ⁴-dipyrrolo[1,2-c:2′,1′-f][1,3,2]diazaborinin-3-yl)-N-(2-(4-((6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)methyl)piperazin-1-yl)ethyl)propenamide (JRW-2029)

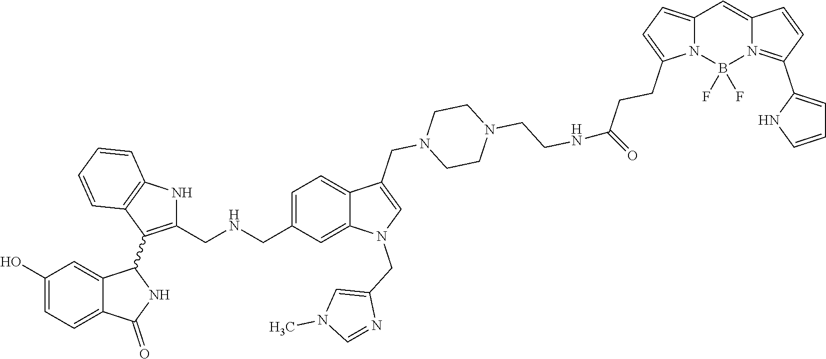

### Step 1. Tert-butyl (2-(4-((6-cyano-1H-indol-3-yl)methyl)piperazin-1-yl)ethyl)carbamate (JRW-1987)

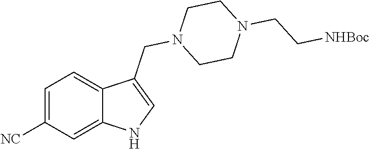 [SMILES: CC(C)(C)OC(=O)NCCN1CCN(Cc2c[nH]c3cc(C#N)ccc23)CC1]

To a solution of 3-formyl-1H-indole-6-carbonitrile (477 mg, 2.8 mmol) in tetrahydrofuran (20 mL), tert-butyl (2-(piperazin-1-yl)ethyl)carbamate (771 mg, 3.4 mmol) and sodium cyanoborohydride (1.5 g, 7.0 mmol) was added. The reaction was stirred at 40° C. for 18 h. The mixture was diluted with water and extracted with chloroform/isopropanol (3:1). The organic layers were combined, dried with sodium sulfate, filtered, concentrated, and purified with silica gel chromatography to afford the desired product (910 mg, 85%) as a colorless oil. ESI MS m/z 384 [M+1]+.

### Step 2. Tert-butyl (2-(4-((6-cyano-1-((1-methyl-1H-imidazol-4-yl)methyl)-1H-indol-3-yl)methyl)piperazin-1-yl ethyl)carbamate (JRW-2011)

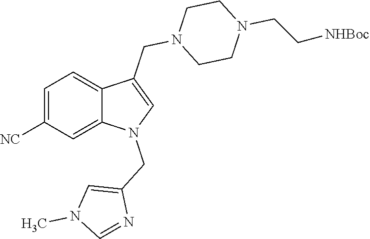 [SMILES: Cn1cnc(Cn2cc(CN3CCN(CCNC(=O)OC(C)(C)C)CC3)c3ccc(C#N)cc32)c1]

To a solution of tert-butyl (2-(4-((6-cyano-1H-indol-3-yl)methyl)piperazin-1-yl)ethyl)carbamate (200 mg, 0.52 mmol) in tetrahydrofuran (20 mL) chilled with an ice bath, sodium hydride (42 mg, 1.0 mmol, 60%), 1-(chloromethyl)-1H-imidazole (74 mg, 0.57 mmol), and tetrabutylammonium iodide (20 mg, 0.052 mmol) was added. The mixture stirred at 0° C. for 30 min and rt for 6 h. The mixture was diluted with water (pH 10) and extracted with chloroform/isopropanol (3:1). The organic layers were combined, dried with sodium sulfate, filtered, concentrated, and purified with silica gel chromatography to afford the desired product (187 mg, 75%) as a colorless oil. ESI MS m/z 478 [M+1]+.

### Step 3. Tert-butyl (2-(4-((6-(aminomethyl)-1-((1-methyl-1H-imidazol-4-yl)methyl)-1H-indol-3-yl)methyl)piperazin-1-yl)ethyl)carbamate (JRW-2013)

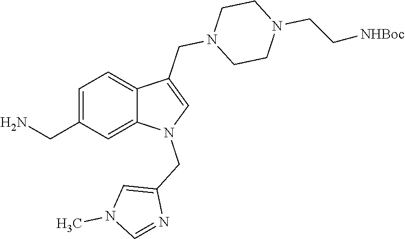 [SMILES: Cn1cnc(Cn2cc(CN3CCN(CCNC(=O)OC(C)(C)C)CC3)c3ccc(CN)cc32)c1]

To a solution of tert-butyl (2-(4-((6-cyano-1H-indol-3-yl)methyl)piperazin-1-yl)ethyl)carbamate (187 mg, 0.39 mmol) in ammonia in methanol (7 N, 20 mL), a scoop of Rainey Nickel suspended in water was added. The reaction was charged with hydrogen (60 psi) and stirred at rt for 5 h. After degassing with nitrogen, the mixture was filtered over celite. The filtrate was concentrated to afford the desired product (170 mg, crude) as a colorless wax. ESI MS m/z 482 [M+1]+.

### Step 4. tert-butyl (2-(4-((6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)methyl)piperazin-1-yl)ethyl)carbamate (JRW-2023)

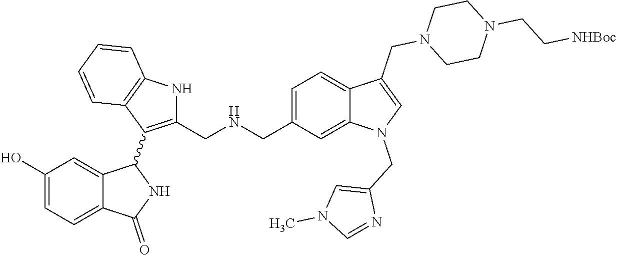 [SMILES: Cn1cnc(Cn2cc(CN3CCN(CCNC(=O)OC(C)(C)C)CC3)c3ccc(CNCc4[nH]c5ccccc5c4C4NC(=O)c5ccc(O)cc54)cc32)c1]

To a suspension of tert-butyl (2-(4-((6-(aminomethyl)-1-((1-methyl-1H-imidazol-4-yl)methyl)-1H-indol-3-yl)methyl)piperazin-1-yl)ethyl)carbamate (65 mg, 0.14 mmol) and 3-(6-hydroxy-3-oxoisoindolin-1-yl)-1H-indole-2-carbaldehyde (40 mg, 0.14 mmol) in toluene (10 mL), magnesium sulfate (82 mg, 0.68 mmol) was added. The suspension was heated at 100° C. for 18 h. The reaction was cooled, concentrated, and resuspended in methanol (10 mL). The mixture was cooled to 0° C., and sodium borohydride (16 mg, 0.41 mmol) was added. The reduction was warmed to rt and stirred for 1 h. The reaction was diluted with methanol, celite was added, concentrated, and purified with silica gel chromatography to afford the desired product (35 mg, 34%) as a brown solid. ESI MS m/z 758 [M+1]+.

### Step 5. 3-(2-((((3-((4-(2-aminoethyl)piperazin-1-yl)methyl)-1-((1-methyl-1H-imidazol-4-yl)methyl)-1H-indol-6-yl)methyl)amino)methyl)-1H-indol-3-yl)-5-hydroxyisoindolin-1-one (JRW-2026)

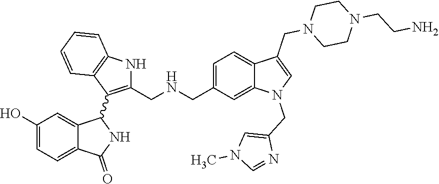 [SMILES: Cn1cnc(Cn2cc(CN3CCN(CCN)CC3)c3ccc(CNCc4[nH]c5ccccc5c4C4NC(=O)c5ccc(O)cc54)cc32)c1]

To a solution of tert-butyl (2-(4-((6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)methyl)piperazin-1-yl)ethyl)carbamate (25 mg, 0.046 mmol) in dichloromethane (10 mL), trifluoroacetic acid (1 mL) was added. The reaction stirred at rt for 4 h. The mixture was concentrated to give crude product as a brown oil. ESI MS m/z 658 [M+1]+.

### Step 6. 3-(5,5-difluoro-7-(1H-pyrrol-2-yl)-5H-5λ⁴,6λ⁴-dipyrrolo[1,2-c:2′,1′-f][1,3,2]diazaborinin-3-yl)-N-(2-(4-((6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)methyl)piperazin-1-yl)ethyl)propenamide (JRW-2029)

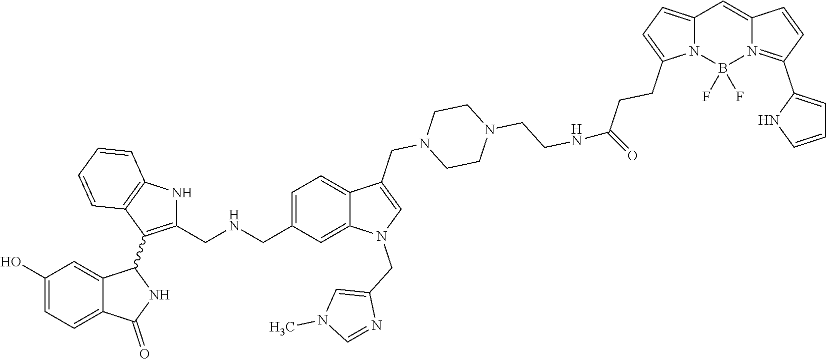

To a solution of 3-(2-((((3-((4-(2-aminoethyl)piperazin-1-yl)methyl)-1-((1-methyl-1H-imidazol-4-yl)methyl)-1H-indol-6-yl)methyl)amino)methyl)-1H-indol-3-yl)-5-hydroxyisoindolin-1-one (10 mg, 0.015 mmol) in DMF (5 mL), 2,5-dioxopyrrolidin-1-yl 3-(5,5-difluoro-7-(1H-pyrrol-2-yl)-5H-5λ⁴,6λ⁴-dipyrrolo[1,2-c:2′,1′-f][1,3,2]diazaborinin-3-yl)propanoate (6 mg, 0.015 mmol) and diisopropylethylamine (16 mg, 0.12 mmol) was added. The reaction was stirred at rt for 30 min. The mixture was diluted with methanol and purified by reverse phase preparative HPLC to afford the desired product (13 mg, 86%) as a purple solid. ESI MS m/z 969 [M+1]+.

### 1-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-N-(2-(4-((6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)methyl)piperazin-1-yl)ethyl)-3,6,9,12-tetraoxapentadecan-15-amide (JRW-2030)

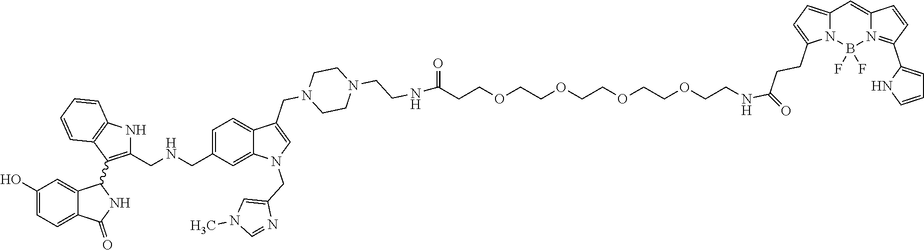

To a solution of 3-(2-((((3-((4-(2-aminoethyl)piperazin-1-yl)methyl)-1-((1-methyl-1H-imidazol-4-yl)methyl)-1H-indol-6-yl)methyl)amino)methyl)-1H-indol-3-yl)-5-hydroxyisoindolin-1-one (10 mg, 0.015 mmol) in DMF (5 mL), 2,5-dioxopyrrolidin-1-yl 1-(5,5-difluoro-7-(1H-pyrrol-2-yl)-5H-5λ⁴,6λ⁴-dipyrrolo[1,2-c:2′,1′-f][1,3,2]diazaborinin-3-yl)-3-oxo-7,10,13,16-tetraoxa-4-azanonadecan-19-oate (10 mg, 0.015 mmol) and diisopropylethylamine (16 mg, 0.12 mmol) was added. The reaction was stirred at rt for 30 min. The mixture was diluted with methanol and purified by reverse phase preparative HPLC to afford the desired product (13 mg, 72%) as a purple solid. ESI MS m/z 1216 [M+1]+.

### 1-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-N-(2-(3-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)propanamido)ethyl)-3,6,9,12-tetraoxapentadecan-15-amide (JRW-2038)

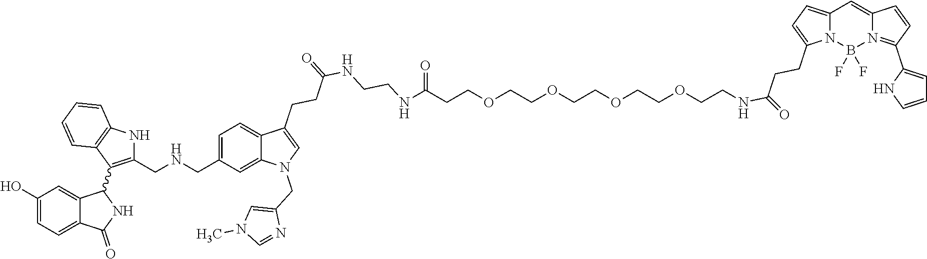

### Step 1. Tert-butyl 3-(6-cyano-1H-indol-3-yl)acrylate (JRW-1995)

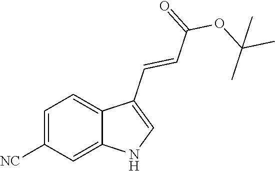 [SMILES: CC(C)(C)OC(=O)/C=C/c1c[nH]c2cc(C#N)ccc12]

To a solution of 3-formyl-1H-indole-6-carbonitrile (200 mg, 1.2 mmol) in acetonitrile (10 mL) was heated to 80° C. tert-butyl 2-(triphenyl-15-phosphaneylidene)acetate (1.1 g, 2.9 mmol) was added in portions. The reaction was stirred at 80° C. for 18 h. The reaction was diluted with methanol, celite was added, concentrated, and purified with silica gel chromatography to afford the desired product (125 mg, 40%) as a white solid. ESI MS m/z 269 [M+1]+.

### Step 2. Tert-butyl 3-(6-cyano-1H-indol-3-yl)propanoate (JRW-1997)

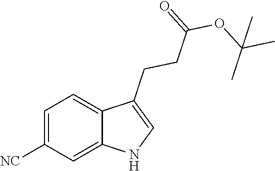 [SMILES: CC(C)(C)OC(=O)CCc1c[nH]c2cc(C#N)ccc12]

To a solution of tert-butyl 3-(6-cyano-1H-indol-3-yl)acrylate (120 mg, 0.45 mmol) in methanol/ethyl acetate (1:1, 10 mL), palladium on carbon (10 mg) was added. Under a hydrogen balloon, the reaction stirred at rt for 2 h. After degassing with nitrogen, the mixture was filtered over celite. The filtrate was concentrated and chromatographed to afford the desired product (105 mg, 87%) as a light red solid. ESI MS m/z 271 [M+1]+.

### Step 3. Tert-butyl 3-(6-cyano-1-((1-methyl-1H-imidazol-4-yl)methyl)-1H-indol-3-yl)propanoate (JRW-2020)

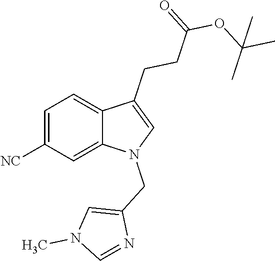 [SMILES: Cn1cnc(Cn2cc(CCC(=O)OC(C)(C)C)c3ccc(C#N)cc32)c1]

To a solution of tert-butyl 3-(6-cyano-1H-indol-3-yl)propanoate (105 mg, 0.39 mmol) in tetrahydrofuran (20 mL) chilled with an ice bath, sodium hydride (23 mg, 0.58 mmol, 60%), 1-(chloromethyl)-1H-imidazole (68 mg, 0.58 mmol), and tetrabutylammonium iodide (14 mg, 0.039 mmol) was added. The mixture stirred at 0° C. for 30 min and 60° C. for 3 h. The reaction was diluted with methanol, celite was added, concentrated, and purified with silica gel chromatography to afford the desired product as a colorless oil. ESI MS m/z 365 [M+1]+.

### Step 4. Tert-butyl 3-(6-(aminomethyl)-1-((1-methyl-1H-imidazol-4-yl)methyl)-1H-indol-3-yl)propanoate (JRW-2027)

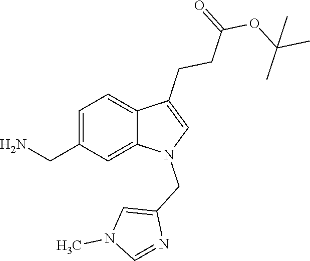 [SMILES: Cn1cnc(Cn2cc(CCC(=O)OC(C)(C)C)c3ccc(CN)cc32)c1]

To a solution of tert-butyl 3-(6-cyano-1-((1-methyl-1H-imidazol-4-yl)methyl)-1H-indol-3-yl)propanoate (0.39 mmol) in ammonia in methanol (7 N, 20 mL), a scoop of Rainey Nickel suspended in water was added. The reaction was charged with hydrogen (60 psi) and stirred at rt for 4 h. After degassing with nitrogen, the mixture was filtered over celite. The filtrate was concentrated and purified with silica gel chromatography to afford the desired product (90 mg, 63% over two steps) as a colorless oil. ESI MS m/z 369 [M+1]+.

### Step 5. tert-butyl 3-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)propanoate (JRW-2031)

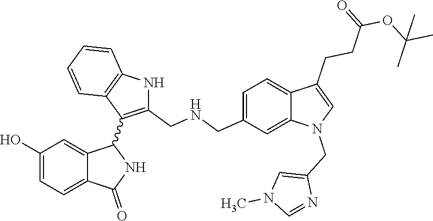 [SMILES: Cn1cnc(Cn2cc(CCC(=O)OC(C)(C)C)c3ccc(CNCc4[nH]c5ccccc5c4C4NC(=O)c5ccc(O)cc54)cc32)c1]

To a suspension of tert-butyl 3-(6-(aminomethyl)-1-((1-methyl-1H-imidazol-4-yl)methyl)-1H-indol-3-yl)propanoate (90 mg, 0.25 mmol) and 3-(6-hydroxy-3-oxoisoindolin-1-yl)-1H-indole-2-carbaldehyde (75 mg, 0.25 mmol) in toluene (10 mL), magnesium sulfate (200 mg) was added. The suspension was heated at 100° C. for 6 h. The reaction was cooled, concentrated, and resuspended in methanol (10 mL). The mixture was cooled to 0° C., and sodium borohydride (29 mg, 0.77 mmol) was added. The reduction was warmed to rt and stirred for 1 h. The reaction was diluted with methanol, celite was added, concentrated, and purified with silica gel chromatography to afford the desired product (85 mg, 52%) as a brown solid. ESI MS m/z 645 [M+1]+.

### Step 6. 3-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)propanoic acid (JRW-2034)

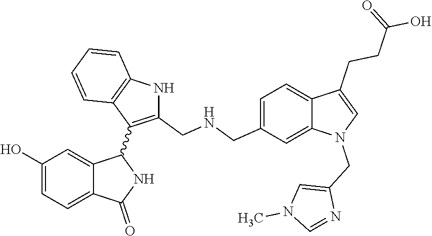 [SMILES: Cn1cnc(Cn2cc(CCC(=O)O)c3ccc(CNCc4[nH]c5ccccc5c4C4NC(=O)c5ccc(O)cc54)cc32)c1]

To a solution of tert-butyl 3-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)propanoate (85 mg, 0.13 mmol) in dichloromethane (10 mL), trifluoroacetic acid (1 mL) was added. The reaction stirred at rt for 2 h. The mixture was concentrated to give crude product as a brown oil. ESI MS m/z 589 [M+1]+.

### Step 7. tert-butyl (2-(3-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)propanamido)ethyl)carbamate (JRW-2035)

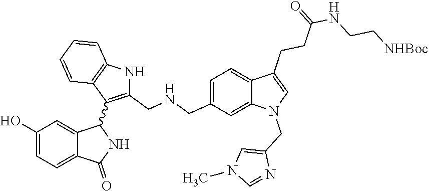 [SMILES: Cn1cnc(Cn2cc(CCC(=O)NCCNC(=O)OC(C)(C)C)c3ccc(CNCc4[nH]c5ccccc5c4C4NC(=O)c5ccc(O)cc54)cc32)c1]

To a solution of 3-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)propanoic acid (0.13 mmol) in DMF (5 mL), N,N,N′,N′-tetramethyl-O—(N-succinimidyl)uronium tetrafluoroborate (45 mg, 0.16 mmol), diisopropylethylamine (51 mg, 0.39 mmol), and tert-butyl (2-aminoethyl)carbamate (42 mg, 0.26 mmol) was added. The reaction stirred at rt for 18 h. The reaction was diluted with methanol, celite was added, concentrated, and purified with silica gel chromatography to afford the desired product (20 mg, 21% over two steps) as a light brown solid. ESI MS m/z 731 [M+1]+.

### Step 8. N-(2-aminoethyl)-3-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)propenamide (JRW-2036)

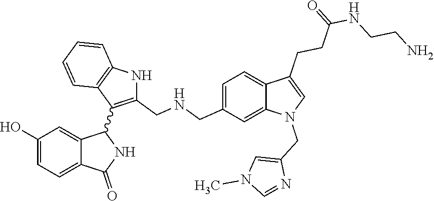 [SMILES: Cn1cnc(Cn2cc(CCC(=O)NCCN)c3ccc(CNCc4[nH]c5ccccc5c4C4NC(=O)c5ccc(O)cc54)cc32)c1]

To a solution of tert-butyl (2-(3-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)propanamido)ethyl)carbamate (20 mg, 0.027 mmol) in dichloromethane (10 mL), trifluoroacetic acid (1 mL) was added. The reaction stirred at rt for 2 h. The mixture was concentrated to give crude product as a brown oil. ESI MS m/z 631 [M+1]+.

### Step 9. 1-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-N-(2-(3-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)propanamido)ethyl)-3,6,9,12-tetraoxapentadecan-15-amide (JRW-2038)

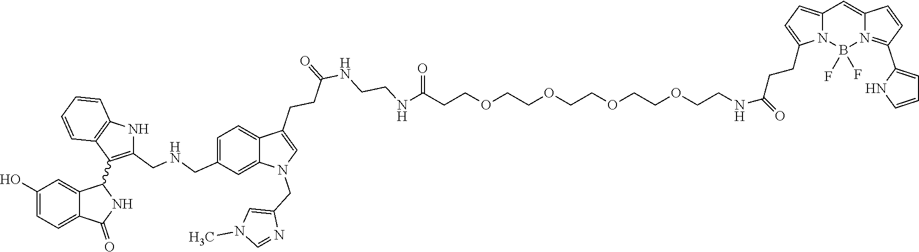

To a solution of N-(2-aminoethyl)-3-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)propenamide (0.027 mmol) in DMF (5 mL), 2,5-dioxopyrrolidin-1-yl 1-(5,5-difluoro-7-(1H-pyrrol-2-yl)-5H-5λ⁴,6λ⁴-dipyrrolo[1,2-c:2′,1′-f][1,3,2]diazaborinin-3-yl)-3-oxo-7,10,13,16-tetraoxa-4-azanonadecan-19-oate (9 mg, 0.013 mmol) and diisopropylethylamine (28 mg, 0.22 mmol) was added. The reaction was stirred at rt for 30 min. The mixture was diluted with methanol and purified by reverse phase preparative HPLC to afford the desired product (10 mg, 64%) as a purple solid. ESI MS m/z 1189 [M+1]+.

### 4-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-N-(3-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)propyl)butanamide (JRW-2093)

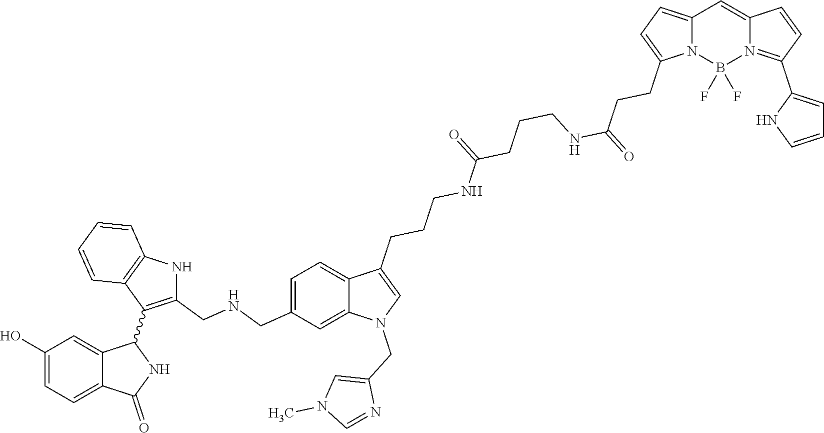

### Step 1. Tert-butyl 3-bromo-6-cyano-1H-indole-1-carboxylate (JRW-2072)

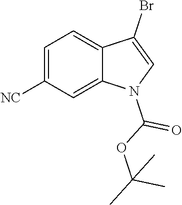 [SMILES: CC(C)(C)OC(=O)n1cc(Br)c2ccc(C#N)cc21]

To a solution of 3-bromo-1H-indole-6-carbonitrile (1.0 g, 4.5 mmol) in dichloromethane (50 mL), di-tert-butyl decarbonate (1.2 g, 5.4 mmol), diisopropylethylamine (1.8 g, 13.6 mmol), and N,N-dimethylpyridin-4-amine (0.055 g, 0.45 mmol was added). The reaction stirred at rt for 2 h. The mixture was diluted with dichloromethane, poured into water with HCl, and the layers separated. The organic layers were combined, dried with sodium sulfate, filtered, concentrated, and purified with silica gel chromatography to afford the desired product (1.3 g, 89%) as a white solid. ESI MS m/z 322 [M+1]+.

### Step 2. Tert-butyl 3-(3-((tert-butoxycarbonyl)amino)prop-1-yn-1-yl)-6-cyano-1H-indole-1-carboxylate (JRW-2074)

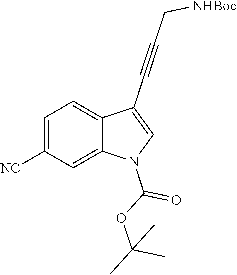 [SMILES: CC(C)(C)OC(=O)NCC#Cc1cn(C(=O)OC(C)(C)C)c2cc(C#N)ccc12]

To a solution of tert-butyl 3-bromo-6-cyano-1H-indole-1-carboxylate (1.3 g, 4.0 mmol) in tetrahydrofuran (30 mL), tert-butyl prop-2-yn-1-ylcarbamate (1.6 g, 10.2 mmol), bis(triphenylphosphine)palladium(II) dichloride (0.28 g, 0.40 mmol), copper iodide (0.077 g, 0.40 mmol), and triphenylphosphine (0.42 g, 1.6 mmol) was added. The solution was degassed and purged with nitrogen. Diethylamine (4.4 g, 60.7 mmol) was added, and the reaction was stirred at 60° C. for 5 h. The reaction was diluted with ethyl acetate, celite was added, concentrated, and purified with silica gel chromatography to afford the desired product (0.80 g, 50%) as a light brown foam. ESI MS m/z 396 [M+1]+.

### Step 3. Tert-butyl (3-(6-cyano-1H-indol-3-yl)prop-2-yn-1-yl)carbamate (JRW-2076)

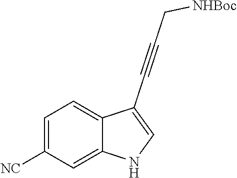 [SMILES: CC(C)(C)OC(=O)NCC#Cc1c[nH]c2cc(C#N)ccc12]

To a solution of tert-butyl 3-(3-((tert-butoxycarbonyl)amino)prop-1-yn-1-yl)-6-cyano-1H-indole-1-carboxylate (300 mg, 0.76 mmol) in methanol/tetrahydrofuran (1:1, 20 mL), cesium carbonate (494 mg, 1.5 mol) was added. The reaction stirred at rt for 30 min. The solvent was evaporated to give crude product as a yellow solid. ESI MS m/z 296 [M+1]+.

### Step 4. Tert-butyl (3-(6-cyano-1-((1-methyl-1H-imidazol-4-yl)methyl)-1H-indol-3-yl)prop-2-yn-1-yl)carbamate (JRW-2077)

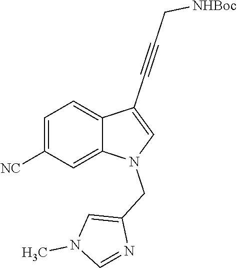 [SMILES: Cn1cnc(Cn2cc(C#CCNC(=O)OC(C)(C)C)c3ccc(C#N)cc32)c1]

To a solution of tert-butyl (3-(6-cyano-1H-indol-3-yl)prop-2-yn-1-yl)carbamate (0.76 mmol) in DMF (20 mL) chilled with an ice bath, sodium hydride (45 mg, 1.1 mmol, 60%), 1-(chloromethyl)-1H-imidazole (148 mg, 1.1 mmol), and tetrabutylammonium iodide (28 mg, 0.076 mmol) was added. The mixture stirred at 0° C. for 30 min and rt for 6 h. The reaction was diluted with ethyl acetate and washed with water. The organic layers were combined, dried with sodium sulfate, filtered, concentrated, and purified with silica gel chromatography to afford the desired product (130 mg, 44%) as an orange oil. ESI MS m/z 390 [M+1]+.

### Step 5. Tert-butyl (3-(6-(aminomethyl)-1-((1-methyl-1H-imidazol-4-yl)methyl)-1H-indol-3-yl)propyl)carbamate (JRW-2080)

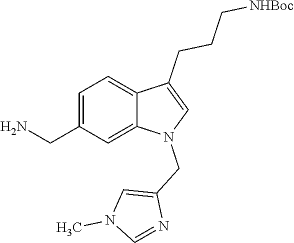 [SMILES: Cn1cnc(Cn2cc(CCCNC(=O)OC(C)(C)C)c3ccc(CN)cc32)c1]

To a solution of tert-butyl (3-(6-cyano-1-((1-methyl-1H-imidazol-4-yl)methyl)-1H-indol-3-yl)prop-2-yn-1-yl)carbamate (130 mg, 0.33 mmol) in ammonia in methanol (7 N, 20 mL), a scoop of Rainey Nickel suspended in water was added. The reaction was charged with hydrogen (60 psi) and stirred at rt for 18 h. After degassing with nitrogen, the mixture was filtered over celite. The filtrate was concentrated to afford the desired product (120 mg, crude) as a light brown solid. ESI MS m/z 394 [M+1]+.

### Step 6. Tert-butyl (3-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)propyl)carbamate (JRW-2083)

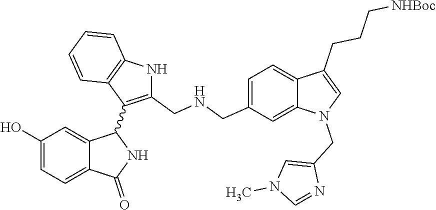 [SMILES: Cn1cnc(Cn2cc(CCCNC(=O)OC(C)(C)C)c3ccc(CNCc4[nH]c5ccccc5c4C4NC(=O)c5ccc(O)cc54)cc32)c1]

To a suspension of tert-butyl (3-(6-(aminomethyl)-1-((1-methyl-1H-imidazol-4-yl)methyl)-1H-indol-3-yl)propyl)carbamate (120 mg, 0.30 mmol) and 3-(6-hydroxy-3-oxoisoindolin-1-yl)-1H-indole-2-carbaldehyde (75 mg, 0.25 mmol) in toluene (10 mL), magnesium sulfate (200 mg) was added. The suspension was heated at 100° C. for 18 h. The reaction was cooled, concentrated, and resuspended in methanol (10 mL). The mixture was cooled to 0° C., and sodium borohydride (29 mg, 0.77 mmol) was added. The reduction was warmed to rt and stirred for 2 h. The reaction was diluted with methanol, celite was added, concentrated, and purified with silica gel chromatography to afford the desired product (40 mg, 23%) as a light brown solid. ESI MS m/z 674 [M+1]+.

### Step 7. 3-(2-((((3-(3-aminopropyl)-1-((1-methyl-1H-imidazol-4-yl)methyl)-1H-indol-6-yl)methyl)amino)methyl)-1H-indol-3-yl)-5-hydroxyisoindolin-1-one (JRW-2090)

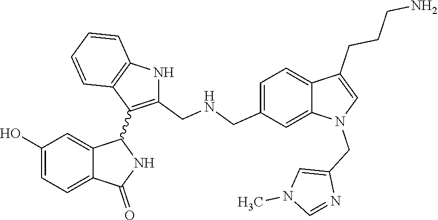 [SMILES: Cn1cnc(Cn2cc(CCCN)c3ccc(CNCc4[nH]c5ccccc5c4C4NC(=O)c5ccc(O)cc54)cc32)c1]

To a solution of tert-butyl (3-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)propyl)carbamate (40 mg, 0.060 mmol) in dichloromethane (10 mL), trifluoroacetic acid (1 mL) was added. The reaction stirred at rt for 2 h. The mixture was concentrated to give crude product as a brown oil. ESI MS m/z 574 [M+1]+.

### Step 8. 4-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-N-(3-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)propyl)butanamide (JRW-2093)

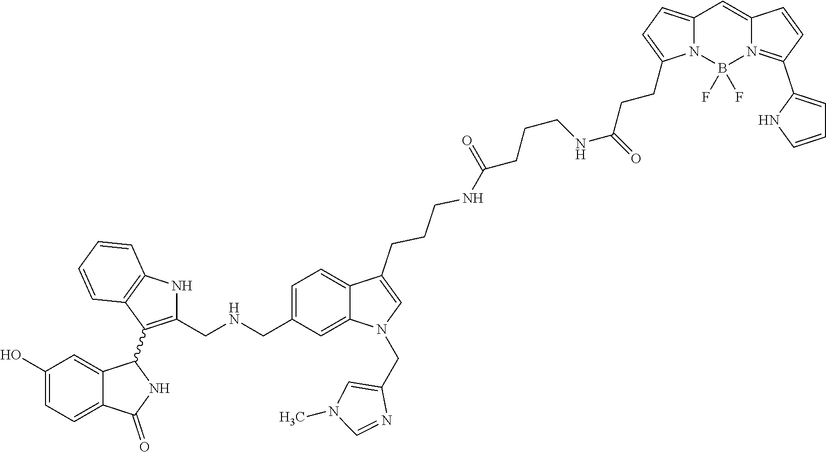

To a solution of 3-(2-((((3-(3-aminopropyl)-1-((1-methyl-1H-imidazol-4-yl)methyl)-1H-indol-6-yl)methyl)amino)methyl)-1H-indol-3-yl)-5-hydroxyisoindolin-1-one (15 mg, 0.026 mmol) in DMF (5 mL), 2,5-dioxopyrrolidin-1-yl 4-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)butanoate (13 mg, 0.026 mmol) and diisopropylethylamine (28 mg, 0.22 mmol) was added. The reaction was stirred at rt for 2 h. The mixture was diluted with methanol and purified by reverse phase preparative HPLC to afford the desired product (9 mg, 36%) as a purple solid. ESI MS m/z 970 [M+1]+.

### 1-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-N-(3-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)propyl)-3,6,9,12-tetraoxapentadecan-15-amide (JRW-2094)

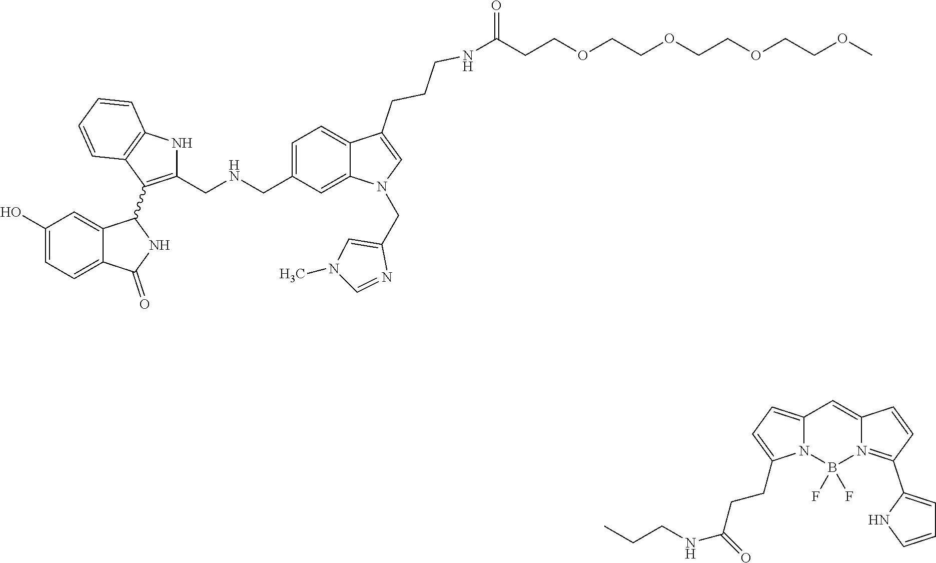

To a solution of 3-(2-((((3-(3-aminopropyl)-1-((1-methyl-1H-imidazol-4-yl)methyl)-1H-indol-6-yl)methyl)amino)methyl)-1H-indol-3-yl)-5-hydroxyisoindolin-1-one (15 mg, 0.026 mmol) in DMF (5 mL), 2,5-dioxopyrrolidin-1-yl 1-(5,5-difluoro-7-(1H-pyrrol-2-yl)-5H-5λ⁴,6λ⁴-dipyrrolo[1,2-c:2′,1′-f][1,3,2]diazaborinin-3-yl)-3-oxo-7,10,13,16-tetraoxa-4-azanonadecan-19-oate (9 mg, 0.013 mmol) and diisopropylethylamine (13 mg, 0.10 mmol) was added. The reaction was stirred at rt for 30 min. The mixture was diluted with methanol and purified by reverse phase preparative HPLC to afford the desired product (5 mg, 35%) as a purple solid. ESI MS m/z 1132 [M+1]+.

### N-(2-(1-benzyl-6-((((3-(6-hydroxy-3-oxoisoindolin-1-yl)-1H-indol-2-yl)methyl)amino)methyl)-1H-indol-3-yl)ethyl)-1-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-3,6,9,12-tetraoxapentadecan-15-amide (JRW-2095)

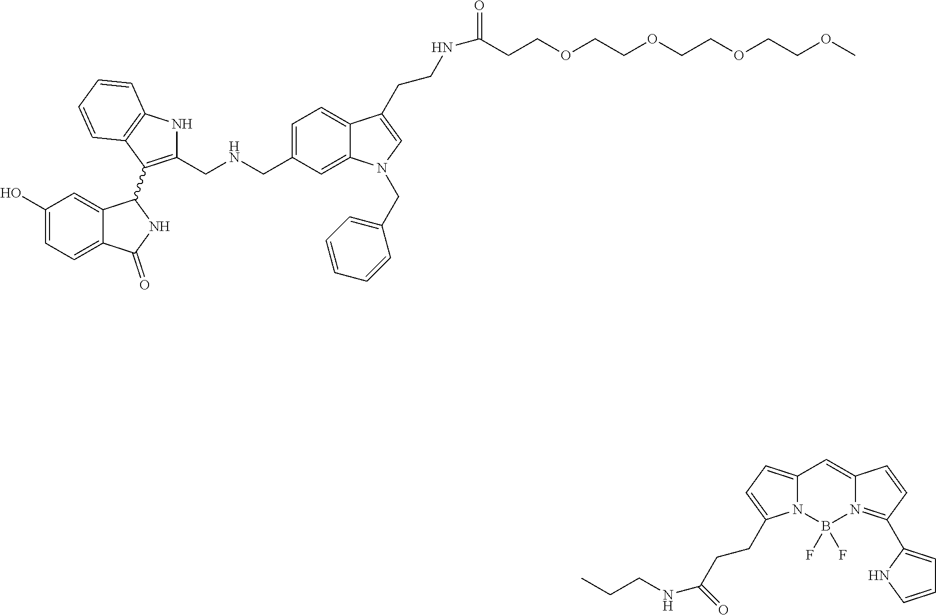

### Step 1. Tert-butyl (2-(1-benzyl-6-cyano-1H-indol-3-yl)ethyl)carbamate (JRW-2079)

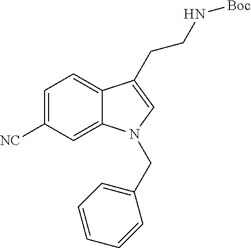 [SMILES: CC(C)(C)OC(=O)NCCc1cn(Cc2ccccc2)c2cc(C#N)ccc12]

To a solution of tert-butyl (2-(6-cyano-1H-indol-3-yl)ethyl)carbamate (550 mg, 1.9 mmol) in DMF (20 mL) chilled with an ice bath, sodium hydride (154 mg, 3.8 mmol, 60%), benzyl chloride (366 mg, 2.9 mmol), and tetrabutylammonium iodide (71 mg, 0.19 mmol) was added. The mixture stirred at 0° C. for 30 min and rt for 8 h. The reaction was diluted with ethyl acetate and washed with water. The organic layers were combined, dried with sodium sulfate, filtered, concentrated, and purified with silica gel chromatography to afford the desired product (445 mg, 61%) as a light brown foam. ESI MS m/z 376 [M+1]+.

### Step 2. Tert-butyl (2-(6-(aminomethyl)-1-benzyl-1H-indol-3-yl)ethyl)carbamate (JRW-2084)

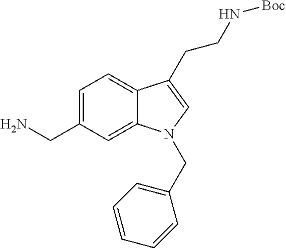 [SMILES: CC(C)(C)OC(=O)NCCc1cn(Cc2ccccc2)c2cc(CN)ccc12]

To a solution of tert-butyl (2-(1-benzyl-6-cyano-1H-indol-3-yl)ethyl)carbamate (445 mg, 1.2 mmol) in ammonia in methanol (7 N, 20 mL), a scoop of Rainey Nickel suspended in water was added. The reaction was charged with hydrogen (60 psi) and stirred at rt for 18 h. After degassing with nitrogen, the mixture was filtered over celite. The filtrate was concentrated to afford the desired product (430 mg, crude) as a yellow foam. ESI MS m/z 380 [M+1]+.

### Step 3. Tert-butyl (2-(1-benzyl-6-((((3-(6-hydroxy-3-oxoisoindolin-1-yl)-1H-indol-2-yl)methyl)amino)methyl)-1H-indol-3-yl)ethyl)carbamate (JRW-2085)

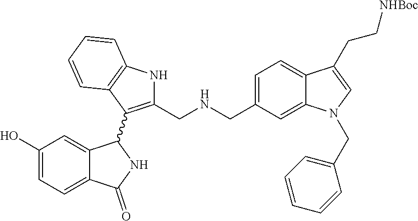 [SMILES: CC(C)(C)OC(=O)NCCc1cn(Cc2ccccc2)c2cc(CNCc3[nH]c4ccccc4c3C3NC(=O)c4ccc(O)cc43)ccc12]

To a suspension of tert-butyl (2-(6-(aminomethyl)-1-benzyl-1H-indol-3-yl)ethyl)carbamate (116 mg, 0.31 mmol) and 3-(6-hydroxy-3-oxoisoindolin-1-yl)-1H-indole-2-carbaldehyde (75 mg, 0.25 mmol) in toluene (10 mL), magnesium sulfate (200 mg) was added. The suspension was heated at 100° C. for 18 h. The reaction was cooled, concentrated, and resuspended in methanol (10 mL). The mixture was cooled to 0° C., and sodium borohydride (29 mg, 0.77 mmol) was added. The reduction was warmed to rt and stirred for 2 h. The reaction was diluted with methanol, celite was added, concentrated, and purified with silica gel chromatography to afford the desired product (78 mg, 46%) as a brown glass. ESI MS m/z 656 [M+1]+.

### Step 4. 3-(2-((((3-(2-aminoethyl)-1-benzyl-1H-indol-6-yl)methyl)amino)methyl)-1H-indol-3-yl)-5-hydroxyisoindolin-1-one (JRW-2091)

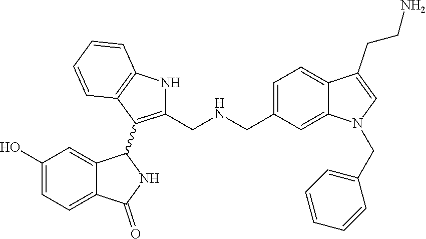 [SMILES: NCCc1cn(Cc2ccccc2)c2cc(CNCc3[nH]c4ccccc4c3C3NC(=O)c4ccc(O)cc43)ccc12]

To a solution of tert-butyl (2-(1-benzyl-6-((((3-(6-hydroxy-3-oxoisoindolin-1-yl)-1H-indol-2-yl)methyl)amino)methyl)-1H-indol-3-yl)ethyl)carbamate (78 mg, 0.12 mmol) in dichloromethane (10 mL), trifluoroacetic acid (1 mL) was added. The reaction stirred at rt for 2 h. The mixture was concentrated to give crude product as a brown solid. ESI MS m/z 556 [M+1]+.

### Step 5. N-(2-(1-benzyl-6-((((3-(6-hydroxy-3-oxoisoindolin-1-yl)-1H-indol-2-yl)methyl)amino)methyl)-1H-indol-3-yl)ethyl)-1-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-3,6,9,12-tetraoxapentadecan-15-amide (JRW-2095)

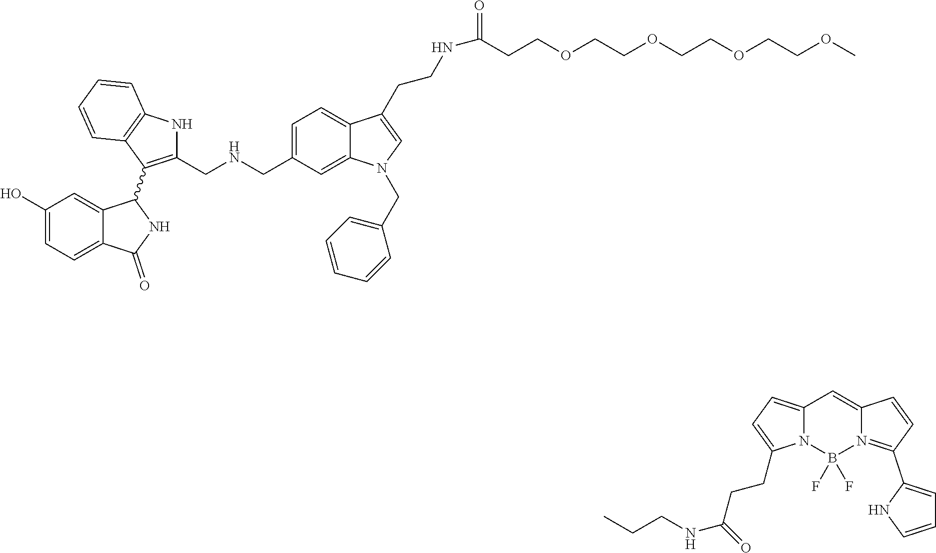

To a solution of 3-(2-((((3-(2-aminoethyl)-1-benzyl-1H-indol-6-yl)methyl)amino)methyl)-1H-indol-3-yl)-5-hydroxyisoindolin-1-one (15 mg, 0.027 mmol) in DMF (5 mL), 2,5-dioxopyrrolidin-1-yl 1-(5,5-difluoro-7-(1H-pyrrol-2-yl)-5H-5λ⁴,6λ⁴-dipyrrolo[1,2-c:2′,1′-f][1,3,2]diazaborinin-3-yl)-3-oxo-7,10,13,16-tetraoxa-4-azanonadecan-19-oate (9 mg, 0.013 mmol) and diisopropylethylamine (14 mg, 0.11 mmol) was added. The reaction was stirred at rt for 30 min. The mixture was diluted with methanol and purified by reverse phase preparative HPLC to afford the desired product (3 mg, 20%) as a purple solid. ESI MS m/z 1132 [M+1]+.

### N-(2-(1-benzyl-6-((((3-(6-hydroxy-3-oxoisoindolin-1-yl)-1H-indol-2-yl)methyl)amino)methyl)-1H-indol-3-yl)ethyl)-3-(5,5-difluoro-7-(1H-pyrrol-2-yl)-5H-5λ⁴,6λ⁴-dipyrrolo[1,2-c:2′,1′-f][1,3,2]diazaborinin-3-yl)propenamide (JRW-2096)

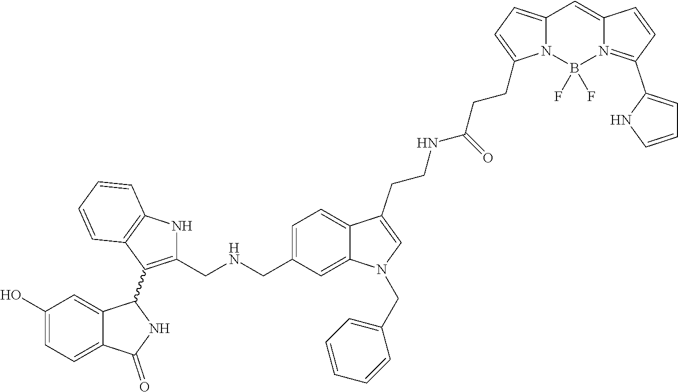

To a solution of 3-(2-((((3-(2-aminoethyl)-1-benzyl-1H-indol-6-yl)methyl)amino)methyl)-1H-indol-3-yl)-5-hydroxyisoindolin-1-one (15 mg, 0.027 mmol) in DMF (5 mL), 2,5-dioxopyrrolidin-1-yl 3-(5,5-difluoro-7-(1H-pyrrol-2-yl)-5H-5λ⁴,6λ⁴-dipyrrolo[1,2-c:2′,1′-f][1,3,2]diazaborinin-3-yl)propanoate (11 mg, 0.027 mmol) and diisopropylethylamine (28 mg, 0.22 mmol) was added. The reaction was stirred at rt for 30 min. The mixture was diluted with methanol and purified by reverse phase preparative HPLC to afford the desired product (10 mg, 43%) as a purple solid. ESI MS m/z 867 [M+1]+.

### N-(2-(1-benzyl-6-((((3-(6-hydroxy-3-oxoisoindolin-1-yl)-1H-indol-2-yl)methyl)amino)methyl)-1H-indol-3-yl)ethyl)-4-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)butanamide (JRW-2097)

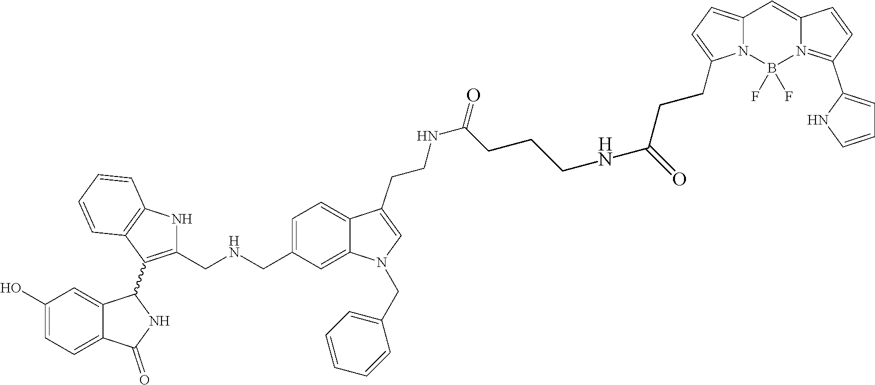

To a solution of 3-(2-((((3-(2-aminoethyl)-1-benzyl-1H-indol-6-yl)methyl)amino)methyl)-1H-indol-3-yl)-5-hydroxyisoindolin-1-one (15 mg, 0.027 mmol) in DMF (5 mL), 2,5-dioxopyrrolidin-1-yl 4-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)butanoate (13 mg, 0.027 mmol) and diisopropylethylamine (28 mg, 0.22 mmol) was added. The reaction was stirred at rt for 30 min. The mixture was diluted with methanol and purified by reverse phase preparative HPLC to afford the desired product (6 mg, 24%) as a purple solid. ESI MS m/z 952 [M+1]+.

### 1-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-N-(4-((6-((((3-(6-hydroxy-3-oxoisoindolin-1-yl)-1H-indol-2-yl)methyl)amino)methyl)-1H-indol-1-yl)methyl)benzyl)-3,6,9,12-tetraoxapentadecan-15-amide (JRW-2111)

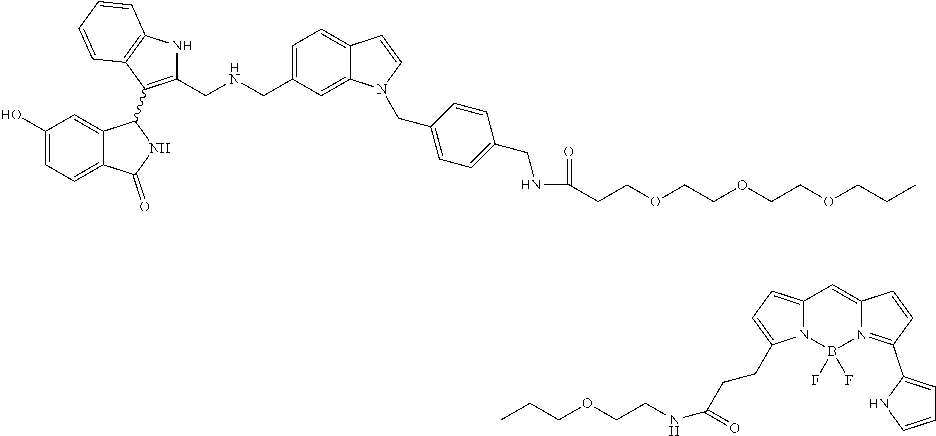

### Step 1. Tert-butyl (4-((6-cyano-1H-indol-1-yl)methyl)benzyl)carbamate (JRW-2105)

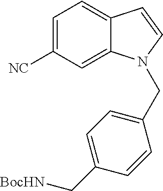 [SMILES: CC(C)(C)OC(=O)NCc1ccc(Cn2ccc3ccc(C#N)cc32)cc1]

To a solution of 1H-indole-6-carbonitrile (500 mg, 3.5 mmol) in DMF (20 mL) chilled with an ice bath, sodium hydride (211 mg, 5.3 mmol, 60%), tert-butyl (4-(chloromethyl)benzyl)carbamate (1.1 g, 4.2 mmol), and tetrabutylammonium iodide (130 mg, 0.35 mmol) was added. The mixture stirred at 0° C. for 30 min and rt for 1 h. The reaction was diluted with ethyl acetate and washed with water. The organic layers were combined, dried with sodium sulfate, filtered, concentrated, and purified with silica gel chromatography to afford the desired product (1.2 g, 94%) as a white solid. ESI MS m/z 362 [M+1]+.

### Step 2. Tert-butyl (4-((6-(aminomethyl)-1H-indol-1-yl)methyl)benzyl)carbamate (JRW-2106)

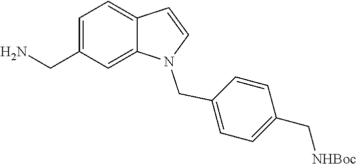 [SMILES: CC(C)(C)OC(=O)NCc1ccc(Cn2ccc3ccc(CN)cc32)cc1]

To a solution of tert-butyl (4-((6-cyano-1H-indol-1-yl)methyl)benzyl)carbamate (500 mg, 1.4 mmol) in ammonia in methanol (7 N, 20 mL), a scoop of Rainey Nickel suspended in water was added. The reaction was charged with hydrogen (60 psi) and stirred at rt for 18 h. After degassing with nitrogen, the mixture was filtered over celite. The filtrate was concentrated to afford the crude product as a yellow solid. ESI MS m/z 366 [M+1]+.

### Step 3. tert-butyl (4-((6-((((3-(6-hydroxy-3-oxoisoindolin-1-yl)-1H-indol-2-yl)methyl)amino)methyl)-1H-indol-1-yl)methyl)benzyl)carbamate (JRW-2109)

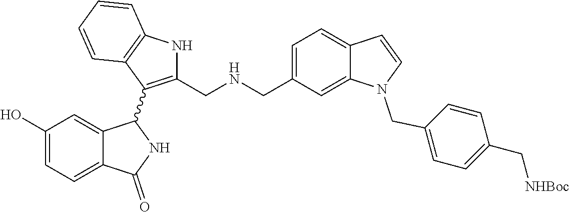 [SMILES: CC(C)(C)OC(=O)NCc1ccc(Cn2ccc3ccc(CNCc4[nH]c5ccccc5c4C4NC(=O)c5ccc(O)cc54)cc32)cc1]

To a suspension of tert-butyl (4-((6-(aminomethyl)-1H-indol-1-yl)methyl)benzyl)carbamate (275 mg, 0.75 mmol) and 3-(6-hydroxy-3-oxoisoindolin-1-yl)-1H-indole-2-carbaldehyde (110 mg, 0.37 mmol) in toluene (10 mL), magnesium sulfate (200 mg) was added. The suspension was heated at 100° C. for 18 h. The reaction was cooled, concentrated, and resuspended in methanol (10 mL). The mixture was cooled to 0° C., and sodium borohydride (43 mg, 1.1 mmol) was added. The reduction was warmed to rt and stirred for 30 min. The reaction was diluted with methanol, celite was added, concentrated, and purified with silica gel chromatography to afford the desired product (45 mg, 18%) as a white solid. ESI MS m/z 642 [M+1]+.

### Step 4.3-(2-((((1-(4-(aminomethyl)benzyl)-1H-indol-6-yl)methyl)amino)methyl)-1H-indol-3-yl)-5-hydroxyisoindolin-1-one (JRW-2110)

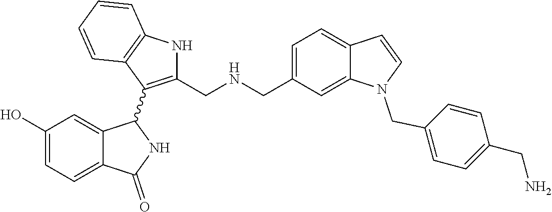 [SMILES: NCc1ccc(Cn2ccc3ccc(CNCc4[nH]c5ccccc5c4C4NC(=O)c5ccc(O)cc54)cc32)cc1]

To a solution of tert-butyl (4-((6-((((3-(6-hydroxy-3-oxoisoindolin-1-yl)-1H-indol-2-yl)methyl)amino)methyl)-1H-indol-1-yl)methyl)benzyl)carbamate (45 mg, 0.074 mmol) in dichloromethane (10 mL), trifluoroacetic acid (1 mL) was added. The reaction stirred at rt for 30 min. The mixture was concentrated to give crude product as a light brown solid. ESI MS m/z 542 [M+1]+.

### Step 5. 1-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-N-(4-((6-((((3-(6-hydroxy-3-oxoisoindolin-1-yl)-1H-indol-2-yl)methyl)amino)methyl)-1H-indol-1-yl)methyl)benzyl)-3,6,9,12-tetraoxapentadecan-15-amide (JRW-2111)

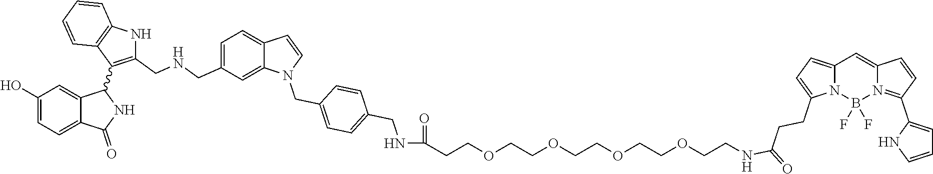

To a solution of 3-(2-((((1-(4-(aminomethyl)benzyl)-1H-indol-6-yl)methyl)amino)methyl)-1H-indol-3-yl)-5-hydroxyisoindolin-1-one (15 mg, 0.027 mmol) in DMF (5 mL), 2,5-dioxopyrrolidin-1-yl 1-(5,5-difluoro-7-(1H-pyrrol-2-yl)-5H-5λ⁴,6λ⁴-dipyrrolo[1,2-c:2′,1′-f][1,3,2]diazaborinin-3-yl)-3-oxo-7,10,13,16-tetraoxa-4-azanonadecan-19-oate (9 mg, 0.013 mmol) and diisopropylethylamine (28 mg, 0.22 mmol) was added. The reaction was stirred at rt for 30 min. The mixture was diluted with methanol and purified by reverse phase preparative HPLC to afford the desired product (5 mg, 16%) as a purple solid. ESI MS m/z 1100 [M+1]+.

### 4-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-N-(4-((6-((((3-(6-hydroxy-3-oxoisoindolin-1-yl)-1H-indol-2-yl)methyl)amino)methyl)-1H-indol-1-yl)methyl)benzyl)butanamide (JRW-2113)

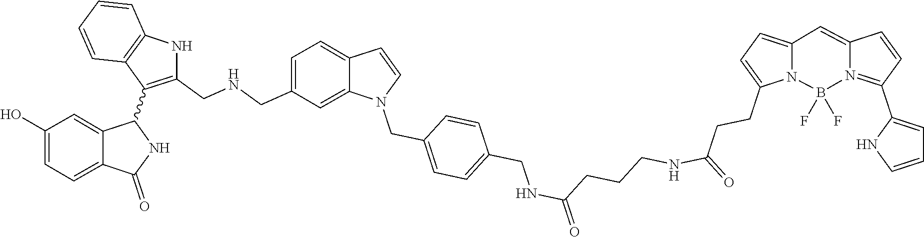

To a solution of 3-(2-((((1-(4-(aminomethyl)benzyl)-1H-indol-6-yl)methyl)amino)methyl)-1H-indol-3-yl)-5-hydroxyisoindolin-1-one (15 mg, 0.027 mmol) in DMF (5 mL), 2,5-dioxopyrrolidin-1-yl 4-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)butanoate (7 mg, 0.013 mmol) and diisopropylethylamine (28 mg, 0.22 mmol) was added. The reaction was stirred at rt for 30 min. The mixture was diluted with methanol and purified by reverse phase preparative HPLC to afford the desired product (8 mg, 30%) as a purple solid. ESI MS m/z 938 [M+1]+.

### 3-(5,5-difluoro-7-(1H-pyrrol-2-yl)-5H-5λ⁴,6λ⁴-dipyrrolo[1,2-c:2′,1′-f][1,3,2]diazaborinin-3-yl)-N-(4-((6-((((3-(6-hydroxy-3-oxoisoindolin-1-yl)-1H-indol-2-yl)methyl)amino)methyl)-1H-indol-1-yl)methyl)benzyl)propenamide (JRW-2114)

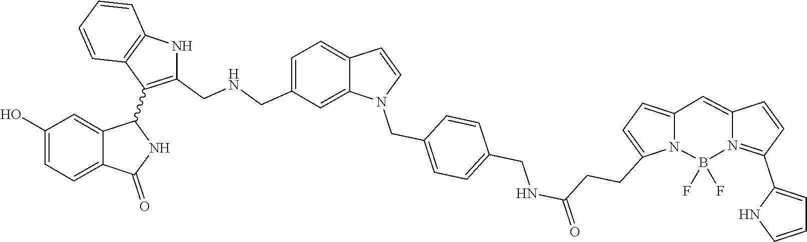

To a solution of 3-(2-((((1-(4-(aminomethyl)benzyl)-1H-indol-6-yl)methyl)amino)methyl)-1H-indol-3-yl)-5-hydroxyisoindolin-1-one (15 mg, 0.027 mmol) in DMF (5 mL), 2,5-dioxopyrrolidin-1-yl 3-(5,5-difluoro-7-(1H-pyrrol-2-yl)-5H-5λ⁴,6λ⁴-dipyrrolo[1,2-c:2′,1′-f][1,3,2]diazaborinin-3-yl)propanoate (6 mg, 0.013 mmol) and diisopropylethylamine (28 mg, 0.22 mmol) was added. The reaction was stirred at rt for 30 min. The mixture was diluted with methanol and purified by reverse phase preparative HPLC to afford the desired product (6 mg, 26%) as a purple solid. ESI MS m/z 853 [M+1]+.

### (S)-1-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-N-(2-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)ethyl)-3,6,9,12-tetraoxapentadecan-15-amide (JRW-2143)

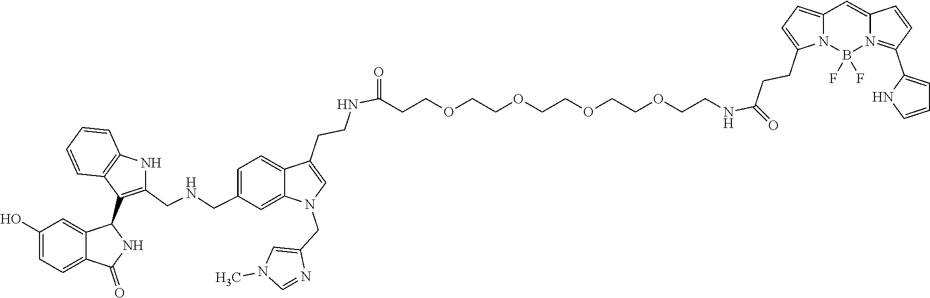

### Step 1. (S)-3-(2-((((3-(2-aminoethyl)-1-((1-methyl-1H-imidazo-4-yl)methyl-1H-indol-6-yl)methyl)amino)methyl)-1H-indol-3-yl)-5-hydroxyisoindolin-1-one (JRW-2141)

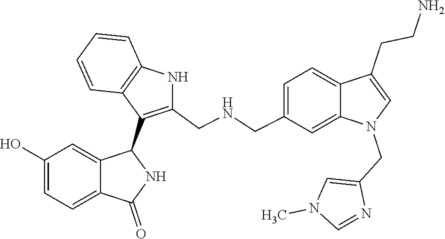 [SMILES: Cn1cnc(Cn2cc(CCN)c3ccc(CNCc4[nH]c5ccccc5c4[C@H]4NC(=O)c5ccc(O)cc54)cc32)c1]

To a solution of tert-butyl (S)-(2-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)ethyl)carbamate (14 mg, 0.021 mmol) in dichloromethane (10 mL), trifluoroacetic acid (1 mL) was added. The reaction stirred at rt for 4 h. The mixture was concentrated to give crude product as a light brown oil. ESI MS m/z 560 [M+1]+.

### Step 2. (S)-1-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-N-(2-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)ethyl)-3,6,9,12-tetraoxapentadecan-15-amide (JRW-2143)

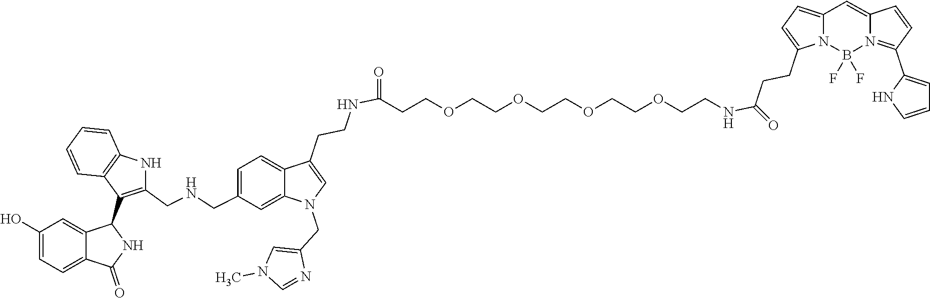

To a solution of (S)-3-(2-((((3-(2-aminoethyl)-1-((1-methyl-1H-imidazol-4-yl)methyl)-1H-indol-6-yl)methyl)amino)methyl)-1H-indol-3-yl)-5-hydroxyisoindolin-1-one (10 mg, 0.017 mmol) in DMF (5 mL), 2,5-dioxopyrrolidin-1-yl 1-(5,5-difluoro-7-(1H-pyrrol-2-yl)-5H-5λ⁴,6λ⁴-dipyrrolo[1,2-c:2′,1′-f][1,3,2]diazaborinin-3-yl)-3-oxo-7,10,13,16-tetraoxa-4-azanonadecan-19-oate (12 mg, 0.017 mmol) and diisopropylethylamine (18 mg, 0.14 mmol) was added. The reaction was stirred at rt for 30 min. The mixture was diluted with methanol and purified by reverse phase preparative HPLC to afford the desired product (II mg, 55%) as a purple solid. ESI MS m/z 1118 [M+1]+.

### (R)-1-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-N-(2-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)ethyl)-3,6,9,12-tetraoxapentadecan-15-amide (JRW-2144)

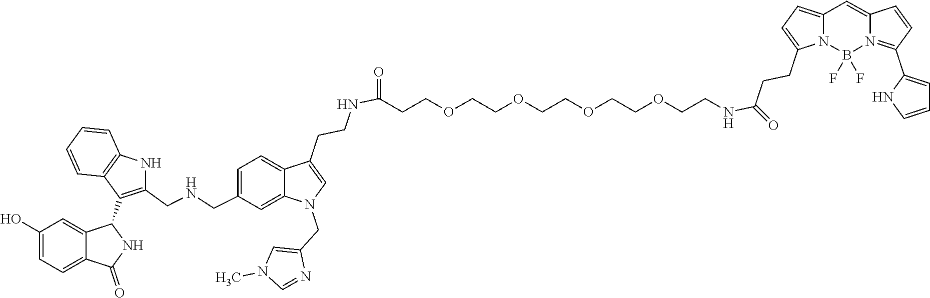

### Step 1. (R)-3-(2-((((3-(2-aminoethyl)-1-((1-methyl-1H-imidazol-4-yl)methyl)-1H-indol-6-yl)methyl)amino)methyl)-1H-indol-3-yl)-5-hydroxyisoindolin-1-one JRW 2142)

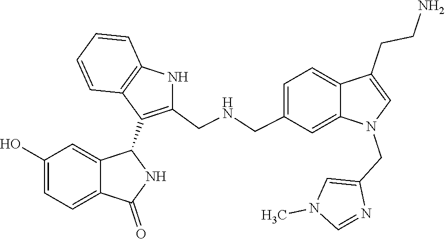 [SMILES: Cn1cnc(Cn2cc(CCN)c3ccc(CNCc4[nH]c5ccccc5c4[C@@H]4NC(=O)c5ccc(O)cc54)cc32)c1]

To a solution of tert-butyl (R)-(2-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)ethyl)carbamate (10 mg, 0.015 mmol) in dichloromethane (10 mL), trifluoroacetic acid (1 mL) was added. The reaction stirred at rt for 4 h. The mixture was concentrated to give crude product as a light brown oil. ESI MS m/z 560 [M+1]+.

### Step 2. (R)-1-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-N-(2-(6-((((3-(6-hydroxy-3-oxoisoindolin-1-yl)-1H-indol-2-yl)methyl)amino)methyl)-1-((1-methyl-1H-imidazol-4-yl)methyl)-1H-indol-3-yl)ethyl)-3,6,9,12-tetraoxapentadecan-15-amide (JRW-2144)

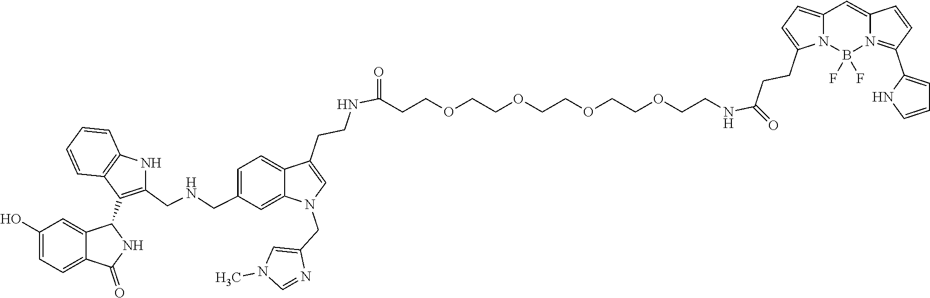

To a solution of (R)-3-(2-((((3-(2-aminoethyl)-1-((1-methyl-1H-imidazol-4-yl)methyl)-1H-indol-6-yl)methyl)amino)methyl)-1H-indol-3-yl)-5-hydroxyisoindolin-1-one (10 mg, 0.017 mmol) in DMF (5 mL), 2,5-dioxopyrrolidin-1-yl 1-(5,5-difluoro-7-(1H-pyrrol-2-yl)-5H-5λ⁴,6λ⁴-dipyrrolo[1,2-c:2′,1′-f][1,3,2]diazaborinin-3-yl)-3-oxo-7,10,13,16-tetraoxa-4-azanonadecan-19-oate (12 mg, 0.017 mmol) and diisopropylethylamine (18 mg, 0.14 mmol) was added. The reaction was stirred at rt for 30 min. The mixture was diluted with methanol and purified by reverse phase preparative HPLC to afford the desired product (10 mg, 50%) as a purple solid. ESI MS m/z 1118 [M+1]+.

### 3-(5,5-difluoro-7-(1H-pyrrol-2-yl)-5H-5λ⁴,6λ⁴-dipyrrolo[1,2-c:2′,1′-f][1,3,2]diazaborinin-3-yl)-N-(3-((6-((((3-(6-hydroxy-3-oxoisoindolin-1-yl)-1H-indol-2-yl)methyl)amino)methyl)-1H-indol-1-yl)methyl)benzyl)propenamide (JRW-2191)

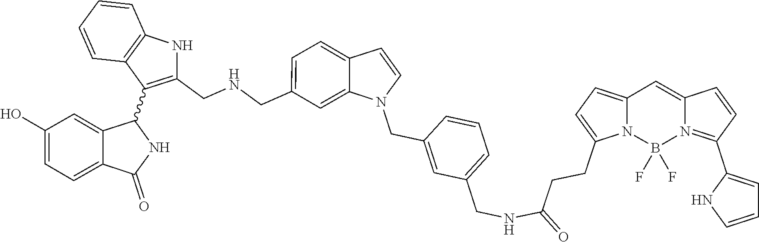

### Step 1. tert-butyl (3-((6-cyano-1H-indol-1-yl)methyl)benzyl)carbamate (JRW-2181)

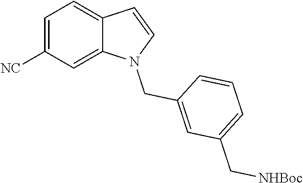 [SMILES: CC(C)(C)OC(=O)NCc1cccc(Cn2ccc3ccc(C#N)cc32)c1]

To a solution of 1H-indole-6-carbonitrile (450 mg, 3.2 mmol) in DMF (20 mL) chilled with an ice bath, sodium hydride (190 mg, 4.8 mmol, 60%), tert-butyl (4-(chloromethyl)benzyl)carbamate (0.81 g, 3.2 mmol), and tetrabutylammonium iodide (117 mg, 0.32 mmol) was added. The mixture stirred at 0° C. for 15 min and rt for 2 h. The reaction was diluted with ethyl acetate and washed with water. The organic layers were combined, dried with sodium sulfate, filtered, concentrated, and purified with silica gel chromatography to afford the desired product (0.90 g, 78%) as an oil. ESI MS m/z 362 [M+1]+.

### Step 2. tert-butyl (3-((6-(aminomethyl)-1H-indol-1-yl)methyl)benzyl)carbamate (JRW-2182)

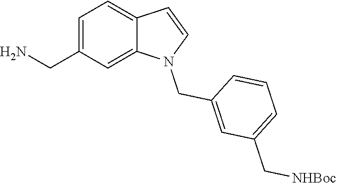 [SMILES: CC(C)(C)OC(=O)NCc1cccc(Cn2ccc3ccc(CN)cc32)c1]

To a solution of tert-butyl (3-((6-cyano-1H-indol-1-yl)methyl)benzyl)carbamate (0.90 g, 2.5 mmol) in ammonia in methanol (7 N, 20 mL), a scoop of Rainey Nickel suspended in water was added. The reaction was charged with hydrogen (60 psi) and stirred at rt for 18 h. After degassing with nitrogen, the mixture was filtered over celite. The filtrate was concentrated and purified with silica gel chromatography to afford the desired product (0.59 g, 64%) as a foam. ESI MS m/z 366 [M+1]+.

### Step 3. tert-butyl (3-((6-((((3-(6-hydroxy-3-oxoisoindolin-1-yl)-1H-indol-2-yl)methyl)amino)methyl)-1H-indol-1-yl)methyl)benzyl)carbamate (JRW-2186)

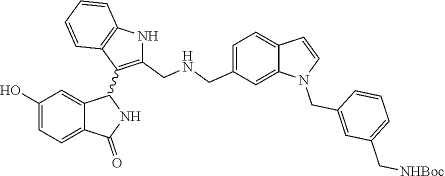 [SMILES: CC(C)(C)OC(=O)NCc1cccc(Cn2ccc3ccc(CNCc4[nH]c5ccccc5c4C4NC(=O)c5ccc(O)cc54)cc32)c1]

To a suspension of tert-butyl (3-((6-(aminomethyl)-1H-indol-1-yl)methyl)benzyl)carbamate (150 mg, 0.41 mmol) and 3-(6-hydroxy-3-oxoisoindolin-1-yl)-1H-indole-2-carbaldehyde (120 mg, 0.41 mmol) in toluene (10 mL), magnesium sulfate (200 mg) was added. The suspension was heated at 100° C. for 18 h. The reaction was cooled, concentrated, and resuspended in methanol (10 mL). The mixture was cooled to 0° C., and sodium borohydride (46 mg, 1.2 mmol) was added. The reduction was warmed to rt and stirred for 30 min. The reaction was diluted with methanol, celite was added, concentrated, and purified with silica gel chromatography to afford the desired product (27 mg, 10%) as a white solid. ESI MS m/z 642 [M+1]+.

### Step 4. 3-(2-((((1-(3-(aminomethyl)benzyl)-1H-indol-6-yl)methyl)amino)methyl)-1H-indol-3-yl)-5-hydroxyisoindolin-1-one (JRW-2190)

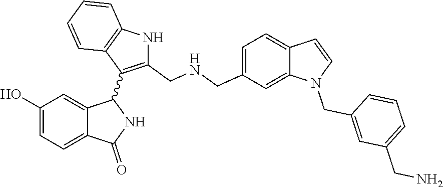 [SMILES: NCc1cccc(Cn2ccc3ccc(CNCc4[nH]c5ccccc5c4C4NC(=O)c5ccc(O)cc54)cc32)c1]

To a solution of tert-butyl (3-((6-((((3-(6-hydroxy-3-oxoisoindolin-1-yl)-1H-indol-2-yl)methyl)amino)methyl)-1H-indol-1-yl)methyl)benzyl)carbamate (27 mg, 0.042 mmol) in dichloromethane (10 mL), trifluoroacetic acid (1 mL) was added. The reaction stirred at rt for 30 min. The mixture was concentrated to give crude product as a light brown solid. ESI MS m/z 542 [M+1]+.

### Step 5. 3-(5,5-difluoro-7-(1H-pyrrol-2-yl)-5H-5λ⁴,6λ⁴-dipyrrolo[1,2-c:2′,1′-f][1,3,2]diazaborinin-3-yl)-N-(3-((6-((((3-(6-hydroxy-3-oxoisoindolin-1-yl)-1H-indol-2-yl)methyl)amino)methyl)-1H-indol-1-yl)methyl)benzyl)propenamide (JRW-2191)

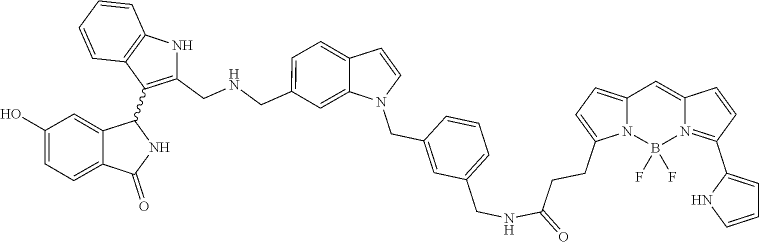

To a solution of 3-(2-((((1-(3-(aminomethyl)benzyl)-1H-indol-6-yl)methyl)amino)methyl)-1H-indol-3-yl)-5-hydroxyisoindolin-1-one (15 mg, 0.027 mmol) in DMF (5 mL), 2,5-dioxopyrrolidin-1-yl 3-(5,5-difluoro-7-(1H-pyrrol-2-yl)-5H-5λ⁴,6λ⁴-dipyrrolo[1,2-c:2′,1′-f][1,3,2]diazaborinin-3-yl)propanoate (11 mg, 0.027 mmol) and diisopropylethylamine (28 mg, 0.22 mmol) was added. The reaction was stirred at rt for 30 min. The mixture was diluted with methanol and purified by reverse phase preparative HPLC to afford the desired product (5 mg, 21%) as a purple solid. ESI MS m/z 853 [M+1]+.

### 1-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-N-(3-((6-((((3-(6-hydroxy-3-oxoisoindolin-1-yl)-1H-indol-2-yl)methyl)amino)methyl)-1H-indol-1-yl)methyl)benzyl)-3,6,9,12-tetraoxapentadecan-15-amide (JRW-2192)

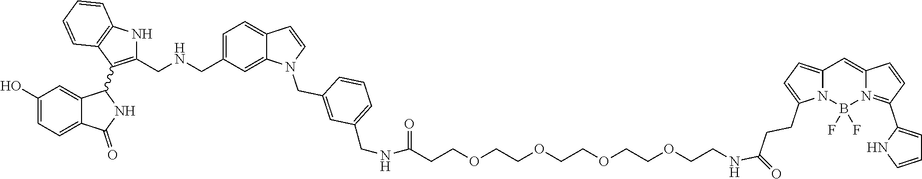

To a solution of 3-(2-((((1-(3-(aminomethyl)benzyl)-1H-indol-6-yl)methyl)amino)methyl)-1H-indol-3-yl)-5-hydroxyisoindolin-1-one (prepared as described above) (15 mg, 0.027 mmol) in DMF (5 mL), 2,5-dioxopyrrolidin-1-yl 1-(5,5-difluoro-7-(1H-pyrrol-2-yl)-5H-5λ⁴,6λ⁴-dipyrrolo[1,2-c:2′,1′-f][1,3,2]diazaborinin-3-yl)-3-oxo-7,10,13,16-tetraoxa-4-azanonadecan-19-oate (9 mg, 0.013 mmol) and diisopropylethylamine (28 mg, 0.22 mmol) was added. The reaction was stirred at rt for 30 min. The mixture was diluted with methanol and purified by reverse phase preparative HPLC to afford the desired product (6 mg, 20%) as a purple solid. ESI MS m/z 1100 [M+1]+.

### 3-(5,5-difluoro-7-(1H-pyrrol-2-yl)-5H-5λ⁴,6λ⁴-dipyrrolo[1,2-c:2′,1′-f][1,3,2]diazaborinin-3-yl)-N-(3-(4-((6-((((3-(6-hydroxy-3-oxoisoindolin-1-yl)-1H-indol-2-yl)methyl)amino)methyl)-1H-indol-1-yl)methyl)-1H-imidazol-1-yl)propyl)propenamide (JRW-2218)

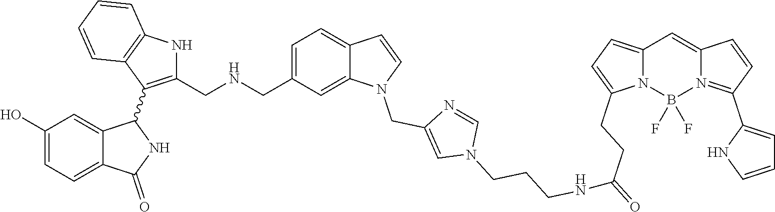

### Step 1. 1-((1H-imidazol-4-yl)methyl)-1H-indole-6-carbonitrile (JRW-2189)

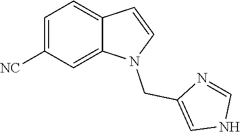 [SMILES: N#Cc1ccc2ccn(Cc3c[nH]cn3)c2c1]

To a solution of 1H-indole-6-carbonitrile (600 mg, 4.2 mmol) in DMF (20 mL) chilled with an ice bath, sodium hydride (253 mg, 6.3 mmol, 60%), tert-butyl 4-(chloromethyl)-1H-imidazole-1-carboxylate (0.91 g, 4.2 mmol), and tetrabutylammonium iodide (155 mg, 0.42 mmol) was added. The mixture stirred at 0° C. for 15 min and rt for 18 h. The reaction was diluted with water, the pH was adjusted to 12 with sodium hydroxide, and extracted with chloroform/isopropanol (3:1). The organic layers were combined, dried with sodium sulfate, filtered, concentrated, and purified with silica gel chromatography to afford the desired product (130 mg, 9%) as a light yellow solid. ESI MS m/z 223 [M+1]+.

### Step 2. tert-butyl (3-(4-((6-cyano-1H-indol-1-yl)methyl)-1H-imidazol-1-yl)propyl)carbamate (JRW-2211)

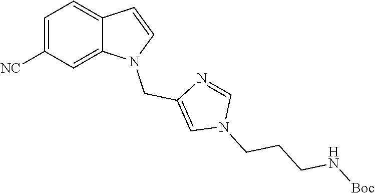 [SMILES: CC(C)(C)OC(=O)NCCCn1cnc(Cn2ccc3ccc(C#N)cc32)c1]

To a solution of 1-((1H-imidazol-4-yl)methyl)-1H-indole-6-carbonitrile (110 mg, 0.49 mmol) in DMF (20 mL) chilled with an ice bath, sodium hydride (30 mg, 0.74 mmol, 60%) and tert-butyl (3-bromopropyl)carbamate (177 mg, 0.74 mmol) was added. The mixture stirred at 0° C. for 15 min and rt for 2 h. The reaction was diluted with ethyl acetate and washed with water. The organic layers were combined, dried with sodium sulfate, filtered, concentrated, and purified with silica gel chromatography to afford the desired product (112 mg, 59%) as an orange oil. ESI MS m/z 380 [M+1]+.

### Step 3. tert-butyl (3-(4-((6-(aminomethyl)-1H-indol-1-yl)methyl)-1H-imidazol-1-yl)propyl)carbamate (JRW-2213)

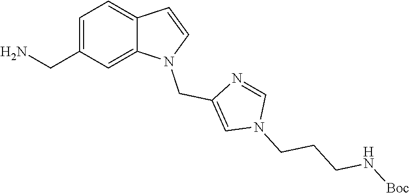 [SMILES: CC(C)(C)OC(=O)NCCCn1cnc(Cn2ccc3ccc(CN)cc32)c1]

To a solution of tert-butyl (3-(4-((6-cyano-1H-indol-1-yl)methyl)-1H-imidazol-1-yl)propyl)carbamate (100 mg, 0.26 mmol) in ammonia in methanol (7 N, 20 mL), a scoop of Rainey Nickel suspended in water was added. The reaction was charged with hydrogen (60 psi) and stirred at rt for 18 h. After degassing with nitrogen, the mixture was filtered over celite. The filtrate was concentrated to afford the crude product (110 mg) as a semisolid. ESI MS m/z 384 [M+1]+.

### Step 4. tert-butyl (3-(4-((6-((((3-(6-hydroxy-3-oxoisoindolin-1-yl)-1H-indol-2-yl)methyl)amino)methyl)-1H-indol-1-yl)methyl)-1H-imidazol-1-yl)propyl)carbamate (JRW-2214)

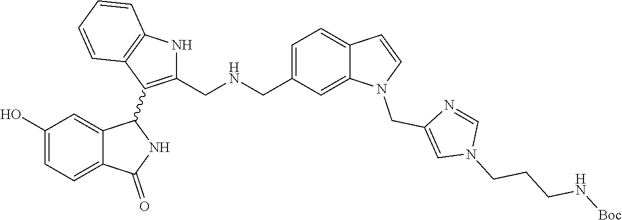 [SMILES: CC(C)(C)OC(=O)NCCCn1cnc(Cn2ccc3ccc(CNCc4[nH]c5ccccc5c4C4NC(=O)c5ccc(O)cc54)cc32)c1]

To a suspension of tert-butyl (3-(4-((6-(aminomethyl)-1H-indol-1-yl)methyl)-1H-imidazol-1-yl)propyl)carbamate (98 mg, 0.26 mmol) in THF (10 mL), 3-(6-hydroxy-3-oxoisoindolin-1-yl)-1H-indole-2-carbaldehyde (75 mg, 0.26 mmol) was added. The reaction stirred at rt for 1 h. Sodium triacetoxyborohydride (272 mg, 1.3 mmol) was added and stirred at rt for 18 h. The reaction was diluted with methanol, celite was added, concentrated, and purified with silica gel chromatography to afford the desired product (125 mg, 73%) as an orange oil. ESI MS m/z 660 [M+1]+.

### Step 5. 3-(2-((((1-((1-(3-aminopropyl)-1H-imidazol-4-yl)methyl)-1H-indol-6-yl)methyl)amino)methyl)-1H-indol-3-yl)-5-hydroxyisoindolin-1-one (JRW-2216)

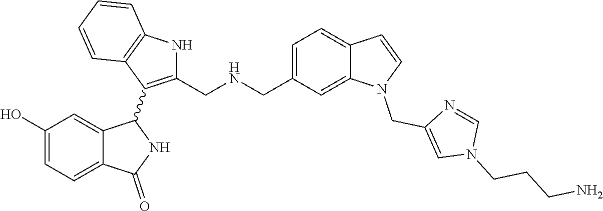 [SMILES: NCCCn1cnc(Cn2ccc3ccc(CNCc4[nH]c5ccccc5c4C4NC(=O)c5ccc(O)cc54)cc32)c1]

To a solution of tert-butyl (3-(4-((6-((((3-(6-hydroxy-3-oxoisoindolin-1-yl)-1H-indol-2-yl)methyl)amino)methyl)-1H-indol-1-yl)methyl)-1H-imidazol-1-yl)propyl)carbamate (125 mg, 0.19 mmol) in dichloromethane (10 mL), trifluoroacetic acid (1 mL) was added. The reaction stirred at rt for 6 h. The mixture was concentrated to give crude product as a brown solid. ESI MS m/z 560 [M+1]+.

### Step 6. 3-(5,5-difluoro-7-(1H-pyrrol-2-yl)-5H-5λ⁴,6λ⁴-dipyrrolo[1,2-c:2′,1′-f][1,3,2]diazaborinin-3-yl)-N-(3-(4-((6-((((3-(6-hydroxy-3-oxoisoindolin-1-yl)-1H-indol-2-yl)methyl)amino)methyl)-1H-indol-1-yl)methyl)-1H-imidazol-1-yl)propyl)propenamide (JRW-2218)

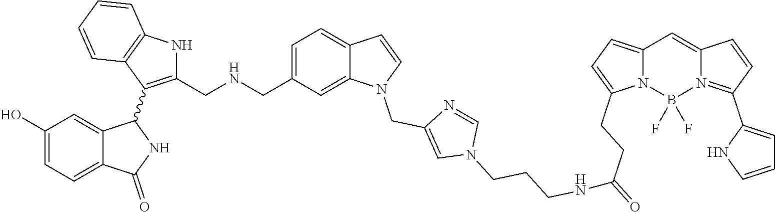

To a solution of 3-(2-((((1-((1-(3-aminopropyl)-1H-imidazol-4-yl)methyl)-1H-indol-6-yl)methyl)amino)methyl)-1H-indol-3-yl)-5-hydroxyisoindolin-1-one (prepared as described above) (15 mg, 0.027 mmol) in DMF (5 mL), 2,5-dioxopyrrolidin-1-yl 3-(5,5-difluoro-7-(1H-pyrrol-2-yl)-5H-5λ⁴,6λ⁴-dipyrrolo[1,2-c:2′,1′-f][1,3,2]diazaborinin-3-yl)propanoate (11 mg, 0.027 mmol) and diisopropylethylamine (28 mg, 0.22 mmol) was added. The reaction was stirred at rt for 30 min. The mixture was diluted with methanol and purified by reverse phase preparative HPLC to afford the desired product (24 mg, quant) as a purple solid. ESI MS m/z 871 [M+1]+.

### 4-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-N-(3-(4-((6-((((3-(6-hydroxy-3-oxoisoindolin-1-yl)-1H-indol-2-yl)methyl)amino)methyl)-1H-indol-1-yl)methyl)-1H-imidazol-1-yl)propyl)butanamide (JRW-2219)

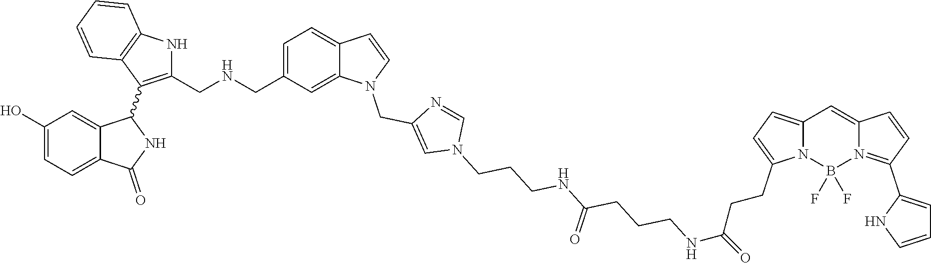

To a solution of 3-(2-((((1-((1-(3-aminopropyl)-1H-imidazol-4-yl)methyl)-1H-indol-6-yl)methyl)amino)methyl)-1H-indol-3-yl)-5-hydroxyisoindolin-1-one (prepared as described above) (15 mg, 0.027 mmol) in DMF (5 mL), 2,5-dioxopyrrolidin-1-yl 4-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)butanoate (13 mg, 0.027 mmol) and diisopropylethylamine (28 mg, 0.22 mmol) was added. The reaction was stirred at rt for 30 min. The mixture was diluted with methanol and purified by reverse phase preparative HPLC to afford the desired product (12 mg, 48%) as a purple solid. ESI MS m/z 956 [M+1]+.

### 1-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-N-(3-(4-((6-((((3-(6-hydroxy-3-oxoisoindolin-1-yl)-1H-indol-2-yl)methyl)amino)methyl)-1H-indol-1-yl)methyl)-1H-imidazol-1-yl)propyl)-3,6,9,12-tetraoxapentadecan-15-amide (JRW-2220)

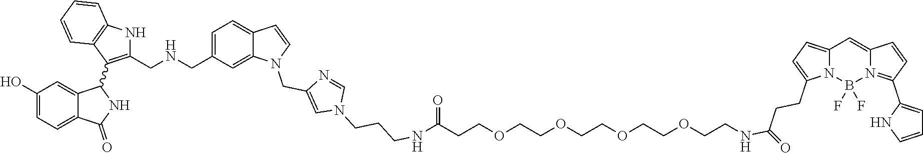

To a solution of 3-(2-((((1-((1-(3-aminopropyl)-1H-imidazol-4-yl)methyl)-1H-indol-6-yl)methyl)amino)methyl)-1H-indol-3-yl)-5-hydroxyisoindolin-1-one (prepared as described above) (15 mg, 0.027 mmol) in DMF (5 mL), 2,5-dioxopyrrolidin-1-yl 1-(5,5-difluoro-7-(1H-pyrrol-2-yl)-5H-5λ⁴,6λ⁴-dipyrrolo[1,2-c:2′,1′-f][1,3,2]diazaborinin-3-yl)-3-oxo-7,10,13,16-tetraoxa-4-azanonadecan-19-oate (9 mg, 0.013 mmol) and diisopropylethylamine (28 mg, 0.22 mmol) was added. The reaction was stirred at rt for 30 min. The mixture was diluted with methanol and purified by reverse phase preparative HPLC to afford the desired product (19 mg, 63%) as a purple solid. ESI MS m/z 1118 [M+1]+.

### N-(15-(5,5-difluoro-7-(1H-pyrrol-2-yl)-5H-5λ⁴,6λ⁴-dipyrrolo[1,2-c:2′,1′-f][1,3,2]diazaborinin-3-yl)-13-oxo-3,6,9-trioxa-12-azapentadecyl)-5-(2-((6-((((3-(6-hydroxy-3-oxoisoindolin-1-yl)-1H-indol-2-yl)methyl)amino)methyl)-1H-indol-1-yl)methyl)-1H-imidazol-1-yl)pentanamide (JRW-2310)

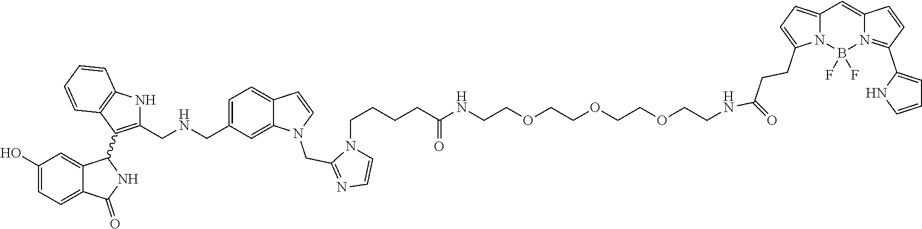

### Step 1. Methyl 5-(2-(hydroxymethyl)-1H-imidazol-1-yl)pentanoate (JRW-2271)

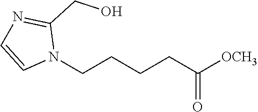 [SMILES: COC(=O)CCCCn1ccnc1CO]

To a solution of methyl 5-(2-formyl-1H-imidazol-1-yl)pentanoate (0.92 g, 4.4 mmol) in methanol/tetrahydrofuran (1:1, 50 mL) chilled with an ice bath, sodium borohydride (200 mg, 5.3 mmol) was added, and the mixture stirred for 30 min. The reaction was quenched with HCl (3 mL, 2M), and then the pH was adjusted to 8. The mixture was concentrated with celite and purified with silica gel chromatography to afford the desired product (0.53 g, 57%) as a colorless oil. ESI MS m/z 213 [M+1]+.

### Step 2. methyl 5-(2-(chloromethyl)-1H-imidazol-1-yl)pentanoate (JRW-2279)

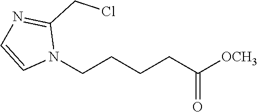 [SMILES: COC(=O)CCCCn1ccnc1CCl]

To a solution of methyl 5-(2-(hydroxymethyl)-1H-imidazol-1-yl)pentanoate (0.53 g, 2.5 mmol) in chloroform, thionyl chloride (3.0 g, 25 mmol) was added. The mixture stirred for 1 h at rt and heated to 75° C. for 1 h. The reaction was concentrated, and the residue was suspended in ether. Filtration afforded the crude product (0.64 g) as a white solid. ESI MS m/z 231 [M+1]+.

### Step 3. methyl 5-(2-((6-cyano-1H-indol-1-yl)methyl)-1H-imidazol-1-yl)pentanoate (JRW-2281)

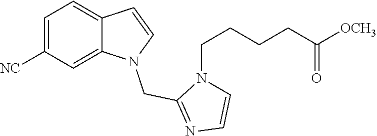 [SMILES: COC(=O)CCCCn1ccnc1Cn1ccc2ccc(C#N)cc21]

To a solution of 1H-indole-6-carbonitrile (200 mg, 1.4 mmol) in DMF (20 mL) chilled with an ice bath, sodium hydride (84 mg, 2.1 mmol, 60%), methyl 5-(2-(chloromethyl)-1H-imidazol-1-yl)pentanoate (0.38 g, 1.4 mmol), and tetrabutylammonium iodide (52 mg, 0.14 mmol) was added. The mixture stirred at 0° C. for 15 min and rt for 2 h. The reaction was diluted with ethyl acetate and washed with water. The organic layers were combined, dried with sodium sulfate, filtered, concentrated, and purified with silica gel chromatography to afford the desired product (370 mg, 78%) as an orange oil. ESI MS m/z 337 [M+1]+.

### Step 4. 5-(2-((6-cyano-1H-indol-1-yl)methyl)-1H-imidazol-1-yl)pentanoic acid (JRW-2284)

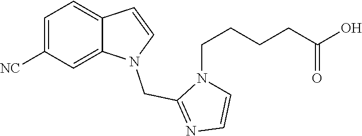 [SMILES: N#Cc1ccc2ccn(Cc3nccn3CCCCC(=O)O)c2c1]

To a solution of methyl 5-(2-((6-cyano-1H-indol-1-yl)methyl)-1H-imidazol-1-yl)pentanoate (0.37 g, 1.1 mmol) in dioxane (10 mL), lithium hydroxide (0.13 g, 5.5 mmol) and water (1 mL) was added. The reaction was heated to 40° C. for 2 h. The mixture diluted with water, the pH was adjusted to 3 with HCl, and extracted with CHCl₃/IPA 3:1. Concentration of the organic layer afforded the crude product (0.30 g) as a white solid. ESI MS m/z 323 [M+1]+.

### Step 5. tert-butyl (17-(2-((6-cyano-1H-indol-1-yl)methyl)-1H-imidazol-1-yl)-13-oxo-3,6,9-trioxa-12-azaheptadecyl)carbamate (JRW-2286)

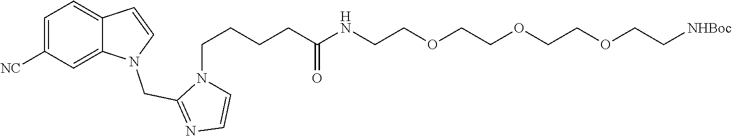 [SMILES: CC(C)(C)OC(=O)NCCOCCOCCOCCNC(=O)CCCCn1ccnc1Cn1ccc2ccc(C#N)cc21]

To a solution of 5-(2-((6-cyano-1H-indol-1-yl)methyl)-1H-imidazol-1-yl)pentanoic acid (0.30 g, 0.93 mmol) in DMF (10 mL), tert-butyl (2-(2-(2-(2-aminoethoxy)ethoxy)ethoxy)ethyl)carbamate (0.33 g, 1.1 mmol), hydroxybenzotriazole (0.28 g, 1.9 mmol), 1-Ethyl-3-(3′-dimethylaminopropyl)carbodiimide, HCl (0.36 g, 1.9 mmol), and diisopropylamine (0.36 g, 2.8 mmol) was added. The reaction was heated to 60° C. for 1 h. The reaction was diluted with ethyl acetate and washed with water. The organic layers were combined, dried with sodium sulfate, filtered, concentrated, and purified with silica gel chromatography to afford the desired product (525 mg, 94%) as an light brown. ESI MS m/z 597 [M+1]+.

### Step 6. tert-butyl (17-(2-((6-(aminomethyl)-1H-indol-1-yl)methyl)-1H-imidazol-1-yl)-13-oxo-3,6,9-trioxa-12-azaheptadecyl)carbamate (JRW-2296)

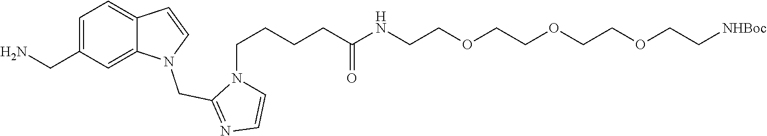 [SMILES: CC(C)(C)OC(=O)NCCOCCOCCOCCNC(=O)CCCCn1ccnc1Cn1ccc2ccc(CN)cc21]

To a solution of tert-butyl (17-(2-((6-cyano-1H-indol-1-yl)methyl)-1H-imidazol-1-yl)-13-oxo-3,6,9-trioxa-12-azaheptadecyl)carbamate (0.52 g, 0.88 mmol) in ammonia in methanol (7 N, 20 mL), a scoop of Rainey Nickel suspended in water was added. The reaction was charged with hydrogen (60 psi) and stirred at rt for 18 h. After degassing with nitrogen, the mixture was filtered over celite. The filtrate was concentrated to afford the crude product (0.49 g) as a semisolid. ESI MS m/z 601 [M+1]+.

### Step 7. tert-butyl (17-(2-((6-((((3-(6-hydroxy-3-oxoisoindolin-1-yl)-1H-indol-2-yl)methyl)amino)methyl)-1H-indol-1-yl)methyl)-1H-imidazol-1-yl)-13-oxo-3,6,9-trioxa-12-azaheptadecyl)carbamate (JRW-2300)

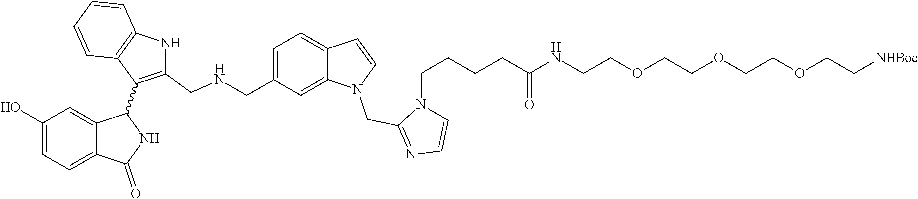 [SMILES: CC(C)(C)OC(=O)NCCOCCOCCOCCNC(=O)CCCCn1ccnc1Cn1ccc2ccc(CNCc3[nH]c4ccccc4c3C3NC(=O)c4ccc(O)cc43)cc21]

To a suspension of tert-butyl (17-(2-((6-(aminomethyl)-1H-indol-1-yl)methyl)-1H-imidazol-1-yl)-13-oxo-3,6,9-trioxa-12-azaheptadecyl)carbamate (0.45 g, 0.75 mmol) in THF (10 mL), 3-(6-hydroxy-3-oxoisoindolin-1-yl)-1H-indole-2-carbaldehyde (0.22 g, 0.75 mmol) was added. The reaction stirred at rt for 1 h. Sodium triacetoxyborohydride (0.48 g, 2.3 mmol) was added and stirred at rt for 3 h. The reaction was diluted with methanol, celite was added, concentrated, and purified with silica gel chromatography to afford the desired product (0.53 g, 80%) as a brown foam. ESI MS m/z 878 [M+1]+.

### Step 8. N-(2-(2-(2-(2-aminoethoxy)ethoxy)ethoxy)ethyl)-5-(2-((6-((((3-(6-hydroxy-3-oxoisoindolin-1-yl)-1H-indol-2-yl)methyl)amino)methyl)-1H-indol-1-yl)methyl)-1H-imidazol-1-yl)pentanamide (JRW-2309)

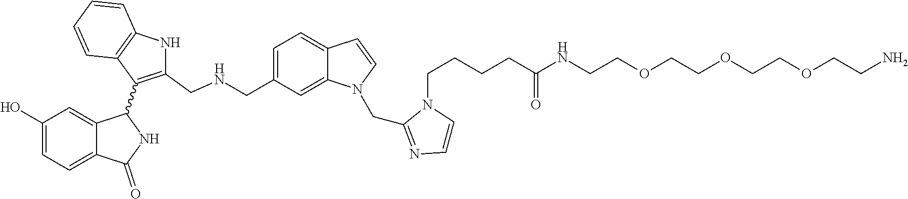 [SMILES: NCCOCCOCCOCCNC(=O)CCCCn1ccnc1Cn1ccc2ccc(CNCc3[nH]c4ccccc4c3C3NC(=O)c4ccc(O)cc43)cc21]

To a solution of tert-butyl (17-(2-((6-((((3-(6-hydroxy-3-oxoisoindolin-1-yl)-1H-indol-2-yl)methyl)amino)methyl)-1H-indol-1-yl)methyl)-1H-imidazol-1-yl)-13-oxo-3,6,9-trioxa-12-azaheptadecyl)carbamate (0.030 g, 0.034 mmol) in dichloromethane (10 mL), trifluoroacetic acid (1 mL) was added. The reaction stirred at rt for 1.5 h. The mixture was concentrated to give crude product as a colorless oil. ESI MS m/z 777 [M+1]+.

### Step 9. N-(15-(5,5-difluoro-7-(1H-pyrrol-2-yl)-5H-5λ⁴,6λ⁴-dipyrrolo[1,2-c:2′,1′-f][1,3,2]diazaborinin-3-yl)-13-oxo-3,6,9-trioxa-12-azapentadecyl)-5-(2-((6-((((3-(6-hydroxy-3-oxoisoindolin-1-yl)-1H-indol-2-yl)methyl)amino)methyl)-1H-indol-1-yl)methyl)-1H-imidazol-1-yl)pentanamide (JRW-2310)

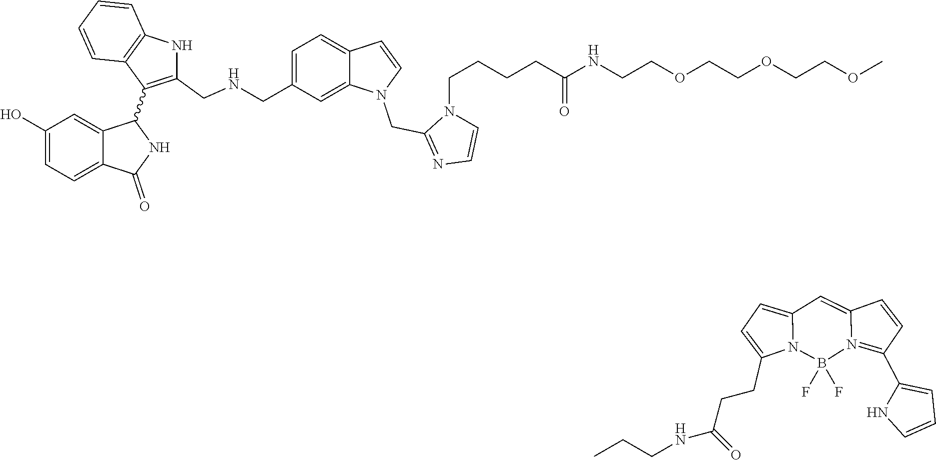

To a solution of N-(2-(2-(2-(2-aminoethoxy)ethoxy)ethoxy)ethyl)-5-(2-((6-((((3-(6-hydroxy-3-oxoisoindolin-1-yl)-1H-indol-2-yl)methyl)amino)methyl)-1H-indol-1-yl)methyl)-1H-imidazol-1-yl)pentanamide (26 mg, 0.033 mmol) in DMF (2 mL), 2,5-dioxopyrrolidin-1-yl 3-(5,5-difluoro-7-(1H-pyrrol-2-yl)-5H-5λ⁴,6λ⁴-dipyrrolo[1,2-c:2′,1′-f][1,3,2]diazaborinin-3-yl)propanoate (14 mg, 0.033 mmol) and diisopropylethylamine (34 mg, 0.27 mmol) was added. The reaction was stirred at rt for 30 min. The mixture was diluted with methanol and purified by reverse phase preparative HPLC to afford the desired product (38 mg, quant) as a purple solid. ESI MS m/z 1088 [M+1]+.

### N-(15-(5,5-difluoro-7-(1H-pyrrol-2-yl)-5H-5λ⁴,6λ⁴-dipyrrolo[1,2-c:2′,1′-f][1,3,2]diazaborinin-3-yl)-13-oxo-3,6,9-trioxa-12-azapentadecyl)-5-(4-((6-((((3-(6-hydroxy-3-oxoisoindolin-1-yl)-1H-indol-2-yl)methyl)amino)methyl)-1H-indol-1-yl)methyl)-1H-imidazol-1-yl)pentanamide (JRW-2308)

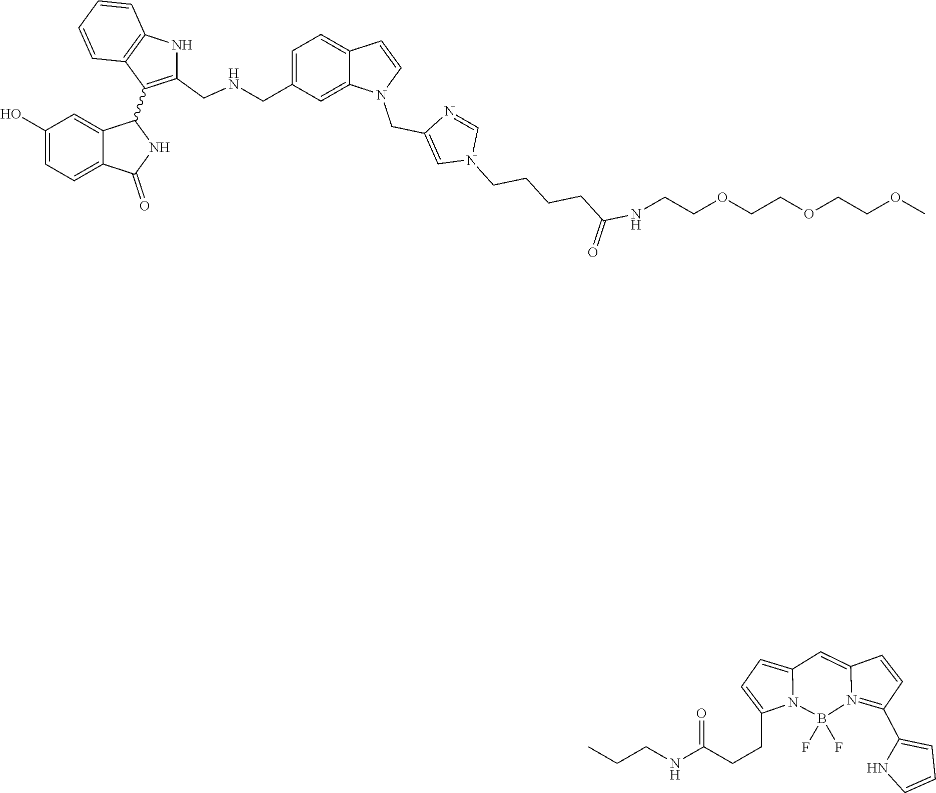

### Step 1. Methyl 5-(4-((6-cyano-1H-indol-1-yl)methyl)-1H-imidazol-1-yl)pentanoate (JRW-2241)

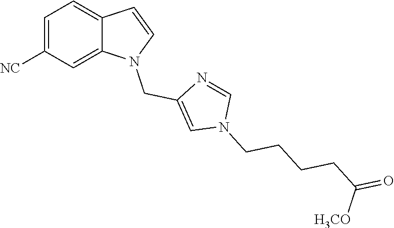 [SMILES: COC(=O)CCCCn1cnc(Cn2ccc3ccc(C#N)cc32)c1]

To a solution of 1H-indole-6-carbonitrile (150 mg, 1.1 mmol) in DMF (20 mL) chilled with an ice bath, sodium hydride (63 mg, 1.6 mmol, 60%), methyl 5-(4-(chloromethyl)-1H-imidazol-1-yl)pentanoate hydrochloride (282 mg, 1.1 mmol), and tetrabutylammonium iodide (39 mg, 0.11 mmol) was added. The mixture stirred at 0° C. for 30 min and rt for 2 h. The reaction was diluted with ethyl acetate and washed with water. The organic layers were combined, dried with sodium sulfate, filtered, concentrated, and purified with silica gel chromatography to afford the desired product (330 mg, 93%). ESI MS m/z 337 [M+1]+.

### Step 2. 5-(4-((6-cyano-1H-indol-1-yl)methyl)-1H-imidazol-1-yl)pentanoic acid (JRW-2243)

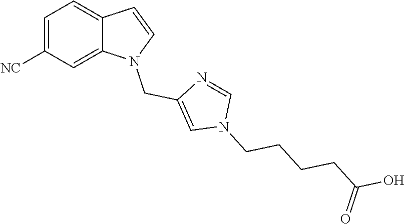 [SMILES: N#Cc1ccc2ccn(Cc3cn(CCCCC(=O)O)cn3)c2c1]

To a solution of methyl 5-(4-((6-cyano-1H-indol-1-yl)methyl)-1H-imidazol-1-yl)pentanoate (0.33 g, 0.98 mmol) in dioxane (10 mL), lithium hydroxide (0.12 g, 4.9 mmol) and water (1 mL) was added. The reaction was heated to 40° C. for 18 h. The mixture diluted with water, the pH was adjusted to 3 with HCl, and extracted with CHCl₃/IPA 3:1. Concentration of the organic layer afforded the crude product (0.36 g) as a colorless oil. ESI MS m/z 323 [M+1]+.

### Step 3. tert-butyl (17-(4-((6-cyano-1H-indol-1-yl)methyl)-1H-imidazol-1-yl)-13-oxo-3,6,9-trioxa-12-azaheptadecyl)carbamate (JRW-2245)

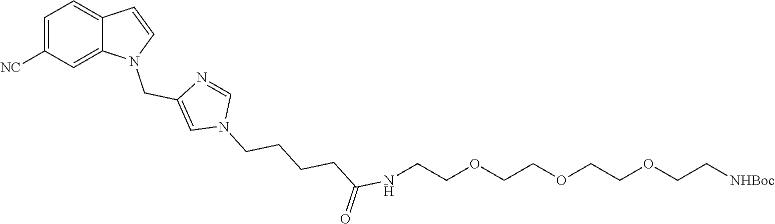 [SMILES: CC(C)(C)OC(=O)NCCOCCOCCOCCNC(=O)CCCCn1cnc(Cn2ccc3ccc(C#N)cc32)c1]

To a solution of 5-(4-((6-cyano-1H-indol-1-yl)methyl)-1H-imidazol-1-yl)pentanoic acid (0.30 g, 0.93 mmol) in DMF (10 mL), tert-butyl (2-(2-(2-(2-aminoethoxy)ethoxy)ethoxy)ethyl)carbamate (0.27 g, 0.93 mmol), hydroxybenzotriazole (0.28 g, 1.9 mmol), 1-Ethyl-3-(3′-dimethylaminopropyl)carbodiimide, HCl (0.36 g, 1.9 mmol), and diisopropylamine (0.36 g, 2.8 mmol) was added. The reaction was heated to 60° C. for 2 h. The reaction was diluted with ethyl acetate and washed with water. The organic layers were combined, dried with sodium sulfate, filtered, concentrated, and purified with silica gel chromatography to afford the desired product (0.30 g, 54%) as a light yellow oil. ESI MS m/z 597 [M+1]+.

### Step 4. tert-butyl (17-(4-((6-(aminomethyl)-1H-indol-1-yl)methyl)-1H-imidazol-1-yl)-13-oxo-3,6,9-trioxa-12-azaheptadecyl)carbamate (JRW-2248)

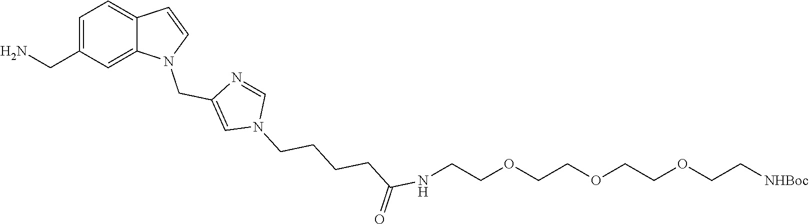 [SMILES: CC(C)(C)OC(=O)NCCOCCOCCOCCNC(=O)CCCCn1cnc(Cn2ccc3ccc(CN)cc32)c1]

To a solution of tert-butyl (17-(4-((6-cyano-1H-indol-1-yl)methyl)-1H-imidazol-1-yl)-13-oxo-3,6,9-trioxa-12-azaheptadecyl)carbamate (0.30 g, 0.50 mmol) in ammonia in methanol (7 N, 20 mL), a scoop of Rainey Nickel suspended in water was added. The reaction was charged with hydrogen (60 psi) and stirred at rt for 18 h. After degassing with nitrogen, the mixture was filtered over celite. The filtrate was concentrated to afford the crude product (0.31 g) as a colorless semisolid. ESI MS m/z 601 [M+1]+.

### Step 5. tert-butyl (17-(4-((6-((((3-(6-hydroxy-3-oxoisoindolin-1-yl)-1H-indol-2-yl)methyl)amino)methyl)-1H-indol-1-yl)methyl)-1H-imidazol-1-yl)-13-oxo-3,6,9-trioxa-12-azaheptadecyl)carbamate (JRW-2252)

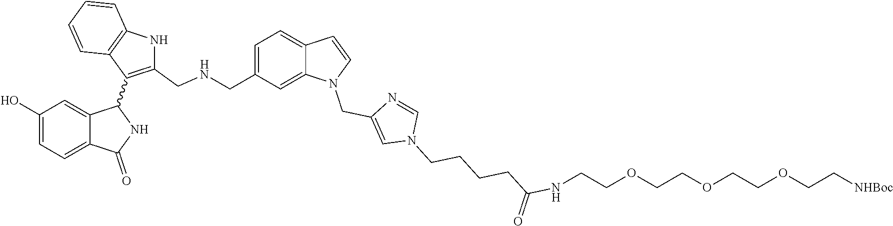 [SMILES: CC(C)(C)OC(=O)NCCOCCOCCOCCNC(=O)CCCCn1cnc(Cn2ccc3ccc(CNCc4[nH]c5ccccc5c4C4NC(=O)c5ccc(O)cc54)cc32)c1]

To a suspension of tert-butyl (17-(4-((6-(aminomethyl)-1H-indol-1-yl)methyl)-1H-imidazol-1-yl)-13-oxo-3,6,9-trioxa-12-azaheptadecyl)carbamate (0.31 g, 0.50 mmol) in THF (10 mL), 3-(6-hydroxy-3-oxoisoindolin-1-yl)-1H-indole-2-carbaldehyde (0.16 g, 0.55 mmol) was added. The reaction stirred at rt for 3 h. Sodium triacetoxyborohydride (0.58 g, 2.7 mmol) was added and stirred at rt for 18 h. The reaction was diluted with methanol, celite was added, concentrated, and purified with silica gel chromatography to afford the desired product (0.33 g, 68%) as a brown foam. ESI MS m/z 878 [M+1]+.

### Step 6. N-(2-(2-(2-(2-aminoethoxy)ethoxy)ethoxy)ethyl)-5-(4-((6-((((3-(6-hydroxy-3-oxoisoindolin-1-yl)-1H-indol-2-yl)methyl)amino)methyl)-1H-indol-1-yl)methyl)-1H-imidazol-1-yl)pentanamide (JRW-2305)

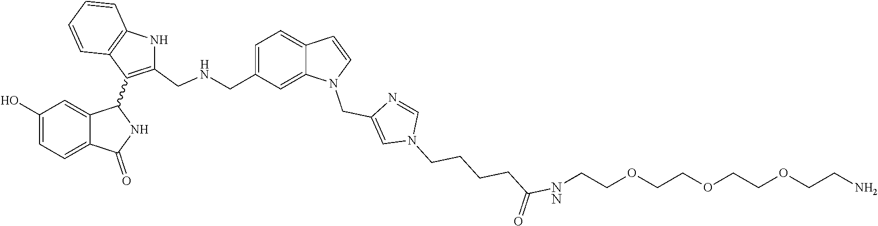

To a solution of tert-butyl (17-(4-((6-((((3-(6-hydroxy-3-oxoisoindolin-1-yl)-1H-indol-2-yl)methyl)amino)methyl)-1H-indol-1-yl)methyl)-1H-imidazol-1-yl)-13-oxo-3,6,9-trioxa-12-azaheptadecyl)carbamate (0.030 g, 0.034 mmol) in dichloromethane (10 mL), trifluoroacetic acid (1 mL) was added. The reaction stirred at rt for 1.5 h. The mixture was concentrated to give crude product as a colorless oil. ESI MS m/z 777 [M+1]+.

### Step 7. N-(15-(5,5-difluoro-7-(1H-pyrrol-2-yl)-5H-5λ⁴,6λ⁴-dipyrrolo[1,2-c:2′,1′-f][1,3,2]diazaborinin-3-yl)-13-oxo-3,6,9-trioxa-12-azapentadecyl)-5-(4-((6-((((3-(6-hydroxy-3-oxoisoindolin-1-yl)-1H-indol-2-yl)methyl)amino)methyl)-1H-indol-1-yl)methyl)-1H-imidazol-1-yl)pentanamide (JRW-2308)

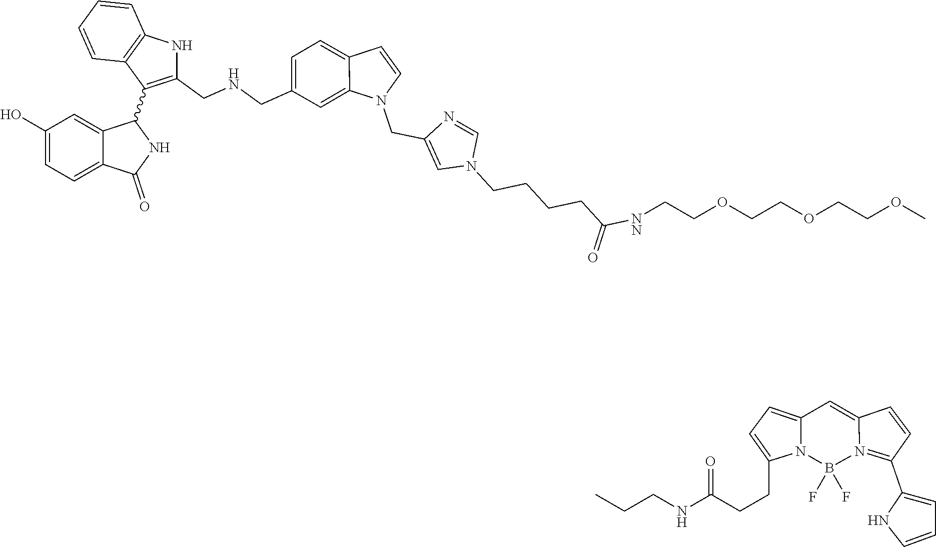

To a solution of N-(2-(2-(2-(2-aminoethoxy)ethoxy)ethoxy)ethyl)-5-(4-((6-((((3-(6-hydroxy-3-oxoisoindolin-1-yl)-1H-indol-2-yl)methyl)amino)methyl)-1H-indol-1-yl)methyl)-1H-imidazol-1-yl)pentanamide (26 mg, 0.033 mmol) in DMF (2 mL), 2,5-dioxopyrrolidin-1-yl 3-(5,5-difluoro-7-(1H-pyrrol-2-yl)-5H-5λ⁴,6λ⁴-dipyrrolo[1,2-c:2′,1′-f][1,3,2]diazaborinin-3-yl)propanoate (14 mg, 0.033 mmol) and diisopropylethylamine (34 mg, 0.27 mmol) was added. The reaction was stirred at rt for 30 min. The mixture was diluted with methanol and purified by reverse phase preparative HPLC to afford the desired product (40 mg, quant) as a purple solid. ESI MS m/z 1088 [M+1]+.

### 1-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-N-(3-(4-((6-((((3-(6-hydroxy-3-oxoisoindolin-1-yl)-1H-indol-2-yl)methyl)amino)methyl)-1H-indol-1-yl)methyl)-1H-1,2,3-triazol-1-yl)propyl)-3,6,9,12-tetraoxapentadecan-15-amide (JRW-2316)

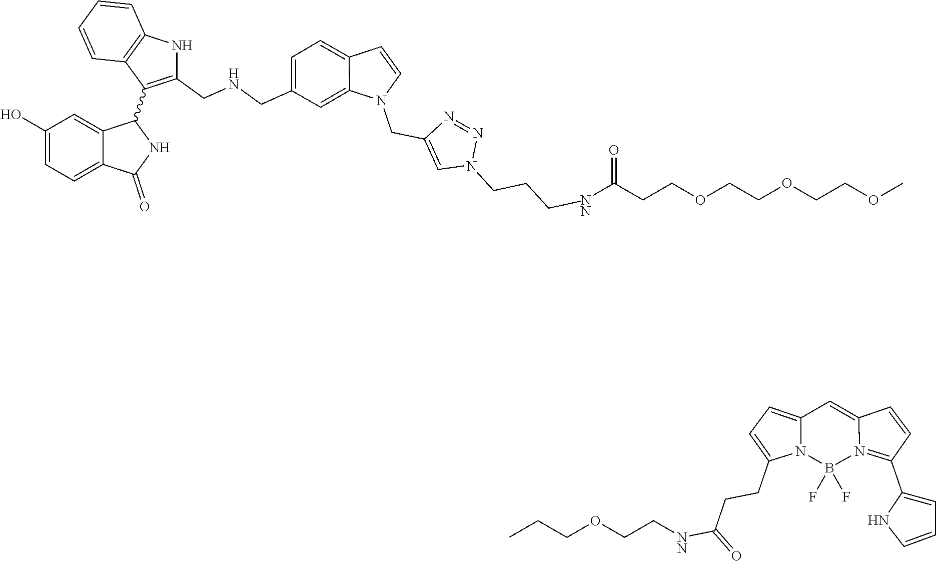

### Step 1. tert-butyl (3-(4-((6-cyano-1H-indol-1-yl)methyl)-1H-1,2,3-triazol-1-yl)propyl)carbamate (JRW-2293)

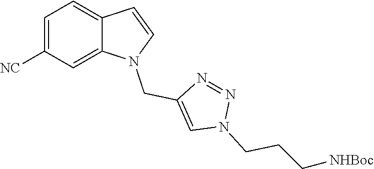 [SMILES: CC(C)(C)OC(=O)NCCCn1cc(Cn2ccc3ccc(C#N)cc32)nn1]

To a suspension of 1-(prop-2-yn-1-yl)-1H-indole-6-carbonitrile (0.18 g, 0.97 mmol) in tert-butanol (10 mL), tert-butyl (3-azidopropyl)carbamate (0.19 g, 0.97 mmol), copper sulfate (31 mg, 0.19 mmol), and sodium ascorbate (38 mg, 0.19 mmol) was added. Water (5 mL) was added, and the mixture stirred at rt for 18 h. The reaction was diluted with methanol, celite was added, concentrated, and purified with silica gel chromatography to afford the desired product (0.28 g, 74%) as a brown gum. ESI MS m/z 381 [M+1]+.

### Step 2. tert-butyl (3-(4-((6-(aminomethyl)-1H-indol-1-yl)methyl)-1H-1,2,3-triazol-1-yl)propyl)carbamate (JRW-2304)

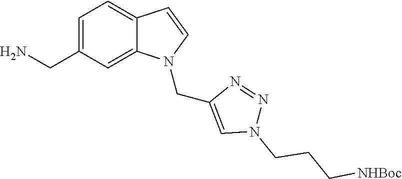 [SMILES: CC(C)(C)OC(=O)NCCCn1cc(Cn2ccc3ccc(CN)cc32)nn1]

To a solution of tert-butyl (3-(4-((6-cyano-1H-indol-1-yl)methyl)-1H-1,2,3-triazol-1-yl)propyl)carbamate (0.28 g, 0.72 mmol) in ammonia in methanol (7 N, 20 mL), a scoop of Rainey Nickel suspended in water was added. The reaction was charged with hydrogen (60 psi) and stirred at rt for 18 h. After degassing with nitrogen, the mixture was filtered over celite. The filtrate was concentrated to afford the crude product (0.30 g) as a colorless semisolid. ESI MS m/z 385 [M+1]+.

### Step 3. tert-butyl (3-(4-((6-((((3-(6-hydroxy-3-oxoisoindolin-1-yl)-1H-indol-2-yl)methyl)amino)methyl)-1H-indol-1-yl)methyl)-1H-1,2,3-triazol-1-yl)propyl)carbamate (JRW-2307)

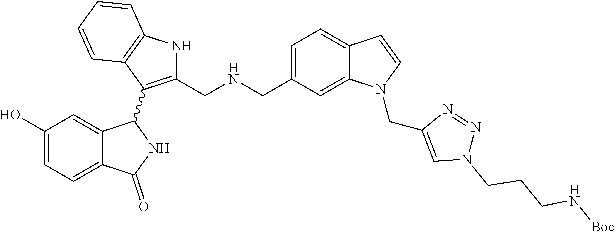 [SMILES: CC(C)(C)OC(=O)NCCCn1cc(Cn2ccc3ccc(CNCc4[nH]c5ccccc5c4C4NC(=O)c5ccc(O)cc54)cc32)nn1]

To a suspension of tert-butyl (3-(4-((6-(aminomethyl)-1H-indol-1-yl)methyl)-1H-1,2,3-triazol-1-yl)propyl)carbamate (0.27 g, 0.69 mmol) in THF (10 mL), 3-(6-hydroxy-3-oxoisoindolin-1-yl)-1H-indole-2-carbaldehyde (0.20 g, 0.68 mmol) was added. The reaction stirred at rt for 2 h. Sodium triacetoxyborohydride (0.58 g, 2.7 mmol) was added and stirred at rt for 2 h. The reaction was diluted with methanol, celite was added, concentrated, and purified with silica gel chromatography to afford the desired product (0.29 g, 64%) as a brown foam. ESI MS m/z 660 [M+1]+.

### Step 4. 3-(2-((((1-((1-(3-aminopropyl)-1H-1,2,3-triazol-4-yl)methyl)-1H-indol-6-yl)methyl)amino)methyl)-1H-indol-3-yl)-5-hydroxyisoindolin-1-one (JRW-2314)

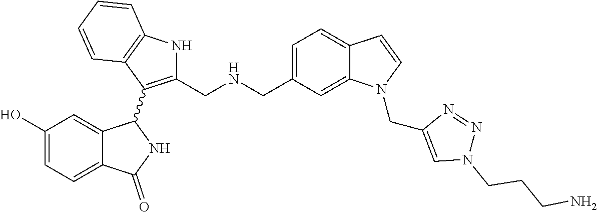 [SMILES: NCCCn1cc(Cn2ccc3ccc(CNCc4[nH]c5ccccc5c4C4NC(=O)c5ccc(O)cc54)cc32)nn1]

To a solution of tert-butyl (3-(4-((6-((((3-(6-hydroxy-3-oxoisoindolin-1-yl)-1H-indol-2-yl)methyl)amino)methyl)-1H-indol-1-yl)methyl)-1H-1,2,3-triazol-1-yl)propyl)carbamate (0.030 g, 0.045 mmol) in dichloromethane (10 mL), trifluoroacetic acid (1 mL) was added. The reaction stirred at rt for 1 h. The mixture was concentrated to give crude product as a colorless oil. ESI MS m/z 561 [M+1]+.

### Step 5. 1-(3-(5,5-difluoro-7-(1H-pyrrol-2-yl)-5H-5λ⁴,6λ⁴-dipyrrolo[1,2-c:2′,1′-f][1,3,2]diazaborinin-3-yl)propanamido)-N-(3-(4-((6-((((3-(6-hydroxy-3-oxoisoindolin-1-yl)-1H-indol-2-yl)methyl)amino)methyl)-1H-indol-1-yl)methyl)-1H-1,2,3-triazol-1-yl)propyl)-3,6,9,12-tetraoxapentadecan-15-amide (JRW-2316)

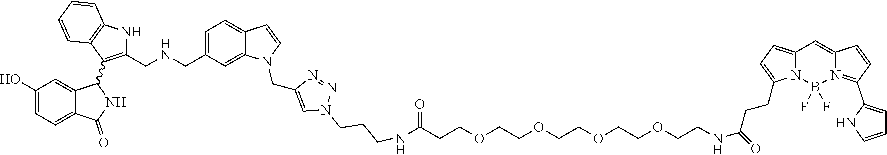

To a solution of 3-(2-((((1-((1-(3-aminopropyl)-1H-1,2,3-triazol-4-yl)methyl)-1H-indol-6-yl)methyl)amino)methyl)-1H-indol-3-yl)-5-hydroxyisoindolin-1-one (22 mg, 0.039 mmol) in DMF (2 mL), 2,5-dioxopyrrolidin-1-yl 3-(5,5-difluoro-7-(1H-pyrrol-2-yl)-5H-5λ⁴,6λ⁴-dipyrrolo[1,2-c:2′,1′-f][1,3,2]diazaborinin-3-yl)propanoate (13 mg, 0.019 mmol) and diisopropylethylamine (40 mg, 0.31 mmol) was added. The reaction was stirred at rt for 30 min. The mixture was diluted with methanol and purified by reverse phase preparative HPLC to afford the desired product (12 mg, 66%) as a purple solid. ESI MS m/z 1119 [M+1]+.

### N-(15-(5,5-difluoro-7-(1H-pyrrol-2-yl)-5H-5λ⁴,6λ⁴-dipyrrolo[1,2-c:2′,1′-f][1,3,2]diazaborinin-3-yl)-13-oxo-3,6,9-trioxa-12-azapentadecyl)-2-((6-((((3-(6-hydroxy-3-oxoisoindolin-1-yl)-1H-indol-2-yl)methyl)amino)methyl)-1H-indol-1-yl)methyl)isonicotinamide (JRW-2317)

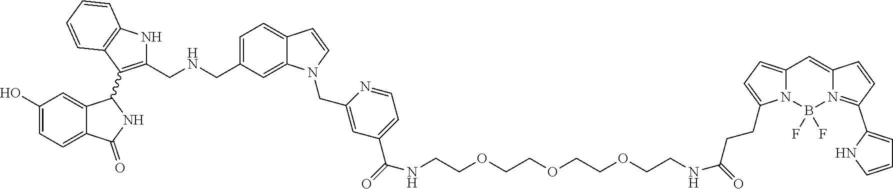

### Step 1. methyl 2-((6-cyano-1H-indol-1-yl)methyl)isonicotinate (JRW-2294)

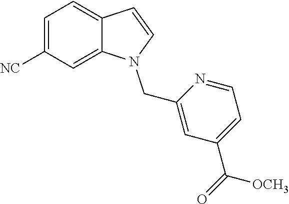 [SMILES: COC(=O)c1ccnc(Cn2ccc3ccc(C#N)cc32)c1]

To a solution of 1H-indole-6-carbonitrile (0.55 g, 3.9 mmol) in DMF (20 mL) chilled with an ice bath, sodium hydride (0.23 g, 5.8 mmol, 60%), methyl 2-(chloromethyl)isonicotinate hydrochloride (0.86 g, 3.9 mmol), and tetrabutylammonium iodide (142 mg, 0.39 mmol) was added. The mixture stirred at 0° C. for 1 h and rt for 3 h. The reaction was diluted with ethyl acetate and washed with water. The organic layers were combined, dried with sodium sulfate, filtered, concentrated, and purified with silica gel chromatography to afford the desired product (0.55 g, 48%) as a white solid. ESI MS m/z 292 [M+1]+.

### Step 2. 2-((6-Cyano-1H-indol-1-yl)methyl)isonicotinic acid (JRW-2295)

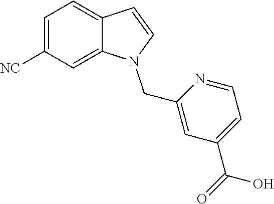 [SMILES: N#Cc1ccc2ccn(Cc3cc(C(=O)O)ccn3)c2c1]

To a solution of methyl 2-((6-cyano-1H-indol-1-yl)methyl)isonicotinate (0.55 g, 1.9 mmol) in dioxane (20 mL), lithium hydroxide (0.09 g, 3.8 mmol) and water (1 mL) was added. The reaction was heated to 40° C. for 4 h. The mixture diluted with water, the pH was adjusted to 3 with HCl, and extracted with CHCl₃/IPA 3:1. Concentration of the organic layer afforded the crude product (0.49 g) as a white solid. ESI MS m/z 278 [M+1]+.

### Step 3. Tert-butyl (1-(2-((6-cyano-1H-indol-1-yl)methyl)pyridin-4-yl)-1-oxo-5,8,11-trioxa-2-azatridecan-13-yl)carbamate (JRW-2298)

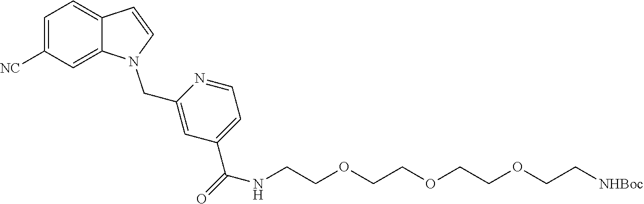 [SMILES: CC(C)(C)OC(=O)NCCOCCOCCOCCNC(=O)c1ccnc(Cn2ccc3ccc(C#N)cc32)c1]

To a solution of 2-((6-cyano-1H-indol-1-yl)methyl)isonicotinic acid (0.49 g, 1.8 mmol) in DMF (20 mL), tert-butyl (2-(2-(2-(2-aminoethoxy)ethoxy)ethoxy)ethyl)carbamate (0.62 g, 2.1 mmol), hydroxybenzotriazole (0.54 g, 3.5 mmol), 1-ethyl-3-(3′-dimethylaminopropyl)carbodiimide, HCl (0.68 g, 3.5 mmol), and diisopropylamine (0.68 g, 5.3 mmol) was added. The reaction was heated to 60° C. for 1 h. The reaction was diluted with ethyl acetate and washed with water. The organic layers were combined, dried with sodium sulfate, filtered, concentrated, and purified with silica gel chromatography to afford the desired product (0.89 g, 91%) as a white foam. ESI MS m/z 552 [M+1]+.

### Step 4. Tert-butyl (1-(2-((6-(aminomethyl)-1H-indol-1-yl)methyl)pyridin-4-yl)-1-oxo-5,8,11-trioxa-2-azatridecan-13-yl)carbamate (JRW-2306)

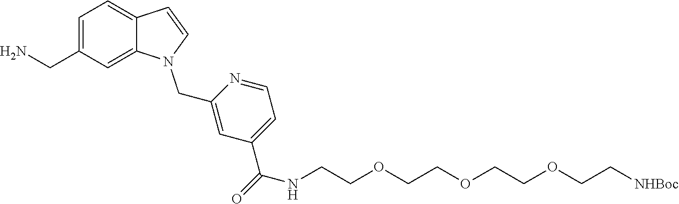 [SMILES: CC(C)(C)OC(=O)NCCOCCOCCOCCNC(=O)c1ccnc(Cn2ccc3ccc(CN)cc32)c1]

To a solution of tert-butyl (1-(2-((6-cyano-1H-indol-1-yl)methyl)pyridin-4-yl)-1-oxo-5,8,11-trioxa-2-azatridecan-13-yl)carbamate (0.89 g, 1.6 mmol) in ammonia in methanol (7 N, 20 mL), a scoop of Rainey Nickel suspended in water was added. The reaction was charged with hydrogen (60 psi) and stirred at rt for 18 h. After degassing with nitrogen, the mixture was filtered over celite. The filtrate was concentrated to afford the crude product (0.85 g) as a colorless oil. ESI MS m/z 556 [M+1]+.

### Step 5. Tert-butyl (1-(2-((6-((((3-(6-hydroxy-3-oxoisoindolin-1-yl)-1H-indol-2-yl)methyl)amino)methyl)-1H-indol-1-yl)methyl)pyridin-4-yl)-1-oxo-5,8,11-trioxa-2-azatridecan-13-yl)carbamate (JRW-2311)

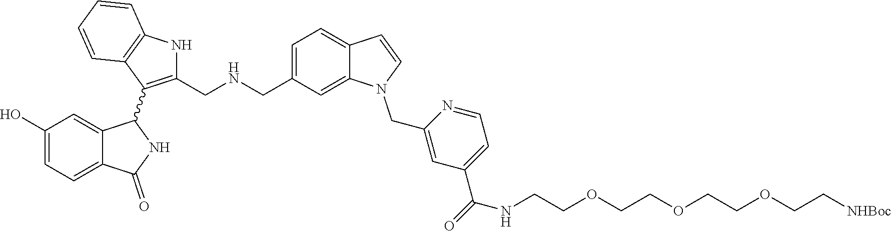 [SMILES: CC(C)(C)OC(=O)NCCOCCOCCOCCNC(=O)c1ccnc(Cn2ccc3ccc(CNCc4[nH]c5ccccc5c4C4NC(=O)c5ccc(O)cc54)cc32)c1]

To a suspension of tert-butyl (1-(2-((6-(aminomethyl)-1H-indol-1-yl)methyl)pyridin-4-yl)-1-oxo-5,8,11-trioxa-2-azatridecan-13-yl)carbamate (0.71 g, 1.3 mmol) in THF (10 mL), 3-(6-hydroxy-3-oxoisoindolin-1-yl)-1H-indole-2-carbaldehyde (0.25 g, 0.86 mmol) was added. The reaction stirred at rt for 1 h. Sodium triacetoxyborohydride (0.54 g, 2.6 mmol) was added and stirred at rt for 18 h. The reaction was diluted with methanol, celite was added, concentrated, and purified with silica gel chromatography to afford the desired product (0.38 g, 53%) as a brown foam. ESI MS m/z 832 [M+1]+.

### Step 6. N-(2-(2-(2-(2-aminoethoxy)ethoxy)ethoxy)ethyl)-2-((6-((((3-(6-hydroxy-3-oxoisoindolin-1-yl)-1H-indol-2-yl)methyl)amino)methyl)-1H-indol-1-yl)methyl)isonicotinamide (JRW-2315)

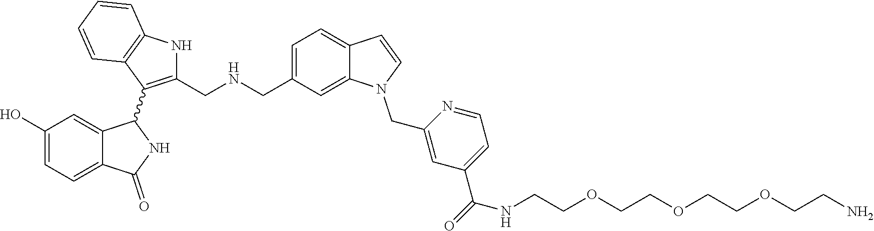 [SMILES: NCCOCCOCCOCCNC(=O)c1ccnc(Cn2ccc3ccc(CNCc4[nH]c5ccccc5c4C4NC(=O)c5ccc(O)cc54)cc32)c1]

To a solution of tert-butyl (1-(2-((6-((((3-(6-hydroxy-3-oxoisoindolin-1-yl)-1H-indol-2-yl)methyl)amino)methyl)-1H-indol-1-yl)methyl)pyridin-4-yl)-1-oxo-5,8,11-trioxa-2-azatridecan-13-yl)carbamate (0.030 g, 0.036 mmol) in dichloromethane (10 mL), trifluoroacetic acid (1 mL) was added. The reaction stirred at rt for 1 h. The mixture was concentrated to give crude product as an orange oil. ESI MS m/z 732 [M+1]+.

### Step 7. N-(15-(5,5-difluoro-7-(1H-pyrrol-2-yl)-5H-5λ⁴,6λ⁴-dipyrrolo[1,2-c:2′,1′-f][1,3,2]diazaborinin-3-yl)-13-oxo-3,6,9-trioxa-12-azapentadecyl)-2-((6-((((3-(6-hydroxy-3-oxoisoindolin-1-yl)-1H-indol-2-yl)methyl)amino)methyl)-1H-indol-1-yl)methyl)isonicotinamide (JRW-2317)

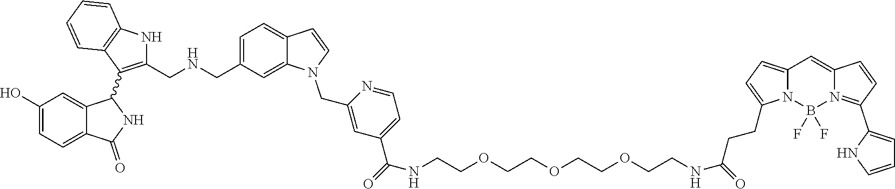

To a solution of N-(2-(2-(2-(2-aminoethoxy)ethoxy)ethoxy)ethyl)-2-((6-((((3-(6-hydroxy-3-oxoisoindolin-1-yl)-1H-indol-2-yl)methyl)amino)methyl)-1H-indol-1-yl)methyl)isonicotinamide (25 mg, 0.034 mmol) in DMF (2 mL), 2,5-dioxopyrrolidin-1-yl 3-(5,5-difluoro-7-(1H-pyrrol-2-yl)-5H-5λ⁴,6λ⁴-dipyrrolo[1,2-c:2′,1′-f][1,3,2]diazaborinin-3-yl)propanoate (14 mg, 0.034 mmol), and diisopropylethylamine (35 mg, 0.27 mmol) was added. The reaction was stirred at rt for 30 min. The mixture was diluted with methanol and purified by reverse phase preparative HPLC to afford the desired product (37 mg, quant) as a purple solid. ESI MS m/z 1043 [M+1]+.

### N-(15-(5,5-difluoro-7-(1H-pyrrol-2-yl)-5H-5λ⁴,6λ⁴-dipyrrolo[1,2-c:2′,1′-f][1,3,2]diazaborinin-3-yl)-13-oxo-3,6,9-trioxa-12-azapentadecyl)-5-((6-((((3-(6-hydroxy-3-oxoisoindolin-1-yl)-1H-indol-2-yl)methyl)amino)methyl)-1H-indol-1-yl)methyl)thiophene-2-carboxamide (JRW-2313)

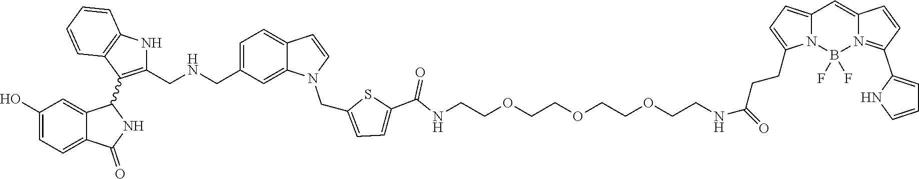

### Step 1. Methyl 5-((6-cyano-1H-indol-1-yl)methyl)thiophene-2-carboxylate (JRW-2283)

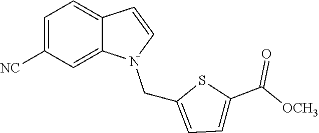 [SMILES: COC(=O)c1ccc(Cn2ccc3ccc(C#N)cc32)s1]

To a solution of 1H-indole-6-carbonitrile (0.70 g, 4.9 mmol) in DMF (20 mL) chilled with an ice bath, sodium hydride (0.29 g, 7.4 mmol, 60%), methyl 5-(chloromethyl)thiophene-2-carboxylate (0.94 g, 4.9 mmol), and tetrabutylammonium iodide (0.18 g, 0.49 mmol) was added. The mixture stirred at 0° C. for 1 h and rt for 1 h. The reaction was diluted with ethyl acetate and washed with water. The organic layers were combined, dried with sodium sulfate, filtered, concentrated, and purified with silica gel chromatography to afford the desired product (0.36 g, 26%). ESI MS m/z 297 [M+1]+.

### Step 2. 5-((6-cyano-1H-indol-1-yl)methyl)thiophene-2-carboxylic acid (JRW-2285)

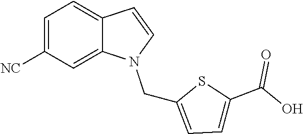 [SMILES: N#Cc1ccc2ccn(Cc3ccc(C(=O)O)s3)c2c1]

To a solution of methyl 5-((6-cyano-1H-indol-1-yl)methyl)thiophene-2-carboxylate (0.36 g, 1.2 mmol) in dioxane (20 mL), lithium hydroxide (0.14 g, 6.1 mmol) and water (1 mL) was added. The reaction was heated to 40° C. for 1.5 h. The mixture diluted with water, the pH was adjusted to 3 with HCl, and extracted with CHCl3/IPA 3:1. Concentration of the organic layer afforded the crude product (0.37 g) as a light solid. ESI MS m/z 283 [M+1]+.

### Step 3. Tert-butyl (1-(5-((6-cyano-1H-indol-1-yl)methyl)thiophen-2-yl)-1-oxo-5,8,11-trioxa-2-azatridecan-13-yl)carbamate (JRW-2287)

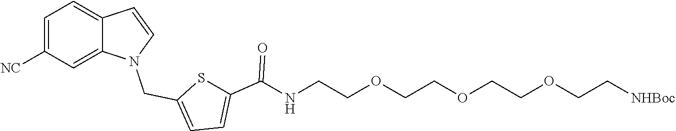 [SMILES: CC(C)(C)OC(=O)NCCOCCOCCOCCNC(=O)c1ccc(Cn2ccc3ccc(C#N)cc32)s1]

To a solution of 5-((6-cyano-1H-indol-1-yl)methyl)thiophene-2-carboxylic acid (0.34 g, 1.2 mmol) in DMF (20 mL), tert-butyl (2-(2-(2-(2-aminoethoxy)ethoxy)ethoxy)ethyl)carbamate (0.42 g, 1.4 mmol), hydroxybenzotriazole (0.37 g, 2.4 mmol), 1-ethyl-3-(3′-dimethylaminopropyl)carbodiimide, HCl (0.46 g, 2.4 mmol), and diisopropylamine (0.47 g, 3.6 mmol) was added. The reaction was heated to 60° C. for 1 h. The reaction was diluted with ethyl acetate and washed with water. The organic layers were combined, dried with sodium sulfate, filtered, concentrated, and purified with silica gel chromatography to afford the desired product (0.46 g, 73%) as a light yellow gum. ESI MS m/z 557 [M+1]+.

### Step 4. Tert-butyl (1-(5-((6-(aminomethyl)-1H-indol-1-yl)methyl)thiophen-2-yl)-1-oxo-5,8,11-trioxa-2-azatridecan-13-yl)carbamate (JRW-2301)

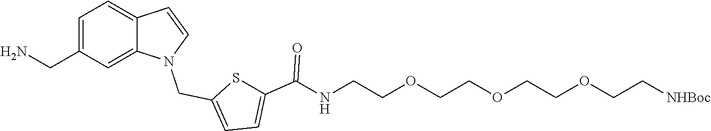 [SMILES: CC(C)(C)OC(=O)NCCOCCOCCOCCNC(=O)c1ccc(Cn2ccc3ccc(CN)cc32)s1]

To a solution of tert-butyl (1-(5-((6-cyano-1H-indol-1-yl)methyl)thiophen-2-yl)-1-oxo-5,8,11-trioxa-2-azatridecan-13-yl)carbamate (0.46 g, 0.82 mmol) in ammonia in methanol (7 N, 20 mL), a scoop of Rainey Nickel suspended in water was added. The reaction was charged with hydrogen (60 psi) and stirred at rt for 18 h. After degassing with nitrogen, the mixture was filtered over celite. The filtrate was concentrated to afford the crude product (0.53 g) as a light green foam. ESI MS m/z 561 [M+1]+.

### Step 5. Tert-butyl (1-(5-((6-((((3-(6-hydroxy-3-oxoisoindolin-1-yl)-1H-indol-2-yl)methyl)amino)methyl)-1H-indol-1-yl)methyl)thiophen-2-yl)-1-oxo-5,8,11-trioxa-2-azatridecan-13-yl)carbamate (JRW-2303)

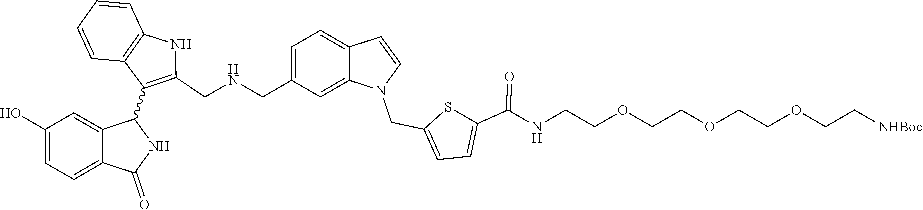 [SMILES: CC(C)(C)OC(=O)NCCOCCOCCOCCNC(=O)c1ccc(Cn2ccc3ccc(CNCc4[nH]c5ccccc5c4C4NC(=O)c5ccc(O)cc54)cc32)s1]

To a suspension of tert-butyl (1-(5-((6-(aminomethyl)-1H-indol-1-yl)methyl)thiophen-2-yl)-1-oxo-5,8,11-trioxa-2-azatridecan-13-yl)carbamate (0.42 g, 0.75 mmol) in THF (10 mL), 3-(6-hydroxy-3-oxoisoindolin-1-yl)-1H-indole-2-carbaldehyde (0.22 g, 0.75 mmol) was added. The reaction stirred at rt for 2 h. Sodium triacetoxyborohydride (0.48 g, 2.2 mmol) was added and stirred at rt for 3 d. The reaction was diluted with methanol, celite was added, concentrated, and purified with silica gel chromatography to afford the desired product (0.28 g, 44%) as a brown foam. ESI MS m/z 838 [M+1]+.

### Step 6. N-(2-(2-(2-(2-aminoethoxy)ethoxy)ethoxy)ethyl)-5-((6-((((3-(6-hydroxy-3-oxoisoindolin-1-yl)-1H-indol-2-yl)methyl)amino)methyl)-1H-indol-1-yl)methyl)thiophene-2-carboxamide (JRW-2312)

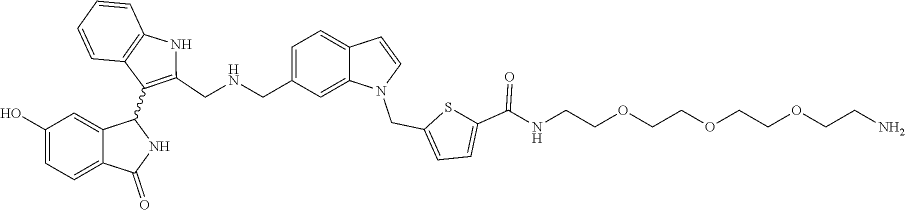 [SMILES: NCCOCCOCCOCCNC(=O)c1ccc(Cn2ccc3ccc(CNCc4[nH]c5ccccc5c4C4NC(=O)c5ccc(O)cc54)cc32)s1]

To a solution of tert-butyl (1-(5-((6-((((3-(6-hydroxy-3-oxoisoindolin-1-yl)-1H-indol-2-yl)methyl)amino)methyl)-1H-indol-1-yl)methyl)thiophen-2-yl)-1-oxo-5,8,11-trioxa-2-azatridecan-13-yl)carbamate (0.030 g, 0.036 mmol) in dichloromethane (10 mL), trifluoroacetic acid (1 mL) was added. The reaction stirred at rt for 1 h. The mixture was concentrated to give crude product as an orange oil. ESI MS m/z 737 [M+1]+.

### Step 7. N-(15-(5,5-difluoro-7-(1H-pyrrol-2-yl)-5H-5λ⁴,6λ⁴-dipyrrolo[1,2-c:2′,1′-f][1,3,2]diazaborinin-3-yl)-13-oxo-3,6,9-trioxa-12-azapentadecyl)-5-((6-((((3-(6-hydroxy-3-oxoisoindolin-1-yl)-1H-indol-2-yl)methyl)amino)methyl)-1H-indol-1-yl)methyl)thiophene-2-carboxamide (JRW-2313)

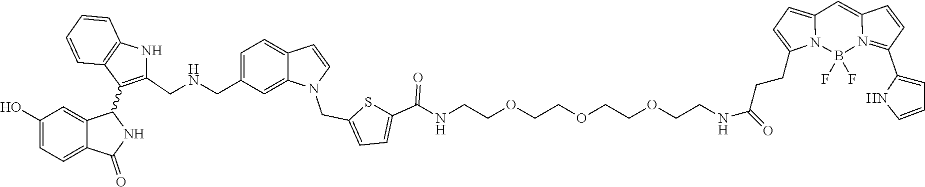

To a solution of N-(2-(2-(2-(2-aminoethoxy)ethoxy)ethoxy)ethyl)-5-((6-((((3-(6-hydroxy-3-oxoisoindolin-1-yl)-1H-indol-2-yl)methyl)amino)methyl)-1H-indol-1-yl)methyl)thiophene-2-carboxamide (25 mg, 0.034 mmol) in DMF (2 mL), 2,5-dioxopyrrolidin-1-yl 3-(5,5-difluoro-7-(1H-pyrrol-2-yl)-5H-5λ⁴,6λ⁴-dipyrrolo[1,2-c:2′,1′-f][1,3,2]diazaborinin-3-yl)propanoate (14 mg, 0.034 mmol) and diisopropylethylamine (35 mg, 0.27 mmol) was added. The reaction was stirred at rt for 30 min. The mixture was diluted with methanol and purified by reverse phase preparative HPLC to afford the desired product (25 mg, 71%) as a purple solid. ESI MS m/z 1048 [M+1]+.

### (6-(2-((22-(2-((6-((((3-(6-hydroxy-3-oxoisoindolin-1-yl)-1H-indol-2-yl)methyl)amino)methyl)-1H-indol-1-yl)methyl)-1H-imidazol-1-yl)-4,18-dioxo-8,11,14-trioxa-5,17-diazadocosyl)(methyl)carbamoyl)phenyl)-2,2,10,10-tetramethyl-1,11-bis(3-sulfamoylpropyl)-1,2,10,11-tetrahydro-13λ³-pyrano[3,2-g:5,6-g′]diquinoline-4,8-diyl)dimethanesulfonic acid (JRW-2395)

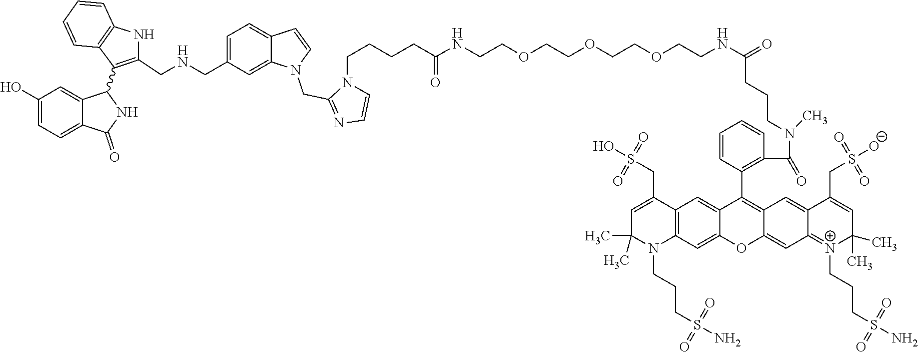 [SMILES: CN(CCCC(=O)NCCOCCOCCOCCNC(=O)CCCCn1ccnc1Cn1ccc2ccc(CNCc3[nH]c4ccccc4c3C3NC(=O)c4ccc(O)cc43)cc21)C(=O)c1ccccc1C1=c2cc3c(cc2Oc2cc4c(cc21)C(CS(=O)(=O)O)=CC(C)(C)N4CCCS(N)(=O)=O)=[N+](CCCS(N)(=O)=O)C(C)(C)C=C3CS(=O)(=O)[O-]]

To a solution of N-(2-(2-(2-(2-aminoethoxy)ethoxy)ethoxy)ethyl)-5-(2-((6-((((3-(6-hydroxy-3-oxoisoindolin-1-yl)-1H-indol-2-yl)methyl)amino)methyl)-1H-indol-1-yl)methyl)-1H-imidazol-1-yl)pentanamide (15 mg, 0.019 mmol) in DMF (2 mL), sodium (6-(2-((4-((2,5-dioxopyrrolidin-1-yl)oxy)-4-oxobutyl)(methyl)carbamoyl)phenyl)-2,2,10,10-tetramethyl-1,11-bis(3-sulfamoylpropyl)-8-(sulfomethyl)-1,2,10,11-tetrahydro-13λ³-pyrano[3,2-g:5,6-g′]diquinolin-4-yl)methanesulfonate (10 mg, 0.009 mmol) and diisopropylethylamine (20 mg, 0.15 mmol) was added. The reaction was stirred at rt for 30 min. The mixture was diluted with methanol and purified by reverse phase preparative HPLC to afford the desired product (7 mg, 43%) as a purple solid. ESI MS m/z 1751 [M+1]+.

### Example 2

### Measuring KRAS Target Engagement in Cells

Luminescence was produced from either NanoLuc (Nluc) tagging of KRAS as the BRET donor, or by NanoBiT tagging of KRAS, wherein BRET donor signal originates from KRAS multimeric species in cells. In 96-well plates, 20,000 HEK293 cells per well were transfected with KRAS-Nluc fusions or KRAS-NanoBiT fusions expressed from pFN31K and pFN32K plasmids. Transfections were performed using 3:1 FuGENE HD:plasmid ratios. 24 hours post transfection, cells were treated with Compound JRW-2111 or JRW-2025 and varying concentrations of test compounds. Test compounds included BI-2852 (a switch I/II site inhibitor), AMG-510 (a switch II site inhibitor that covalently modifies the Cys-12 residue of KRAS^{G12C}), and ARS-1620 (a switch II site inhibitor that covalently modifies the Cys-12 residue of KRAS^{G12C}). After incubation, NanoBRET-TE substrate solution was added to a final concentration of 1×, and BRET was measured on a Glomax Discover plate reader.

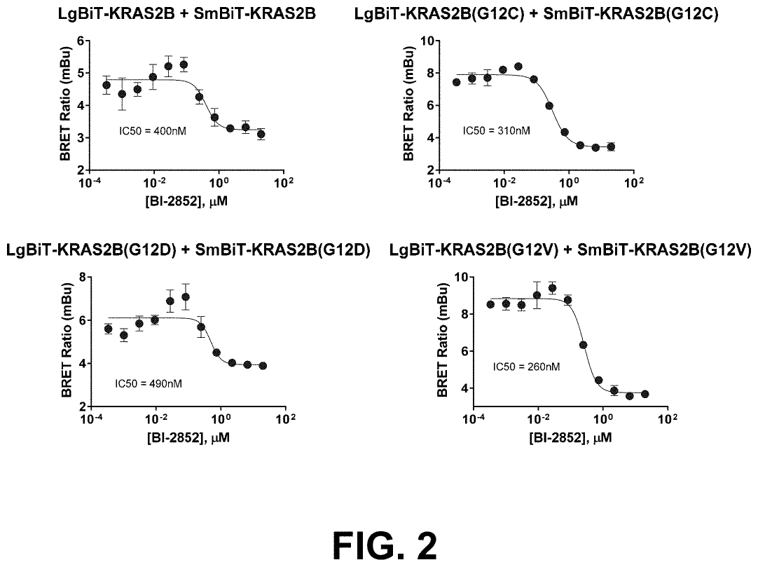

Data are shown in FIGS. 2-4. In particular, FIG. 2 shows data from a NanoBiT assay in cells expressing KRAS or a mutant thereof (KRAS^{G12C}, KRAS^{G12D}, or KRAS^{G12V}) as fusions with LgBiT and SmBiT. Using the compound JRW-2111 as the KRAS binding agent and the compound BI-2852 as the candidate KRAS binding compound, target engagement was observed for wild-type KRAS and all three mutants.

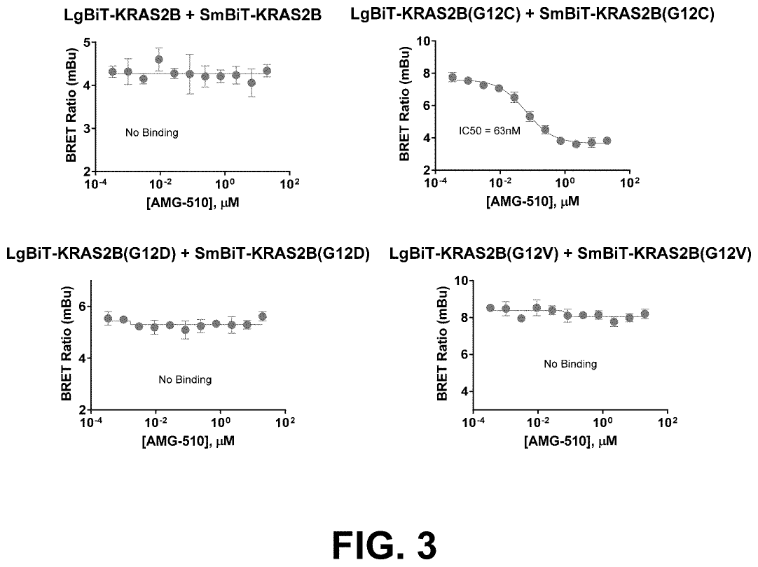

FIG. 3 shows data from a NanoBiT assay in cells expressing KRAS or a mutant thereof (KRAS^{G12C}, KRAS^{G12D}, or KRAS^{G12V}) as fusions with LgBiT and SmBiT. Using the compound JRW-2111 as the KRAS binding agent and the compound AMG-510, target engagement was only observed for the KRAS^{G12C} variant.

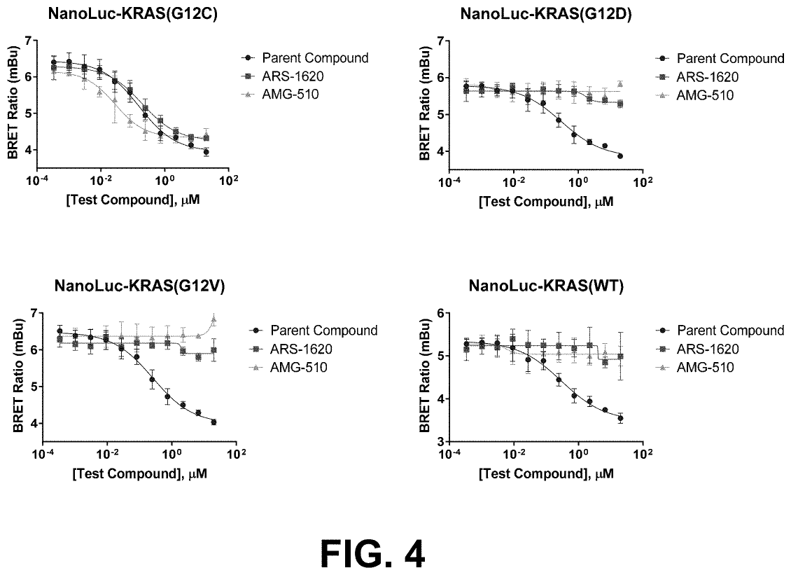

FIG. 4 shows data from cells expressing KRAS or a mutant thereof (e.g., KRAS^{G12C}, KRAS^{G12D}, or KRAS^{G12V}) as a fusion with NanoLuc. Using the compound JRW-2025 as the KRAS binding agent, competition was observed for BI-2852 for wild-type KRAS and the three KRAS variants, but competition was only observed for the KRAS^{G12C} mutant with compounds AMG-510 and ARS-1620.

In summary, at the oncogenic variant KRAS^{G12C}, target engagement could be observed for two inhibitor mechanisms, both with BI-2852 (a switch I/II site inhibitor) as well as AMG-510 and ARS1620 (switch II site inhibitors that react covalently with the cysteine at residue 12). This indicates that engagement of the switch I/II domain and switch II domain are mutually exclusive. These results support a broadly useful target engagement assay to query multiple engagement mechanisms at KRAS in live cells.

### Example 3

### Measuring Target Engagement at KRAS(G12V) Homomultimeric Complexes in Cells Using Enzyme Complementation-NanoBiT Oligomer Configuration with SmBiT Tag

It is postulated that KRAS may pre-exist in cells as a multimeric complex, which suggests that target engagement could be queried at an oligomeric form of KRAS, using a BRET donor formed via enzyme complementation. To explore this concept, the NanoBiT™ Technology was used to observe KRAS multimeric (e.g., dimer) complexes by NanoBiT tagging of KRAS, wherein BRET donor signal originates from an oligomeric KRAS. In a tissue culture flask, HEK293 cells per well were transfected with KRAS-NanoBiT fusions, LgBiT-KRAS2B(G12V) and SmBiT-KRAS2B(G12V) expressed from pNB3K or pNB4K plasmids along with pGEM-3Z carrier DNA (1:1:8 ratio by mass). Transfections were performed using 3:1 FuGENE HD:plasmid ratios. 24 hours post transfection, cells were treated with compound JRW-2192 and BI-2852.

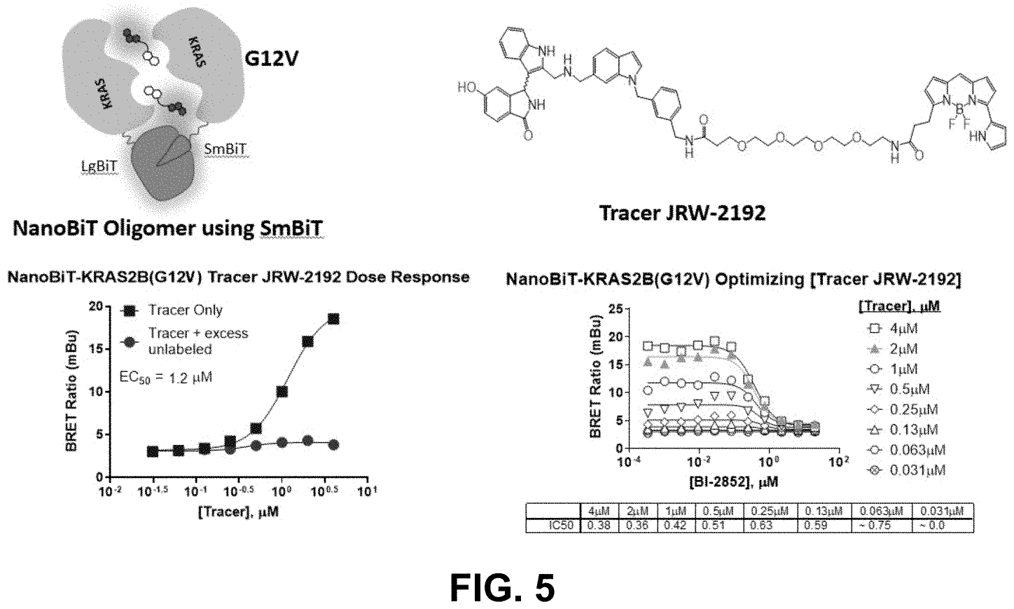

After incubation in live cells, NanoBRET-TE substrate solution was added to a final concentration of 1×, and BRET was measured on a Glomax Discover plate reader. Data are shown in FIG. 5. In particular, FIG. 5 shows data from the competition assay. Using the tracer JRW-2192 as the KRAS binding agent, increasing concentrations of the tracer demonstrate a dose-dependent increase in the BRET signal. BI-2852 shows a dose-dependent inhibition of the BRET signal induced by the tracer through functional competition.

### Example 4

### Measuring Target Engagement at KRAS(G12C) Homomultimeric Complexes in Cells Using Enzyme Complementation-NanoBiT Oligomer Configuration with SmBiT Tag

It is postulated that KRAS may pre-exist in cells as a multimeric complex, which suggests that target engagement could be queried at an oligomeric form of KRAS, using a BRET donor formed via enzyme complementation. To explore this concept, the NanoBiT™ Technology was used to observe KRAS multimeric (e.g., dimer) complexes by NanoBiT tagging of KRAS, wherein BRET donor signal originates from an oligomeric KRAS. In a tissue culture flask, HEK293 cells per well were transfected with KRAS-NanoBiT fusions, LgBiT-KRAS2B(G12C) and SmBiT-KRAS2B(G12C) expressed from pNB3K or pNB4K plasmids along with pGEM-3Z carrier DNA (1:1:8 ratio by mass). Transfections were performed using 3:1 FuGENE HD:plasmid ratios. 24 hours post transfection, cells were treated with compound JRW-2220 and BI-2852.

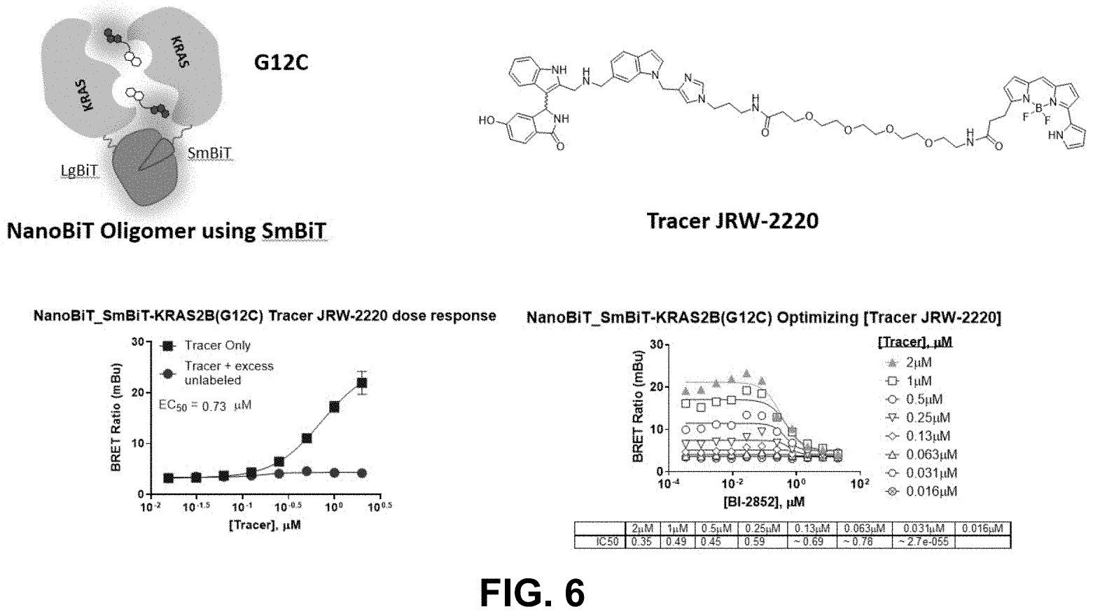

After incubation in live cells, NanoBRET-TE substrate solution was added to a final concentration of 1×, and BRET was measured on a Glomax Discover plate reader. Data are shown in FIG. 6. In particular, FIG. 6 shows data from the competition assay. Using the tracer JRW-2220 as the KRAS binding agent, increasing concentrations of the tracer demonstrate a dose-dependent increase in the BRET signal. BI-2852 shows a dose-dependent inhibition of the BRET signal induced by the tracer through functional competition.

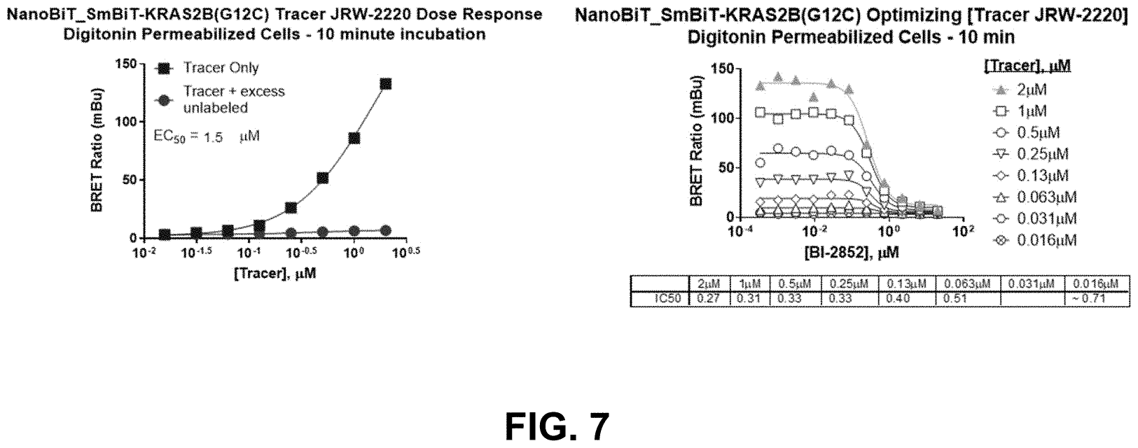

The assay was also performed in digitonin permeabilized cells. Permeabilization resulted in a drop in overall luminescence, but still allowed measurement of target engagement. Data are shown in FIG. 7. In particular, FIG. 7 shows data from the competition assay. Using the tracer JRW-2220 as the KRAS binding agent, increasing concentrations of the tracer demonstrate a dose-dependent increase in the BRET signal. BI-2852 shows a dose-dependent inhibition of the BRET signal induced by the tracer through functional competition in permeabilized cells.

### Example 5

### Measuring KRAS(G12C) Homomultimeric Complexes in Cells Using Enzyme Complementation-NanoBiT Oligomer Configuration with HiBiT Tag

It is postulated that KRAS may pre-exist in cells as a multimeric complex, which suggests that target engagement could be queried at an oligomeric form of KRAS, using a BRET donor formed via enzyme complementation. To explore this concept, the NanoBiT™ Technology was used to form KRAS multimeric (e.g., dimer) complexes by NanoBiT tagging of KRAS, wherein BRET donor signal originates from an oligomeric KRAS. In a tissue culture flask, HEK293 cells per well were transfected with KRAS-NanoBiT fusions, LgBiT-KRAS2B(G12C) and HiBiT-KRAS2B(G12C) expressed from pNB3K or pFN38A plasmids along with pGEM-3Z carrier DNA (1:1:8 ratio by mass). Transfections were performed using 3:1 FuGENE HD:plasmid ratios. 24 hours post transfection, cells were treated with compound JRW-2220 and BI-2852.

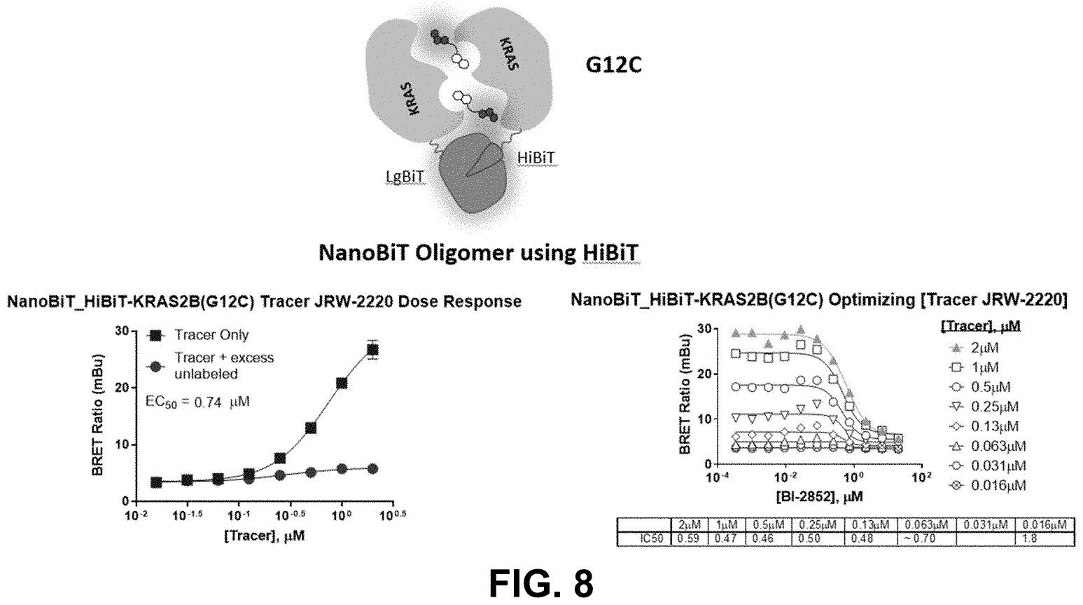

After incubation in live cells, NanoBRET-TE substrate solution was added to a final concentration of 1×, and BRET was measured on a Glomax Discover plate reader. Data are shown in FIG. 8. In particular, FIG. 8 shows data from the competition assay. Using the tracer JRW-2220 as the KRAS binding agent, increasing concentrations of the tracer demonstrate a dose-dependent increase in the BRET signal. BI-2852 shows a dose-dependent inhibition of the BRET signal induced by the tracer through functional competition.

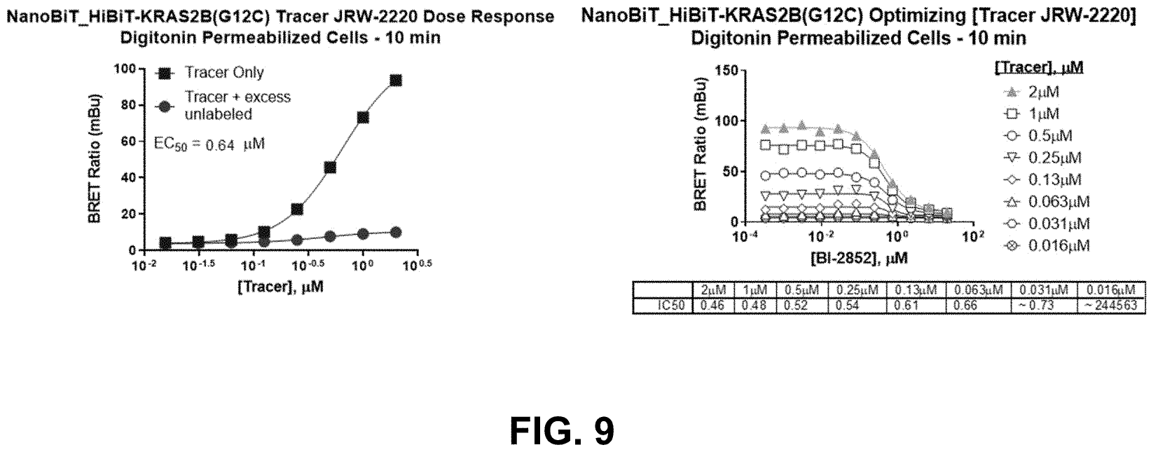

The assay was also performed in digitonin permeabilized cells. Even after permeabilization, donor signal levels remained significant due to the use of the HiBiT tag, facilitating measurement of target engagement. Data are shown in FIG. 9. In particular, FIG. 9 shows data from the competition assay. Using the tracer JRW-2220 as the KRAS binding agent, increasing concentrations of the tracer demonstrate a dose-dependent increase in the BRET signal. BI-2852 shows a dose-dependent inhibition of the BRET signal induced by the tracer through functional competition in permeabilized cells.

### Example 6

### Measuring KRAS2B, HRAS, or NRAS Target Engagement in Cells

It is postulated that many RAS variants may pre-exist in cells as a multimeric complex, which suggests that target engagement could be queried at an oligomeric form of RAS, using a BRET donor formed via enzyme complementation. To explore this concept, the NanoBiT™ Technology was used to form RAS multimeric (e.g., dimer) complexes by NanoBiT tagging of RAS, wherein BRET donor signal originates from an oligomeric RAS. In a tissue culture flask, HEK293 cells per well were transfected with RAS-NanoBiT fusions, LgBiT-RAS and SmBiT-RAS expressed from pNB3K or pNB4K plasmids along with pGEM-3Z carrier DNA (1:1:8 ratio by mass). Transfections were performed using 3:1 FuGENE HD:plasmid ratios. 24 hours post transfection, cells were treated with compound JRW-2219, JRW-2220, or JRW-2310, and BI-2852.

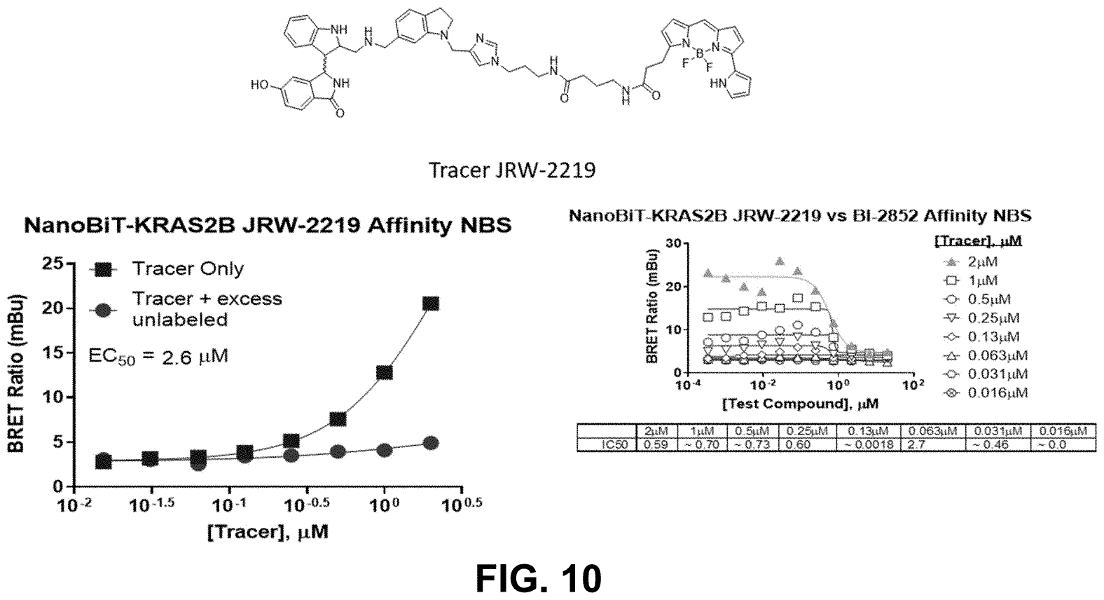

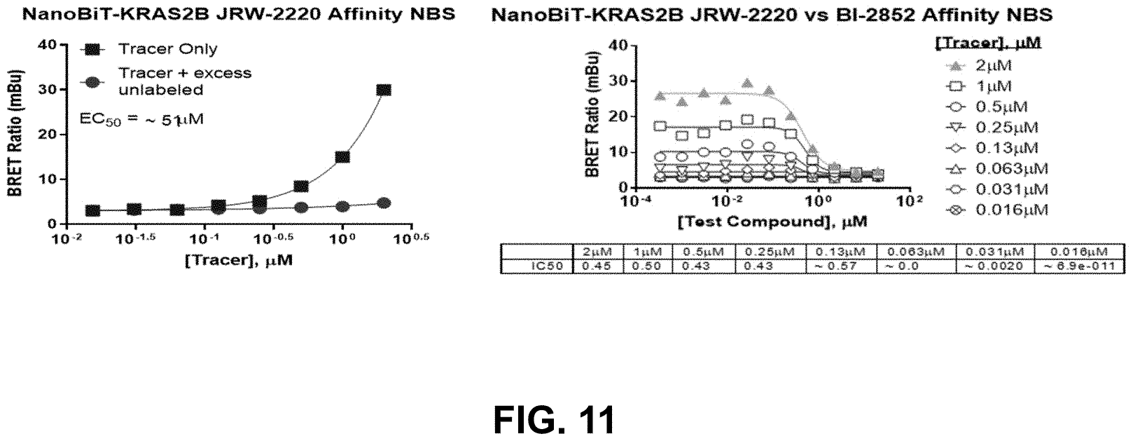

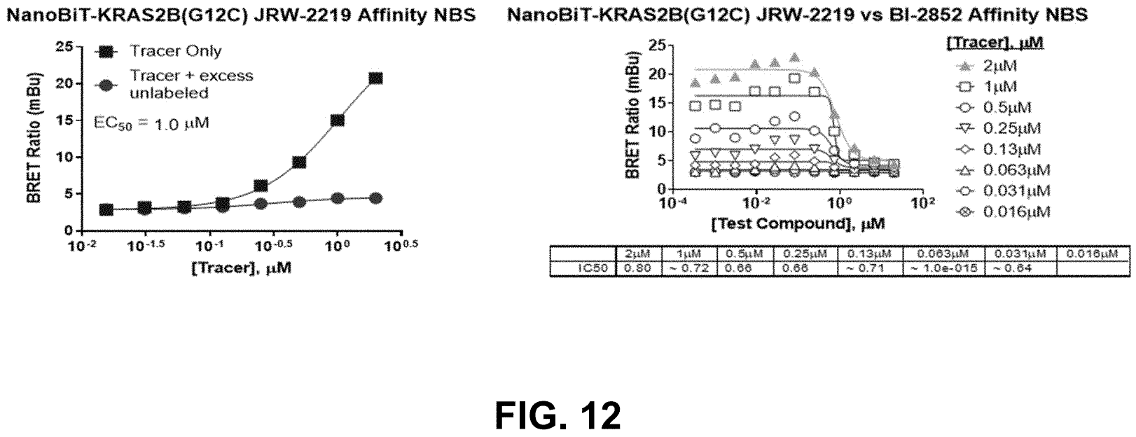

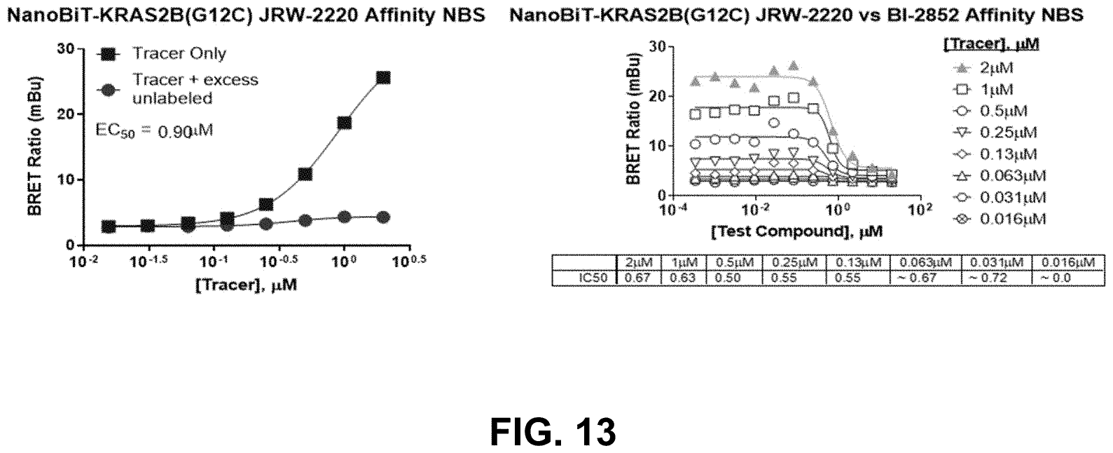

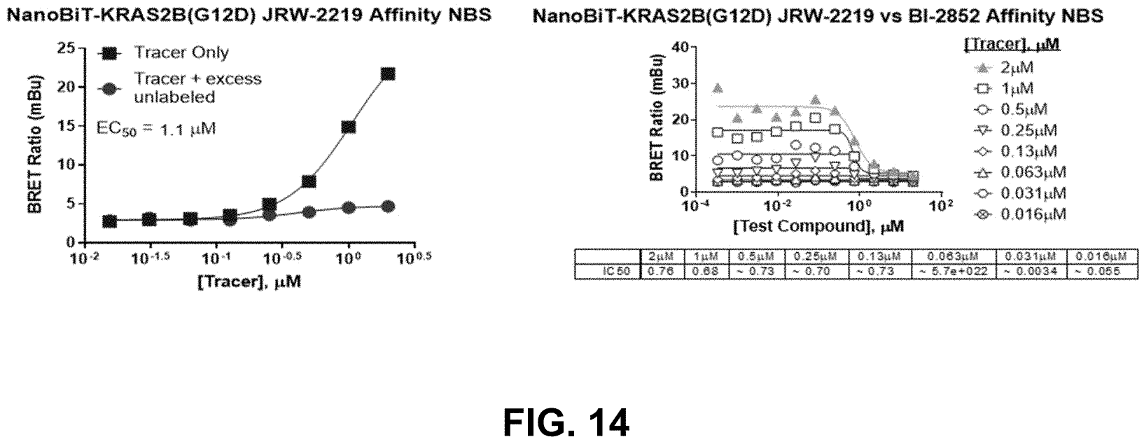

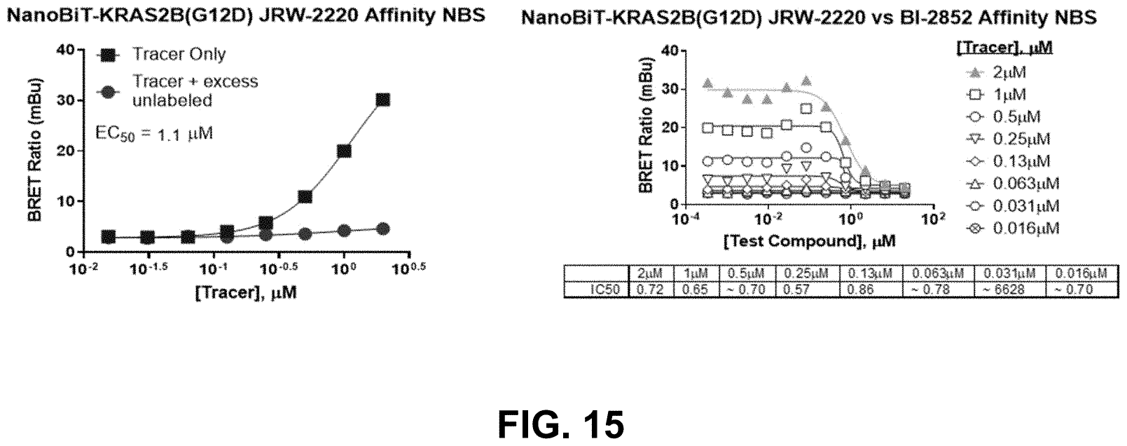

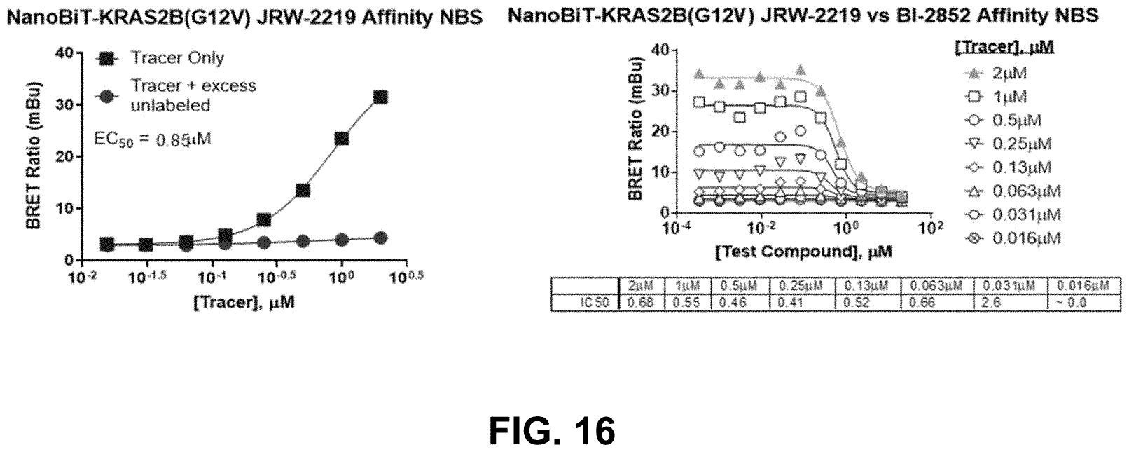

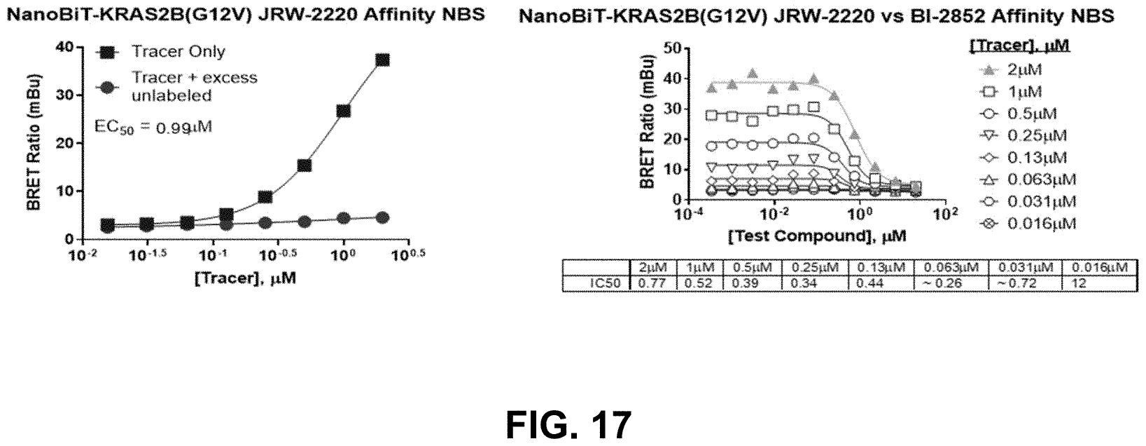

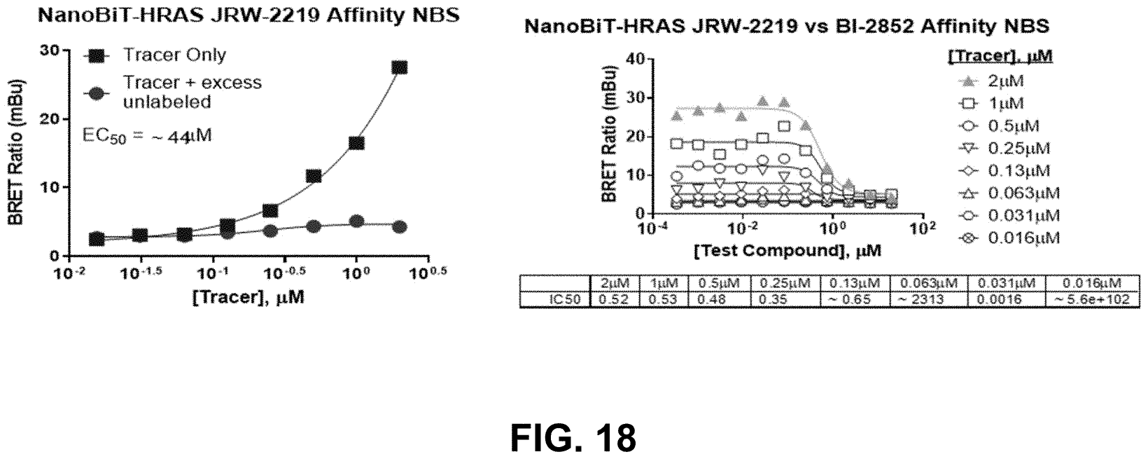

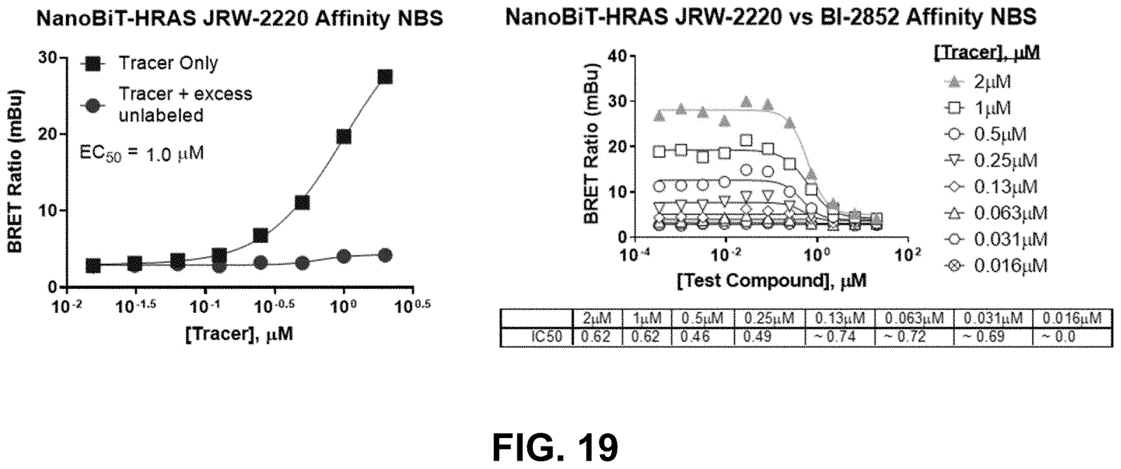

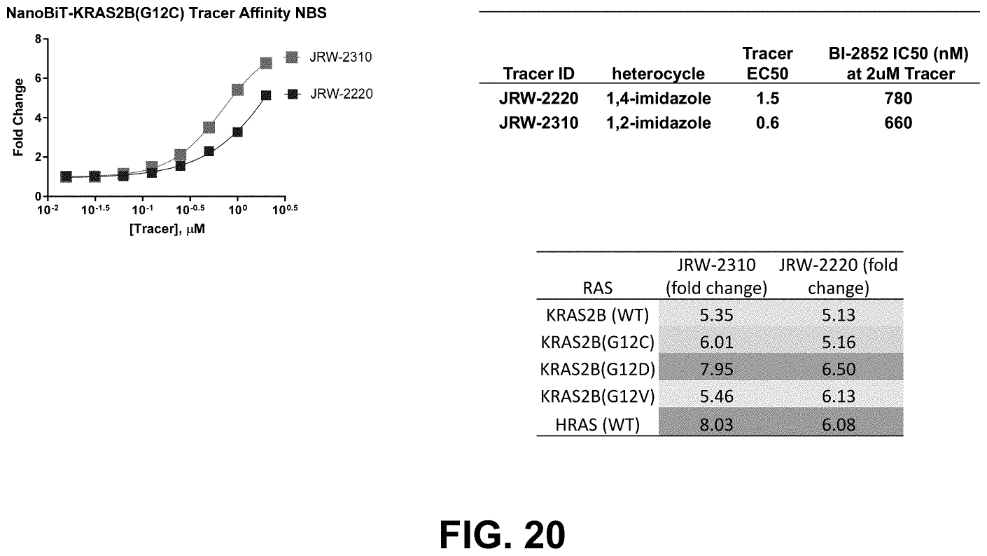

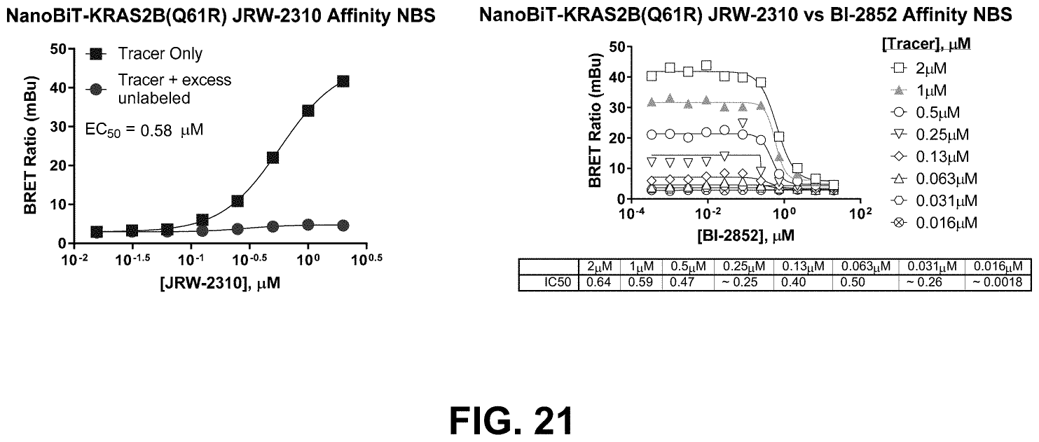

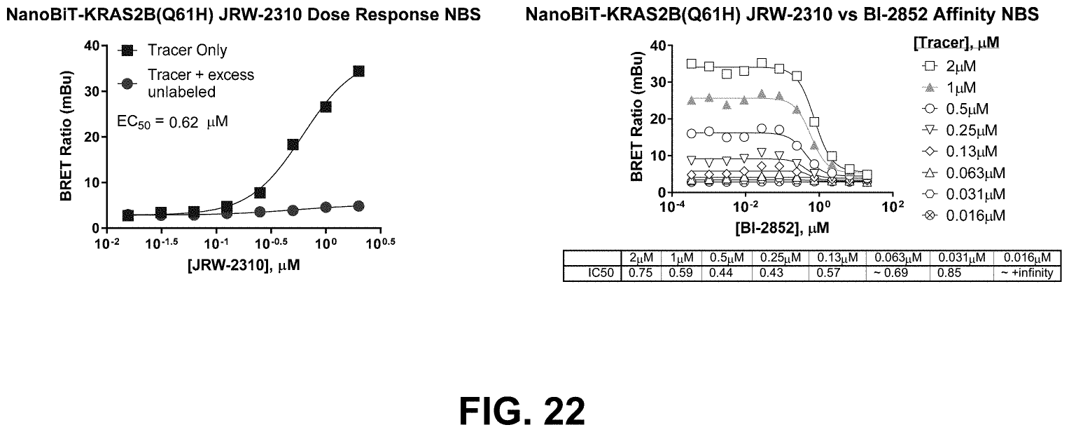

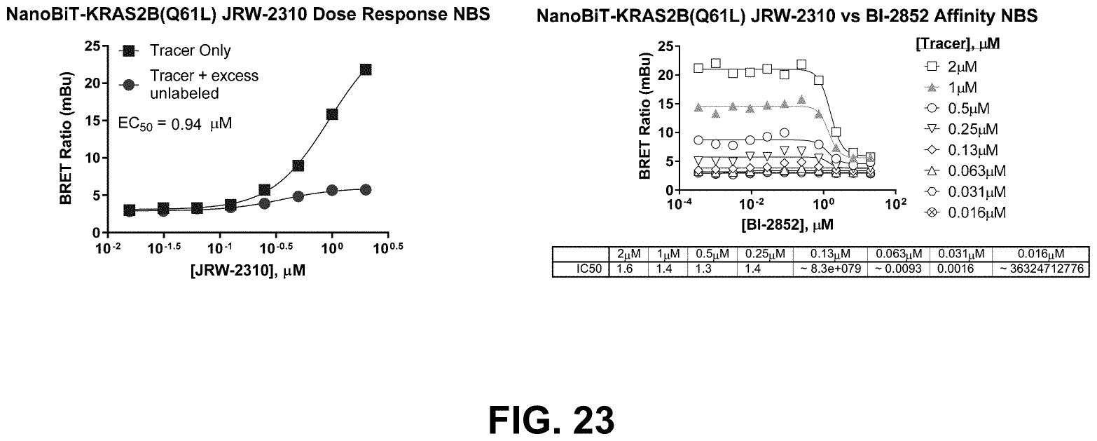

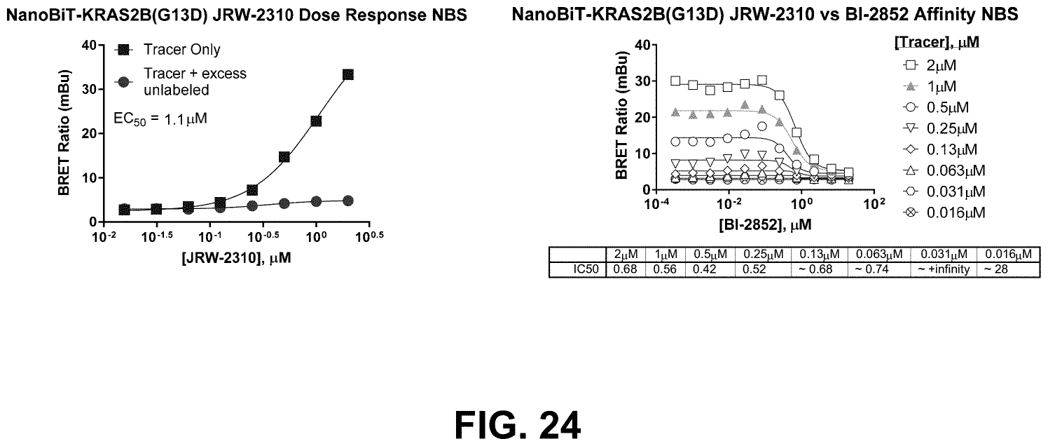

Data are shown in FIGS. 10-24. The data demonstrate that target engagement was observed in NanoBiT assays performed on cells expressing KRAS2B or a mutant thereof (KRAS2B(G12C), KRAS2B(G12D), KRAS2B(G12V), KRAS2B(Q61R), KRAS2B(Q61H), KRAS2B(Q61L), KRAS2B(G13D)), or HRAS1 as fusions with LgBiT and SmBiT using the compound JRW-2219, JRW-2220, or JRW-2310, as the RAS binding agent and the compound BI-2852 as the candidate RAS binding compound.

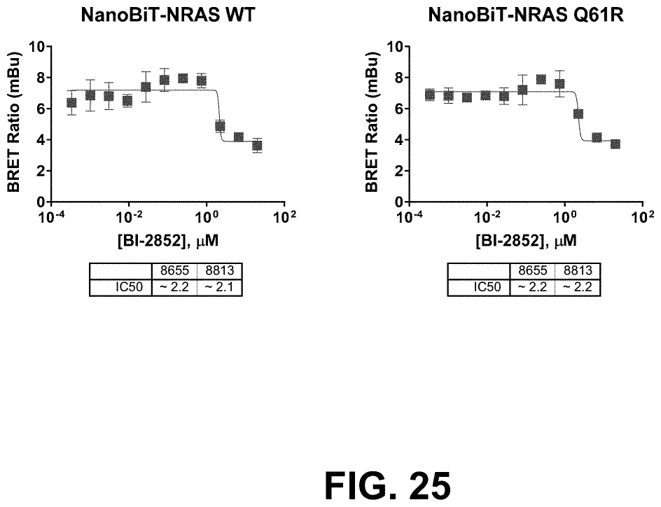

For NRAS, LgBiT-NRAS and SmBiT-NRAS plasmids were transfected into HEK293 cells at a 1:1 mass ratio, using Fugene HD at a 3:1 Lipid:DNA ratio. 24 hours post transfection, cells were harvested, seeded into corning 3600 plates at 80,000 cells/well, after which Tracer JRW-2310 was added to a final concentration of 2 μM and RAS binding compound BI-2852 was added as a dilution series. BRET was measured after a 2 hour incubation with tracer and unlabeled competitor. Data are shown in FIG. 25.

VI. Sequence
KRAS4A (nucleotide sequence isoform a)
SEQ ID NO: 1
atgactgaatataaacttgtggtagttggagctggtggcgtaggcaagagtgccttgacgatacagct
aattcagaatcattttgtggacgaatatgatccaacaatagaggattcctacaggaagcaagtagtaa
ttgatggagaaacctgtctcttggatattctcgacacagcaggtcaagaggagtacagtgcaatgagg
gaccagtacatgaggactggggagggctttctttgtgtatttgccataaataatactaaatcatttga
agatattcaccattatagagaacaaattaaaagagttaaggactctgaagatgtacctatggtcctag
taggaaataaatgtgatttgccttctagaacagtagacacaaaacaggctcaggacttagcaagaagt
tatggaattccttttattgaaacatcagcaaagacaagacagagagtggaggatgctttttatacatt
ggtgagagagatccgacaatacagattgaaaaaaatcagcaaagaagaaaagactcctggctgtgtga
aaattaaaaaatgcattataatg
KRAS4A (protein sequence of isoform a)
SEQ ID NO: 2
MTEYKLVVVGAGGVGKSALTIQLIQNHFVDEYDPTIEDSYRKQVVIDGETCLLDILDTAGQEEYSAMR
DQYMRTGEGFLCVFAINNTKSFEDIHHYREQIKRVKDSEDVPMVLVGNKCDLPSRTVDTKQAQDLARS
YGIPFIETSAKTRQRVEDAFYTLVREIRQYRLKKISKEEKTPGCVKIKKCIIM
KRAS4B (nucleotide sequence of isoform b)
SEQ ID NO: 3
atgactgaatataaacttgtggtagttggagctggtggcgtaggcaagagcaacaatagaggattcct
acaggaagcaagtagtaattgatggagaaacctgtctcttggatattctcgacacagcaggtcaagag
gagtacagtgcaatgagggaccagtacatgaggactggggagggctttctttgtgtatttgccataaa
taatactaaatcatttgaagatattcaccattatagagaacaaattaaaagagttaaggactctgaag
atgtacctatggtcctagtaggaaataaatgtgatttgccttctagaacagtagacacaaaacaggct
caggacttagcaagaagttatggaattccttttattgaaacatcagcaaagacaagacagggtgttga
tgatgccttctatacattagttcgagaaattcgaaaacataaagaaaagatgagcaaagatggtaaaa
agaagaaaaagaagtcaaagacaaagtgtgtaattatgtaa
KRAS4B (protein sequence of isoform b)
SEQ ID NO: 4
MTEYKLVVVGAGGVGKSALTIQLIQNHFVDEYDPTIEDSYRKQVVIDGETCLLDILDTAGQEEYSAMR
DQYMRTGEGFLCVFAINNTKSFEDIHHYREQIKRVKDSEDVPMVLVGNKCDLPSRTVDTKQAQDLARS
YGIPFIETSAKTRQGVDDAFYTLVREIRKHKEKMSKDGKKKKKKSKTKCVIM
KRAS4A^{G12C} (protein sequence)
SEQ ID NO: 5
MTEYKLVVVGACGVGKSALTIQLIQNHFVDEYDPTIEDSYRKQVVIDGETCLLDILDTAGQEEYSAMR
DQYMRTGEGFLCVFAINNTKSFEDIHHYREQIKRVKDSEDVPMVLVGNKCDLPSRTVDTKQAQDLARS
YGIPFIETSAKTRQRVEDAFYTLVREIRQYRLKKISKEEKTPGCVKIKKCIIM
KRAS4A^{G12D} (protein sequence)
SEQ ID NO: 6
MTEYKLVVVGADGVGKSALTIQLIQNHFVDEYDPTIEDSYRKQVVIDGETCLLDILDTAGQEEYSAMR
DQYMRTGEGFLCVFAINNTKSFEDIHHYREQIKRVKDSEDVPMVLVGNKCDLPSRTVDTKQAQDLARS
YGIPFIETSAKTRQRVEDAFYTLVREIRQYRLKKISKEEKTPGCVKIKKCIIM
KRAS4A^{G12V} (protein sequence)
SEQ ID NO: 7
MTEYKLVVVGAVGVGKSALTIQLIQNHFVDEYDPTIEDSYRKQVVIDGETCLLDILDTAGQEEYSAMR
DQYMRTGEGFLCVFAINNTKSFEDIHHYREQIKRVKDSEDVPMVLVGNKCDLPSRTVDTKQAQDLARS
YGIPFIETSAKTRQRVEDAFYTLVREIRQYRLKKISKEEKTPGCVKIKKCIIM
KRAS4B^{G12C} (protein sequence)
SEQ ID NO: 8
MTEYKLVVVGACGVGKSALTIQLIQNHFVDEYDPTIEDSYRKQVVIDGETCLLDILDTAGQEEYSAMR
DQYMRTGEGFLCVFAINNTKSFEDIHHYREQIKRVKDSEDVPMVLVGNKCDLPSRTVDTKQAQDLARS
YGIPFIETSAKTRQGVDDAFYTLVREIRKHKEKMSKDGKKKKKKSKTKCVIM
KRAS4B^{G12D} (protein sequence)
SEQ ID NO: 9
MTEYKLVVVGADGVGKSALTIQLIQNHFVDEYDPTIEDSYRKQVVIDGETCLLDILDTAGQEEYSAMR
DQYMRTGEGFLCVFAINNTKSFEDIHHYREQIKRVKDSEDVPMVLVGNKCDLPSRTVDTKQAQDLARS
YGIPFIETSAKTRQGVDDAFYTLVREIRKHKEKMSKDGKKKKKKSKTKCVIM
KRAS4B^{G12V} (protein sequence)
SEQ ID NO: 10
MTEYKLVVVGAVGVGKSALTIQLIQNHFVDEYDPTIEDSYRKQVVIDGETCLLDILDTAGQEEYSAMR
DQYMRTGEGFLCVFAINNTKSFEDIHHYREQIKRVKDSEDVPMVLVGNKCDLPSRTVDTKQAQDLARS
YGIPFIETSAKTRQGVDDAFYTLVREIRKHKEKMSKDGKKKKKKSKTKCVIM
HRAS1 (nucleotide sequence of isoform 1)
SEQ ID NO: 11
atgacggaatataagctggtggtggtgggcgccggcggtgtgggcaagagtgcgctgaccatccagct
gatccagaaccattttgtggacgaatacgaccccactatagaggattcctaccggaagcaggtggtca
ttgatggggagacgtgcctgttggacatcctggataccgccggccaggaggagtacagcgccatgcgg
gaccagtacatgcgcaccggggagggcttcctgtgtgtgtttgccatcaacaacaccaagtcttttga
ggacatccaccagtacagggagcagatcaaacgggtgaaggactcggatgacgtgcccatggtgctgg
tggggaacaagtgtgacctggctgcacgcactgtggaatctcggcaggctcaggacctcgcccgaagc
tacggcatcccctacatcgagacctcggccaagacccggcagggagtggaggatgccttctacacgtt
ggtgcgtgagatccggcagcacaagctgcggaagctgaaccctcctgatgagagtggccccggctgca
tgagctgcaagtgtgtgctctcctga
HRAS1 (protein sequence of isoform 1)
SEQ ID NO: 12
MTEYKLVVVGACGVGKSALTIQLIQNHFVDEYDPTIEDSYRKQVVIDGETCLLDILDTAGQEEYSAMR
DQYMRTGEGFLCVFAINNTKSFEDIHQYREQIKRVKDSDDVPMVLVGNKCDLAARTVESRQAQDLARS
YGIPYIETSAKTRQGVEDAFYTLVREIRQHKLRKLNPPDESGPGCMSCKCVLS
HRAS2 (nucleotide sequence of isoform 2)
SEQ ID NO: 13
atgacggaatataagctggtggtggtgggcgccggcggtgtgggcaagagtgcgctgaccatccagct
gatccagaaccattttgtggacgaatacgaccccactatagaggattcctaccggaagcaggtggtca
ttgatggggagacgtgcctgttggacatcctggataccgccggccaggaggagtacagcgccatgcgg
gaccagtacatgcgcaccggggagggcttcctgtgtgtgtttgccatcaacaacaccaagtcttttga
ggacatccaccagtacagggagcagatcaaacgggtgaaggactcggatgacgtgcccatggtgctgg
tggggaacaagtgtgacctggctgcacgcactgtggaatctcggcaggctcaggacctcgcccgaagc
tacggcatcccctacatcgagacctcggccaagacccggcagggcagccgctctggctctagctccag
ctccgggaccctctgggaccccccgggacccatgtga
HRAS2 (protein sequence of isoform 2)
SEQ ID NO: 14
MTEYKLVVVGAGGVGKSALTIQLIQNHFVDEYDPTIEDSYRKQVVIDGETCLLDILDTAGQEEYSAMR
DQYMRTGEGFLCVFAINNTKSFEDIHQYREQIKRVKDSDDVPMVLVGNKCDLAARTVESRQAQDLARS
YGIPYIETSAKTRQGSRSGSSSSSGTLWDPPGPM
HRAS1^{G12S} (protein sequence)
SEQ ID NO: 15
MTEYKLVVVGASGVGKSALTIQLIQNHFVDEYDPTIEDSYRKQVVIDGETCLLDILDTAGQEEYSAMR
DQYMRTGEGFLCVFAINNTKSFEDIHQYREQIKRVKDSDDVPMVLVGNKCDLAARTVESRQAQDLARS
YGIPYIETSAKTRQGVEDAFYTLVREIRQHKLRKLNPPDESGPGCMSCKCVLS
HRAS1^{G12V} (protein sequence)
SEQ ID NO: 16
MTEYKLVVVGAVGVGKSALTIQLIQNHFVDEYDPTIEDSYRKQVVIDGETCLLDILDTAGQEEYSAMR
DQYMRTGEGFLCVFAINNTKSFEDIHQYREQIKRVKDSDDVPMVLVGNKCDLAARTVESRQAQDLARS
YGIPYIETSAKTRQGVEDAFYTLVREIRQHKLRKLNPPDESGPGCMSCKCVLS
HRAS2^{G12S} (protein sequence)
SEQ ID NO: 17
MTEYKLVVVGASGVGKSALTIQLIQNHFVDEYDPTIEDSYRKQVVIDGETCLLDILDTAGQEEYSAMR
DQYMRTGEGFLCVFAINNTKSFEDIHQYREQIKRVKDSDDVPMVLVGNKCDLAARTVESRQAQDLARS
YGIPYIETSAKTRQGSRSGSSSSSGTLWDPPGPM
HRAS2^{G12V} (protein sequence)
SEQ ID NO: 18
MTEYKLVVVGAVGVGKSALTIQLIQNHFVDEYDPTIEDSYRKQVVIDGETCLLDILDTAGQEEYSAMR
DQYMRTGEGFLCVFAINNTKSFEDIHQYREQIKRVKDSDDVPMVLVGNKCDLAARTVESRQAQDLARS
YGIPYIETSAKTRQGSRSGSSSSSGTLWDPPGPM
NRAS (nucleotide sequence)
SEQ ID NO: 19
atgactgagtacaaactggtggtggttggagcaggtggtgttgggaaaagcgcactgacaatccagct
aatccagaaccactttgtagatgaatatgatcccaccatagaggattcttacagaaaacaagtggtta
tagatggtgaaacctgtttgttggacatactggatacagctggacaagaagagtacagtgccatgaga
gaccaatacatgaggacaggcgaaggcttcctctgtgtatttgccatcaataatagcaagtcatttgc
ggatattaacctctacagggagcagattaagcgagtaaaagactcggatgatgtacctatggtgctag
tgggaaacaagtgtgatttgccaacaaggacagttgatacaaaacaagcccacgaactggccaagagt
tacgggattccattcattgaaacctcagccaagaccagacagggtgttgaagatgctttttacacact
ggtaagagaaatacgccagtaccgaatgaaaaaactcaacagcagtgatgatgggactcagggttgta
tgggattgccatgtgtggtgatgtaa
NRAS (protein sequence)
SEQ ID NO: 20
MTEYKLVVVGAGGVGKSALTIQLIQNHFVDEYDPTIEDSYRKQVVIDGETCLLDILDTAGQEEYSAMR
DQYMRTGEGFLCVFAINNSKSFADINLYREQIKRVKDSDDVPMVLVGNKCDLPTRTVDTKQAHELAKS
YGIPFIETSAKTRQGVEDAFYTLVREIRQYRMKKLNSSDDGTQGCMGLPCVVM
NRAS^{G12D} (protein sequence)
SEQ ID NO: 21
MTEYKLVVVGADGVGKSALTIQLIQNHFVDEYDPTIEDSYRKQVVIDGETCLLDILDTAGQEEYSAMR
DQYMRTGEGFLCVFAINNSKSFADINLYREQIKRVKDSDDVPMVLVGNKCDLPTRTVDTKQAHELAKS
YGIPFIETSAKTRQGVEDAFYTLVREIRQYRMKKLNSSDDGTQGCMGLPCVVM
NRAS^{Q61R} (protein sequence)
SEQ ID NO: 22
MTEYKLVVVGAGGVGKSALTIQLIQNHFVDEYDPTIEDSYRKQVVIDGETCLLDILDTAGREEYSAMR
DQYMRTGEGFLCVFAINNSKSFADINLYREQIKRVKDSDDVPMVLVGNKCDLPTRTVDTKQAHELAKS
YGIPFIETSAKTRQGVEDAFYTLVREIRQYRMKKLNSSDDGTQGCMGLPCVVM
NanoLuc (nucleotide sequence)
SEQ ID NO: 23
atgaaacatcaccatcaccatcatgcgatcgccatggtcttcacactcgaagatttcgttggggactg
gcgacagacagccggctacaacctggaccaagtccttgaacagggaggtgtgtccagtttgtttcaga
atctcggggtgtccgtaactccgatccaaaggattgtcctgagcggtgaaaatgggctgaagatcgac
atccatgtcatcatcccgtatgaaggtctgagcggcgaccaaatgggccagatcgaaaaaatttttaa
ggtggtgtaccctgtggatgatcatcactttaaggtgatcctgcactatggcacactggtaatcgacg
gggttacgccgaacatgatcgactatttcggacggccgtatgaaggcatcgccgtgttcgacggcaaa
aagatcactgtaacagggaccctgtggaacggcaacaaaattatcgacgagcgcctgatcaaccccga
cggctccctgctgttccgagtaaccatcaacggagtgaccggctggcggctgtgcgaacgcattctgg
cggtt
NanoLuc (protein sequence)
SEQ ID NO: 24
MKHHHHHHAIAMVFTLEDFVGDWRQTAGYNLDQVLEQGGVSSLFQNLGVSVTPIQRIVLSGENGLKID
IHVIIPYEGLSGDQMGQIEKIFKVVYPVDDHHFKVILHYGTLVIDGVTPNMIDYFGRPYEGIAVFDGK
KITVTGTLWNGNKIIDERLINPDGSLLFRVTINGVTGWRLCERILAV
LgBiT (protein sequence)
SEQ ID NO: 25
MVFTLEDFVGDWEQTAAYNLDQVLEQGGVSSLLQNLAVSVTPIQRIVRSGENALKIDIHVIIPYEGLS
ADQMAQIEEVFKVVYPVDDHHFKVILPYGTLVIDGVTPNMLNYFGRPYEGIAVFDGKKITVTGTLWNG
NKIIDERLITPDGSMLFRVTINSHHHHHH
SmBiT (protein sequence)
SEQ ID NO: 26
VTGYRLFEEIL
LgTrip 3092
SEQ ID NO: 27
MVFTLDDFVGDWEQTAAYNLDQVLEQGGVSSLLQNLAVSVTPIMRIVRSGENALKIDIHVIIPYEGLS
ADQMAQIEEVFKVVYPVDDHHFKVILPYGTLVIDGVTPNKLNYFGRPYEGIAVFDGKKITVTGTLWNG
NKIIDERLITPD
LgTrip 3546
SEQ ID NO: 28
MKHHHHHHVFTLDDFVGDWEQTAAYNLDQVLEQGGVSSLLQNLAVSVTPIMRIVRSGENALKIDIHVI
IPYEGLSADQMAQIEEVFKVVYPVDDHHFKVILPYGTLVIDGVTPNKLNYFGRPYEGIAVFDGKKITT
TGTLWNGNKIIDERLITPD
LgTrip 2098
SEQ ID NO: 29
MVFTLEDFVGDWEQTAAYNLDQVLEQGGVSSLLQNLAVSVTPIQRIVRSGENALKIDIHVIIPYEGLS
ADQMAQIEEVFKVVYPVDDHHFKVILPYGTLVIDGVTPNMLNYFGRPYEGIAVFDGKKITVTGTLWNG
NKIIDERLITPD
SmTrip9
SEQ ID NO: 30
GSMLFRVTINS
5× His Tag
SEQ ID NO: 31
HHHHH
6× His Tag
SEQ ID NO: 32
HHHHHH
C-myc Tag
SEQ ID NO: 33
EQKLISEEDL
FLAG Tag
SEQ ID NO: 34
DYKDDDDK
Strep Tag
SEQ ID NO: 35
WSHPQFEK
HA Tag
SEQ ID NO: 36
YPYDVPDYA
KRAS4A^{Q61R} (protein sequence)
SEQ ID NO: 37
MTEYKLVVVGAGGVGKSALTIQLIQNHFVDEYDPTIEDSYRKQVVIDGETCLLDILDTAGREEYSAMR
DQYMRTGEGFLCVFAINNTKSFEDIHHYREQIKRVKDSEDVPMVLVGNKCDLPSRTVDTKQAQDLARS
YGIPFIETSAKTRQRVEDAFYTLVREIRQYRLKKISKEEKTPGCVKIKKCIIM
KRAS4A^{Q61H} (protein sequence)
SEQ ID NO: 38
MTEYKLVVVGAGGVGKSALTIQLIQNHFVDEYDPTIEDSYRKQVVIDGETCLLDILDTAGHEEYSAMR
DQYMRTGEGFLCVFAINNTKSFEDIHHYREQIKRVKDSEDVPMVLVGNKCDLPSRTVDTKQAQDLARS
YGIPFIETSAKTRQRVEDAFYTLVREIRQYRLKKISKEEKTPGCVKIKKCIIM
KRAS4A^{Q61L} (protein sequence)
SEQ ID NO: 39
MTEYKLVVVGAGGVGKSALTIQLIQNHFVDEYDPTIEDSYRKQVVIDGETCLLDILDTAGLEEYSAMR
DQYMRTGEGFLCVFAINNTKSFEDIHHYREQIKRVKDSEDVPMVLVGNKCDLPSRTVDTKQAQDLARS
YGIPKIETSAKTRQRVEDAFYTLVREIRQYRLKKISKEEKTPGCVKIKKCIIM
KRAS4A^{G13D} (protein sequenc)
SEQ ID NO: 40
MTEYKLVVVGAGDVGKSALTIQLIQNHFVDEYDPTIEDSYRKQVVIDGETCLLDILDTAGQEEYSAMR
DQYMRTGEGFLCVFAINNTKSFEDIHHYREQIKRVKDSEDVPMVLVGNKCDLPSRTVDTKQAQDLARS
YGIPFIETSAKTRQRVEDAFYTLVREIRQYRLKKISKEEKTPGCVKIKKCIIM
KRAS4B^{Q61R} (protein sequence)
(SEQ ID NO: 41)
MTEYKLVVVGAGGVGKSALTIQLIQNHFVDEYDPTIEDSYRKQVVIDGETCLLDILDTAGREEYSAMR
DQYMRTGEGFLCVFAINNTKSFEDIHHYREQIKRVKDSEDVPMVLVGNKCDLPSRTVDTKQAQDLARS
YGIPFIETSAKTRQGVDDAFYTLVREIRKHKEKMSKDGKKKKKKSKTKCVIM
KRAS4B^{Q61H} (protein sequence)
SEQ ID NO: 42
MTEYKLVVVGAGGVGKSALTIQLIQNHFVDEYDPTIEDSYRKQVVIDGETCLLDILDTAGHEEYSAMR
DQYMRTGEGFLCVFAINNTKSFEDIHHYREQIKRVKDSEDVPMVLVGNKCDLPSRTVDTKQAQDLARS
YGIPFIETSAKTRQGVDDAFYTLVREIRKHKEKMSKDGKKKKKKSKTKCVIM
KRAS4B^{Q61L} (protein sequence)
SEQ ID NO: 43
MTEYKLVVVGAGGVGKSALTIQLIQNHFVDEYDPTIEDSYRKQVVIDGETCLLDILDTAGLEEYSAMR
DQYMRTGEGFLCVFAINNTKSFEDIHHYREQIKRVKDSEDVPMVLVGNKCDLPSRTVDTKQAQDLARS
YGIPFIETSAKTRQGVDDAFYTLVREIRKHKEKMSKDGKKKKKKSKTKCVIM
KRAS4B^{G13D} (protein sequence)
SEQ ID NO: 44
MTEYKLVVVGAGDVGKSALTIQLIQNHFVDEYDPTIEDSYRKQVVIDGETCLLDILDTAGQEEYSAMR
DQYMRTGEGFLCVFAINNTKSFEDIHHYREQIKRVKDSEDVPMVLVGNKCDLPSRTVDTKQAQDLARS
YGIPFIETSAKTRQGVDDAFYTLVREIRKHKEKMSKDGKKKKKKSKTKCVIM

1. A method of identifying a RAS binding compound, the method comprising:
(a) providing a sample comprising a RAS protein; and
(b) contacting the sample with a RAS binding agent comprising a RAS binding moiety and a functional element, and a candidate RAS binding compound.

2. The method of claim 1, wherein the method is a method of identifying a KRAS binding compound, the method comprising:
(a) providing a sample comprising a KRAS protein; and
(b) contacting the sample with a KRAS binding agent comprising a KRAS binding moiety and a functional element, and a candidate KRAS binding compound.

3.-6. (canceled)

7. The method of claim 1, wherein the method is a method of identifying an HRAS binding compound, the method comprising:
(a) providing a sample comprising a HRAS protein; and
(b) contacting the sample with a HRAS binding agent comprising a HRAS binding moiety and a functional element, and a candidate HRAS binding compound.

8.-11. (canceled)

12. The method of claim 1, wherein the method is a method of identifying an NRAS binding compound, the method comprising:
(a) providing a sample comprising a NRAS protein; and
(b) contacting the sample with a NRAS binding agent comprising a NRAS binding moiety and a functional element, and a candidate NRAS binding compound.

13.-15. (canceled)

16. The method of claim 1, wherein step (a) comprises expressing the RAS protein within the sample.

17. The method of claim 1, wherein the RAS binding agent is a compound of formula (I): 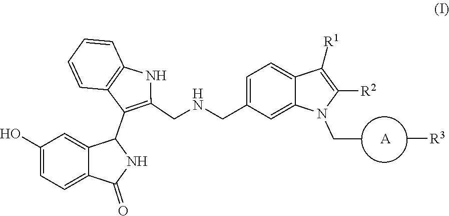
or a salt thereof, wherein:
A is a monocyclic aryl or heteroaryl;
one of R¹, R², and R³ is a group -Linker-B, wherein B is a functional element; and
the other two of R¹, R², and R³ are independently selected from hydrogen and methyl.

18. The method of claim 17, wherein A is selected from phenyl, imidazole, pyrrole, pyridyl, thiophene, and triazole.

19. The method of claim 17, wherein R¹ is a group -Linker-B, and R² and R³ are independently selected from hydrogen and methyl.

20. The method of claim 17, wherein R³ is a group -Linker-B, and R¹ and R² are independently selected from hydrogen and methyl.

21. The method of claim 17, wherein Linker has a formula: 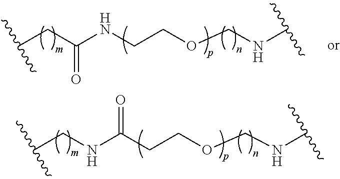
wherein m, n, and p are independently 0, 1, 2, 3, 4, 5, or 6.

22. The method of claim 1, wherein the functional element is a detectable element, an affinity element, a capture element, a solid support, or a moiety that induces protein degradation.

23. The method of claim 22, wherein the functional element is a detectable element selected from a fluorophore, chromophore, radionuclide, electron opaque molecule, MRI contrast agent, SPECT contrast agent, and mass tag.

24.-26. (canceled)

27. The method of claim 23, wherein the detectable element is a fluorophore.

28.-30. (canceled)

31. The method of claim 1, wherein the candidate RAS binding compound is a RAS inhibitor.

32. The method of claim 1, wherein the RAS binding agent binds to the RAS Switch I/II site.

33. The method of claim 1, wherein the candidate RAS binding compound binds to the RAS Switch I/II site or to the RAS Switch II site.

34. The method of claim 1, wherein the sample is selected from a cell, cell lysate, body fluid, tissue, biological sample, in vitro sample, environmental sample, cell-free sample, and purified sample (e.g., purified protein sample).

35. The method of claim 1, wherein the RAS protein is provided as a fusion with a bioluminescent reporter.

36. The method of claim 35, wherein the bioluminescent reporter is a luciferase with at least 70% sequence identity with SEQ ID NO: 24.

37. The method of claim 35, wherein the sample comprises a first RAS protein fused with a first subunit of a bioluminescent reporter, and a second RAS protein fused with a second subunit of a bioluminescent reporter, wherein the first and second subunits are complementary.

38. The method of claim 37, wherein the first subunit of the bioluminescent reporter has at least 70% sequence identity with SEQ ID NO: 25, and the second subunit of the bioluminescent reporter has at least 90% sequence identity with SEQ ID NO: 26.

39. The method of claim 35, wherein the emission spectrum of the bioluminescent reporter and the excitation spectrum of the functional element overlap.

40. The method of claim 35, further comprising contacting the sample with a substrate for the bioluminescent reporter selected from coelenterazine, a coelenterazine derivative, and furimazine.

41. (canceled)

42. A system comprising:
(a) a target RAS protein;
(b) a RAS binding agent comprising a RAS binding moiety and a functional element; and
(c) a candidate RAS binding compound.

43.-79. (canceled)

80. A RAS binding agent comprising:
(a) a RAS binding moiety; and
(b) a functional element.

81.-144. (canceled)


SEQUENCE LISTING (44 sequences)

SEQ ID NO: 1 - DNA, 567 bp, Homo sapiens
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatg

SEQ ID NO: 2 - protein, 189 aa, Homo sapiens [sequence omitted]

SEQ ID NO: 3 - DNA, 517 bp, Homo sapiens
atgactgaatataaacttgtggtagttggagctggtggcgtaggcaagagcaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 4 - protein, 188 aa, Homo sapiens [sequence omitted]

SEQ ID NO: 5 - protein, 189 aa, Homo sapiens
MTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 6 - protein, 189 aa, Homo sapiens
MTEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 7 - protein, 189 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 8 - protein, 188 aa, Homo sapiens
MTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 9 - protein, 188 aa, Homo sapiens [sequence omitted]

SEQ ID NO: 10 - protein, 188 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 11 - DNA, 570 bp, Homo sapiens
atgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 12 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 13 - DNA, 513 bp, Homo sapiens
atgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggcagccgctctggctctagctccagctccgggaccctctgggaccccccgggacccatgtga

SEQ ID NO: 14 - protein, 170 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGSRSGSSSSSGTLWDPPGPM

SEQ ID NO: 15 - protein, 189 aa, Homo sapiens
MTEYKLVVVGASGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 16 - protein, 189 aa, Homo sapiens [sequence omitted]

SEQ ID NO: 17 - protein, 170 aa, Homo sapiens
MTEYKLVVVGASGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGSRSGSSSSSGTLWDPPGPM

SEQ ID NO: 18 - protein, 170 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGSRSGSSSSSGTLWDPPGPM

SEQ ID NO: 19 - DNA, 570 bp, Homo sapiens
atgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaa

SEQ ID NO: 20 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 21 - protein, 189 aa, Homo sapiens [sequence omitted]

SEQ ID NO: 22 - protein, 189 aa, Homo sapiens
MTEYKLVVVGAGGVGKSALTIQLIQNHFVDEYDPTIEDSYRKQVVIDGETCLLDILDTAGREEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 23 - DNA, 549 bp, artificial
  synthetic
atgaaacatcaccatcaccatcatgcgatcgccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggtt

SEQ ID NO: 24 - protein, 183 aa, artificial
  synthetic
MKHHHHHHAIAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAV

SEQ ID NO: 25 - protein, 165 aa, artificial
  synthetic
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 26 - protein, 11 aa, artificial
  synthetic
VTGYRLFEEIL

SEQ ID NO: 27 - protein, 148 aa, artificial
  synthetic
MVFTLDDFVGDWEQTAAYNLDQVLEQGGVSSLLQNLAVSVTPIMRIVRSGENALKIDIHVIIPYEGLSADQMAQIEEVFKVVYPVDDHHFKVILPYGTLVIDGVTPNKLNYFGRPYEGIAVFDGKKITVTGTLWNGNKIIDERLITPD

SEQ ID NO: 28 - protein, 155 aa, artificial [sequence omitted]

SEQ ID NO: 29 - protein, 155 aa, artificial [sequence omitted]

SEQ ID NO: 30 - protein, 11 aa, artificial
  synthetic
GSMLFRVTINS

SEQ ID NO: 31 - protein, 5 aa, artificial
  synthetic
HHHHH

SEQ ID NO: 32 - protein, 6 aa, artificial
  synthetic
HHHHHH

SEQ ID NO: 33 - protein, 10 aa, artificial
  synthetic
EQKLISEEDL

SEQ ID NO: 34 - protein, 8 aa, artificial
  synthetic
DYKDDDDK

SEQ ID NO: 35 - protein, 8 aa, artificial
  synthetic
WSHPQFEK

SEQ ID NO: 36 - protein, 9 aa, artificial
  synthetic
YPYDVPDYA

SEQ ID NO: 37 - protein, 189 aa, Homo sapiens
MTEYKLVVVGAGGVGKSALTIQLIQNHFVDEYDPTIEDSYRKQVVIDGETCLLDILDTAGREEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 38 - protein, 189 aa, Homo sapiens
MTEYKLVVVGAGGVGKSALTIQLIQNHFVDEYDPTIEDSYRKQVVIDGETCLLDILDTAGHEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 39 - protein, 189 aa, Homo sapiens
MTEYKLVVVGAGGVGKSALTIQLIQNHFVDEYDPTIEDSYRKQVVIDGETCLLDILDTAGLEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 40 - protein, 189 aa, Homo sapiens
MTEYKLVVVGAGDVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 41 - protein, 188 aa, Homo sapiens
MTEYKLVVVGAGGVGKSALTIQLIQNHFVDEYDPTIEDSYRKQVVIDGETCLLDILDTAGREEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 42 - protein, 188 aa, Homo sapiens [sequence omitted]

SEQ ID NO: 43 - protein, 188 aa, Homo sapiens
MTEYKLVVVGAGGVGKSALTIQLIQNHFVDEYDPTIEDSYRKQVVIDGETCLLDILDTAGLEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 44 - protein, 188 aa, Homo sapiens [sequence omitted]